(19) United States
(12) Patent Application Publication
(10) Pub. No.: US 2025/0197416 A1
(43) Pub. Date: Jun. 19, 2025

(54) PARTHENOLIDE DERIVATIVES AND USE THEREOF

(71) Applicant: University of Rochester, Rochester, NY (US)

(72) Inventors: Rudi Fasan, Allen, TX (US); Andrew R. Bortz, Chester Springs, PA (US); John M. Bennett, Amherst, OH (US)

(73) Assignee: University of Rochester, Rochester, NY (US)

(21) Appl. No.: 18/847,368

(22) PCT Filed: Mar. 17, 2023
(86) PCT No.: PCT/US2023/064618

§ 371 Date: Sep. 16, 2024

Related U.S. Application Data
(60) Provisional application No. 63/321,198, filed on Mar. 18, 2022.

(51) Int. Cl. C07D 493/04; A61K 31/365; A61K 31/39; A61K 31/395; A61K 31/502; A61K 31/504; A61P 35/00; A61P 35/02; C07D 307/86; C07D 307/93; C07D 407/04; C07D 491/22; C07D 493/10; C07D 493/14; C07D 493/18; C07D 497/18; C07D 498/14; C07D 515/22

(52) U.S. Cl. CPC C07D 493/04; A61K 31/365; A61K 31/39; A61K 31/395; A61K 31/502; A61K 31/504; A61P 35/00; A61P 35/02; C07D 307/86; C07D 307/93; C07D 407/04; C07D 491/22; C07D 493/10; C07D 493/14; C07D 493/18; C07D 497/18; C07D 498/14; C07D 515/22

(57) ABSTRACT

An abstract heading is required. Please delete this heading section if it is not applicable to your application. For more information regarding the headings of the abstract, please see MPEP 608.01(b).

### CROSS-REFERENCE TO RELATED APPLICATIONS

This application claims priority to and the benefit of co-pending U.S. provisional patent application Ser. No. 63/321,198, entitled Complex Natural Product-Like Compounds and Use Thereof, filed Mar. 18, 2022, which is incorporated herein by reference in its entirety.

### STATEMENT REGARDING FEDERALLY SPONSORED RESEARCH OR DEVELOPMENT

This invention was made with government support under GM098628 awarded by National Institutes of Health. The government has certain rights in the invention.

### 1. TECHNICAL FIELD

The present invention relates to natural product-like compounds derived from the sesquiterpene lactone parthenolide, methods and compositions for their preparation, and methods for using these compounds in pharmaceutical compositions as anticancer and anti-inflammatory agents. The invention further relates to methods for using said natural product-like compounds for treating cancer and inflammatory diseases.

### 2. BACKGROUND OF THE INVENTION

Natural products have historically provided a major source of drugs and therapeutic agents. For example, about 50% of anticancer drugs and 60% of antibacterial drugs are natural products or semisynthetic derivatives thereof. Skeletal diversity and high stereochemical content are well known attributes that endow natural products with the ability to engage a variety of biological molecules with high affinity and specificity, leading to biological and pharmacological activity (Clemons et al. (2011). Proc. Natl. Acad. Sci. U.S.A 108, 6817-6822.; Feher et al (2003). J. Chem. Inf. Comput. Sci. 43, 218-227; Tan, D. S. (2005). Nat. Chem. Biol. 1, 74-84). Among natural products, sesquiterpene lactones (SL) are a diverse family of plant derived secondary metabolites possessing biological activity. For example, the sesquiterpene lactone parthenolide (PTL), which is found in various genera of the Asteraceae and Magnoliaceae family and is major active ingredient of the medicinal herb feverfew (Tanacetum parthenium), has found use in the traditional medicine for the treatment of pain, migraine, and rheumatoid arthritis (Heptinstall, White et al. 1985; Knight 1995). More recently, parthenolide (PTL) was found to exhibit a broad spectrum of biological properties, which include anti-inflammatory (Merfort 2011), antitumor (Ghantous, Gali-Muhtasib et al. 2010; Merfort 2011; Janecka, Wyrebska et al. 2012; Kreuger, Grootjans et al. 2012), antiviral (Hwang, Chang et al. 2006), and antileishmanic (Tiuman, Ueda-Nakamura et al. 2005) activity. The anti-inflammatory properties of parthenolide has been associated to its ability to inhibit the NF-κB transcription factor (Hehner, Heinrich et al. 1998; Garcia-Pineres, Castro et al. 2001; Garcia-Pineres, Lindenmeyer et al. 2004), which plays a prevalent role in regulating inflammatory responses (Baeuerle and Henkel 1994) as well as inhibition of other cellular mechanisms involved in inflammation, such as prostaglandin synthesis and IL-la expression (Hwang, Fischer et al. 1996) and activation of the NLRP-3 inflammasome (Juliana, Fernandes-Alnemri et al. 2010). Pharmacological inhibition of NF-κB activation, such as that induced by parthenolide, has been recognized as an important strategy for the treatment of a variety of inflammation-related pathologies, including toxic shock, asthma, and rheumatoid arthritis (Barnes and Adcock 1997; Barnes and Larin 1997). Additional studies over the past decade have also demonstrated the activity of parthenolide against different types of cancers including leukemia (Guzman, Karnischky et al. 2004; Guzman, Rossi et al. 2005; Guzman, Rossi et al. 2006), breast (Patel, Nozaki et al. 2000; Nakshatri, Rice et al. 2004; Sweeney, Mehrotra et al. 2005; Uu, Lu et al. 2008; Wyrebska, Gach et al. 2012), lung (Zhang, Qiu et al. 2009; Estabrook, Chin-Sinex et al. 2011; Shanmugam, Kusumanchi et al. 2011), prostate (Sun, St Clair et al. 2007; Kawasaki, Hurt et al. 2009; Shanmugam, Kusumanchi et al. 2010; Sun, St Clair et al. 2010), blood (Steele, Jones et al. 2006; Wang, Adachi et al. 2006; Suvannasankha, Crean et al. 2008; U, Zhang et al. 2012), colon (Zhang, Ong et al. 2004), bladder (Cheng and Xie 2011), liver (Wen, You et al. 2002; Park, Uu et al. 2005; Ralstin, Gage et al. 2006; Kim, Kim et al. 2012), skin (Won, Ong et al. 2004; Won, Ong et al. 2005; Lesiak, Koprowska et al. 2010), brain (Anderson and Bejcek 2008; Zanotto-Filho, Braganhol et al. 2011), pancreas (Kim, Uu et al. 2005; Yip-Schneider, Nakshatri et al. 2005; Yip-Schneider, Wu et al. 2008; Wang, Adachi et al. 2009; Holcomb, Yip-Schneider et al. 2012), kidney (Oka, Nishimura et al. 2007), and bone (Idris, Ubouban et al. 2009) cancer.

The anticancer and anti-inflammatory activity of parthenolide has been associated to the presence of the α-methylene-γ-lactone moiety present in its structure (Kwok, Koh et al. 2001; Hwang, Chang et al. 2006; Neelakantan, Nasim et al. 2009). Reduction of the α-methylene-γ-lactone group to give 11,13-dehydroparthenolide results in complete loss of activity (Kwok, Koh et al. 2001; Hwang, Chang et al. 2006; Neelakantan, Nasim et al. 2009). The key functional role of this structural moiety is largely related to its ability to serve as an electrophilic ‘warhead’ capable of reacting with nucleophilic sulphydryl groups in proteins, enzymes, and other cellular components (e.g., NF-κB) targeted by parthenolide in the cell (Garcia-Pineres, Castro et al. 2001; Kwok, Koh et al. 2001; Garcia-Pineres, Lindenmeyer et al. 2004; Skalska, Brookes et al. 2009). Similar to parthenolide, other sesquiterpene lactone natural products incorporating an α-methylene-γ-lactone moiety such as, for example, micheliolide, have been shown to possess anticancer activity (Ogura et al. Phytochemistry 1978, 17, 957-961; Zhang et al., J. Med. Chem. 2012, 55, 8757; An et al., PLoS One 2015, 10, e0116202).

While natural products and analogs thereof have provided a source of bioactive molecules, an alternative approach toward the discovery of new bioactive molecules is through the synthesis and screening of small molecules (e.g., <500 Da) that possess physico-chemical properties akin to those of natural products such as diverse 3-dimensional ‘shapes’ and high fraction of stereogenic centers and sp3-hybridized carbon centers (Gerry & Schreiber (2020). Curr. Opin. Chem. Biol. 56, 1-9; Wetzel et al (2011). Angew. Chem. Int. Ed. 50, 10800-10826; Wender et al. (2008). Acc. Chem. Res. 41, 40-49; Burke et al. (2004). J. Am. Chem. Soc. 126, 14095-14104; Huigens et al. (2013) Nat. Chem. 5, 195-202; Rafferty et al. (2014). Angew. Chem. Int. Ed. 53, 220-224). Indeed, it is generally recognized that bioactive molecules with natural product-like properties have higher likelihood of becoming viable drugs than less structurally complex synthetic compounds (Gerry & Schreiber (2020). Curr. Opin. Chem. Biol. 56, 1-9; Wetzel et al (2011). Angew. Chem. Int. Ed. 50, 10800-10826; Wender et al. (2008). Acc. Chem. Res. 41, 40-49; Burke et al. (2004). J. Am. Chem. Soc. 126, 14095-14104; Huigens et al. (2013) Nat. Chem. 5, 195-202; Rafferty et al. (2014). Angew. Chem. Int. Ed. 53, 220-224). In addition, complex natural product-like molecules can be useful to unveil new targets and mechanisms of action for pharmacological treatment of human diseases (Kato et al (2016). Nature 538, 344-349; Pelish et al. (2001). J. Am. Chem. Soc. 123, 6740-6741; Llabani et al (2019). Nature Chem. 11, 521-532; Liu et al (2021) Angew Chem Int Ed Engl. September 20; 60(39):21384-21395). Despite their attractive features, the synthesis of molecules with natural product-like structural complexity and diversity is far from being a trivial task and the search of bioactive compounds among them is further faced with the challenge of the virtually limitless ‘chemical space’ possible for small molecules (e.g., Reymond J-L (2015) Acc. Chem. Res. 48, 3, 722-730). There is a pressing need in the art for structurally complex and diverse bioactive molecules.

### 3. BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated, enlarged, exploded, or incomplete to facilitate an understanding of the invention.

FIG. 1 shows the synthesis of structurally and skeletally diverse natural product-like compounds via scaffold editing/rearrangement of 9(S)-hydroxy-parthenolide.

FIG. 2 shows the synthesis of additional structurally and skeletally diverse natural product-like compounds via scaffold editing/rearrangement of 9(S)-hydroxy-parthenolide.

FIG. 3 shows the synthesis of additional structurally and skeletally diverse natural product-like compounds via scaffold editing/rearrangement of 9(S)-hydroxy-parthenolide.

FIG. 4 shows the synthesis of structurally and skeletally diverse natural product-like compounds via scaffold editing/rearrangement of 1,10-epoxy-parthenolide.

FIG. 5 shows the synthesis of structurally and skeletally diverse natural product-like compounds via scaffold editing/rearrangement of 14-hydroxy-parthenolide.

FIG. 6 shows the synthesis of additional structurally and skeletally diverse natural product-like compounds via scaffold editing/rearrangement of 14-hydroxy-parthenolide.

FIG. 7 shows the synthesis of additional structurally and skeletally diverse natural product-like compounds via scaffold editing/rearrangement of 14-hydroxy-parthenolide.

FIG. 8 shows the synthesis of additional structurally and skeletally diverse natural product-like compounds via scaffold editing/rearrangement of 14-hydroxy-parthenolide.

FIG. 9A shows the anticancer activity of the compounds as measured based on reduction of cell viability upon incubation of the corresponding cancer line with the compound at 10 μM for 24 hours.

FIG. 9B shows the anticancer activity of the compounds as measured based on reduction of cell viability upon incubation of the corresponding cancer line with the compound at 20 μM for 24 hours.

FIG. 10 shows the half-maximal lethal concentration (LC₅₀) values for PTL and select compounds from the library.

FIG. 11 shows the principal component analysis (PCA) of the physiochemical properties of a representative selection of the natural product-like compounds described herein (CeDOS Library), a group of parthenolide analogs obtained via peripheral editing (LSF Library; 20 compounds), and a group of FDA-approved natural product-based drugs (NP Drugs; 42 compounds). The parent molecule parthenolide (PTL) is indicated.

FIG. 12A is a tanimoto similarity matrix of select compounds. Coefficients represent structural and skeletal similarity on a scale from 0.30 (least similar) to 1.00 (identical).

FIG. 12B is a tanimoto similarity matrix of select compounds. Coefficients represent structural and skeletal similarity on a scale from 0.30 (least similar) to 1.00 (identical).

FIG. 12C is a tanimoto similarity matrix of select compounds. Coefficients represent structural and skeletal similarity on a scale from 0.30 (least similar) to 1.00 (identical).

FIG. 13 presents a comparison of the compound library with a common commercial screening compound library. There are histograms of the ClogP values, number of stereocenters, and fraction of sp³-hybridized carbon atoms (Fₛₚ³) of the compound library, with arrows indicating the average values for the compound library, PTL, and the average value for a commercial library (ChemBridge MicroFormat Library).

### 4. DETAILED DESCRIPTION OF THE INVENTION

Methods are provided for the generation of novel natural product-like molecules obtained through the chemoenzymatic reelaboration and modification of the parthenolide scaffold. According to the methods disclosed herein, cytochrome P450-catalyzed oxyfunctionalization reactions are combined with a series of judiciously chosen molecular rearrangement and/or editing reactions resulting in the production of a structurally complex and diverse set of molecules. It is further shown that these molecules possess potent and diverse anticancer activity properties, which make suitable for application as well as further development as anticancer agents and therapeutics.

While a number of synthetic strategies have been explored for the generation of natural product-like molecules, such as using so-called “Diversity-Oriented Synthesis (DOS)”, “Complexity-to-Diversity (CtD)”, “Biology-Oriented Synthesis (BIOS)” approaches (Gerry & Schreiber (2020). Curr. Opin. Chem. Biol. 56, 1-9; Wetzel et al (2011). Angew. Chem. Int. Ed. 50, 10800-10826; Wender et al. (2008). Acc. Chem. Res. 41, 40-49; Burke et al. (2004). J. Am. Chem. Soc. 126, 14095-14104; Huigens et al. (2013) Nat. Chem. 5, 195-202; Rafferty et al. (2014). Angew. Chem. Int. Ed. 53, 220-224; Waldmann-H et al., Angew. Chem. Int. Ed. (2011) 50:10800; Park-S B et al., J. Am. Chem. Soc. (2014) 136: 14629-38), the inventors recognized that none of them utilized an enzyme-catalyzed C—H oxidation or epoxidation step as a means for generating structurally diverse new molecules from a parent scaffold. Natural or engineered cytochrome P450s can catalyze the C—H oxidation of a molecule at sites that cannot or can be hardly accessed using traditional chemical methods (e.g., Zhang et al, J Am Chem Soc. (2012); 134(45):18695-704; Kolev et al., ACS Chem Biol. (2014); 9(1):164-73; Alwaseem et al, ACS Cent Sci. (2021); 7(5):841-857). In the case of parthenolide, enzymatic methods for the generation of 9-hydroxy-parthenolide, 14-hydroxy-parthenolide, and 1,10-epoxy-parthenolide are known in the art (Kolev et al., ACS Chem Biol. (2014); 9(1):164-73) and these enzymatic transformations can be applied to obtain oxyfunctionalized derivatives of parthenolide that, while remaining not readily accessible with conventional chemical methods, can provide starting points for further modification by chemical methods.

Importantly, the inventors discovered that by combining P450-catalyzed oxyfunctionalization of parthenolide with a series of judiciously chosen chemical transformations designed to preserve the integrity of the α-methylene-g-lactone moiety, it became possible to generate new and complex molecules that possess the following desirable attributes:
(a) they exhibit high structural and stereochemical complexity akin to that found in natural products;
(b) they encompass diverse molecular skeletons and 3-dimensional shapes, which are distinct from those of the parent molecule parthenolide or simple analogs of the hydroxylated derivatives of parthenolide; and
(c) they possess an α-methylene-γ-lactone electrophilic ‘warhead’, which can be useful for covalent engagement with a biomolecular target (e.g., receptor, enzyme).

Furthermore, as a result of the skeletal rearrangement/editing transformations applied to produce them, these compounds may contain one or more alcohol functional group(s) (—OH), which can be functionalized using standard chemical procedures to produce analogs of these compounds using standard chemical methods for alcohol group interconversion. This is useful for further optimization of the pharmacological or pharmacokinetic properties of these compounds, if desired.

Furthermore, as in the case of parthenolide and micheliolide (Guzman, Rossi et al. 2006; Hwang, Chang et al. 2006; Nasim and Crooks 2008; Han, Barrios et al. 2009; Neelakantan, Nasim et al. 2009; Woods, Mo et al. 2011; Zhang et al, J. Med. Chem. 2012, 55, 8757), the α-methylene-g-lactone moiety in the compounds disclosed herein can be further reacted with an amine-based nucleophile (e.g., dimethylamine, diethylamine, dipropylamine, pyrrolidine, and the like) to produce an amine-adduct analog, or prodrug, which possesses improved water-solubility while retaining most of the biological activity of the parent molecule.

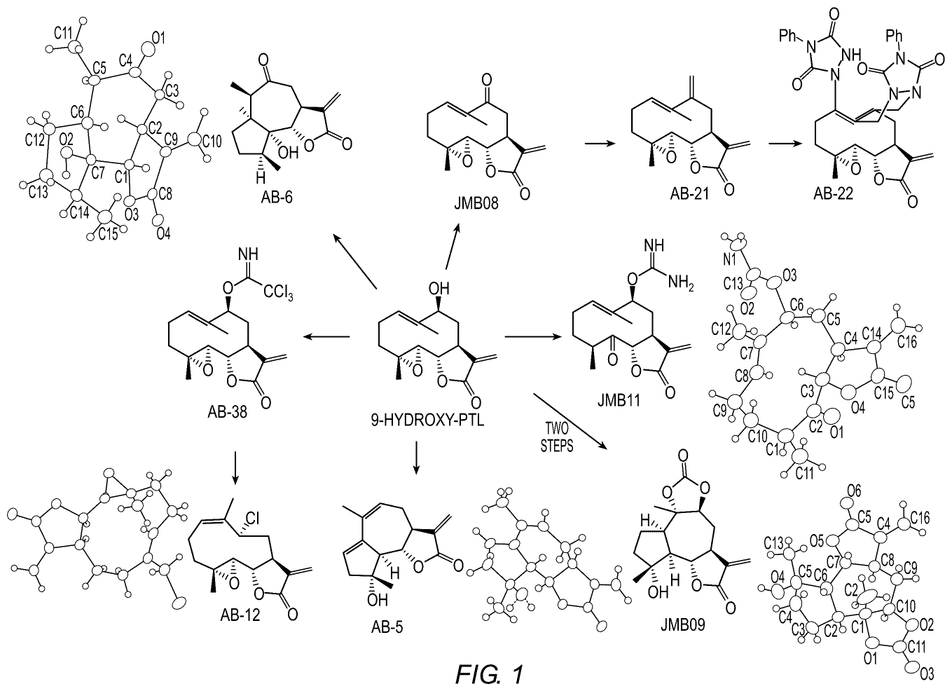

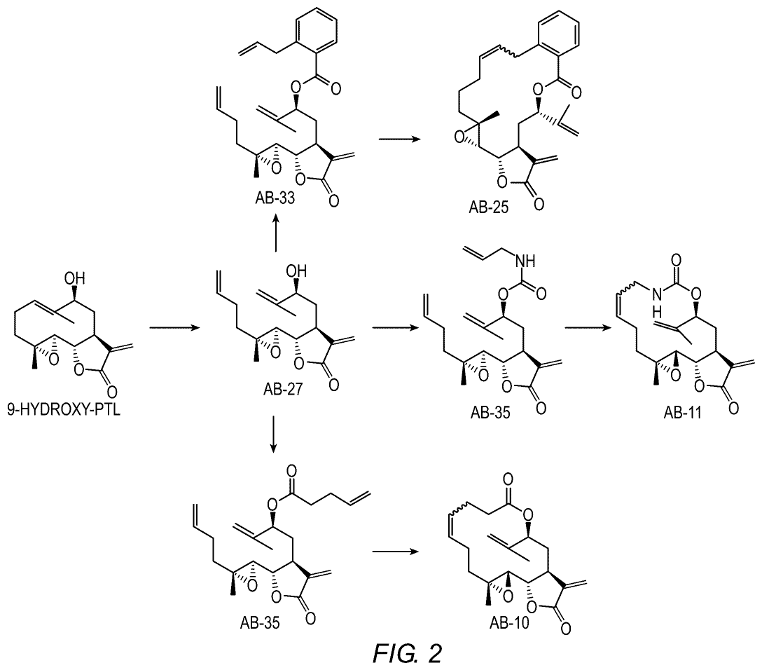

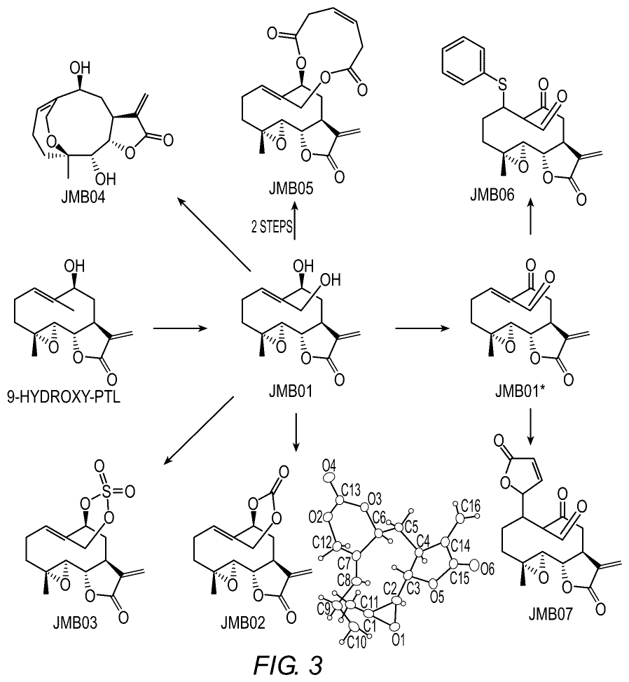

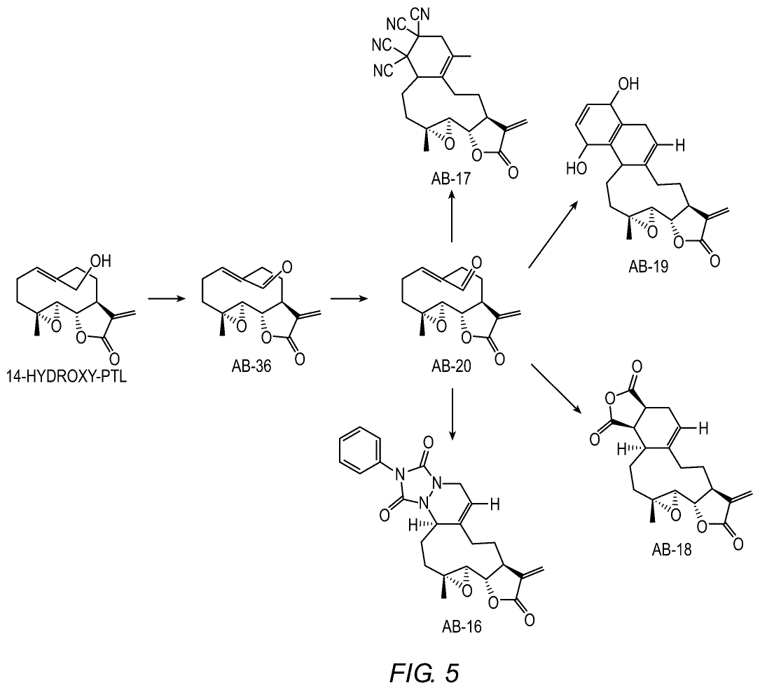

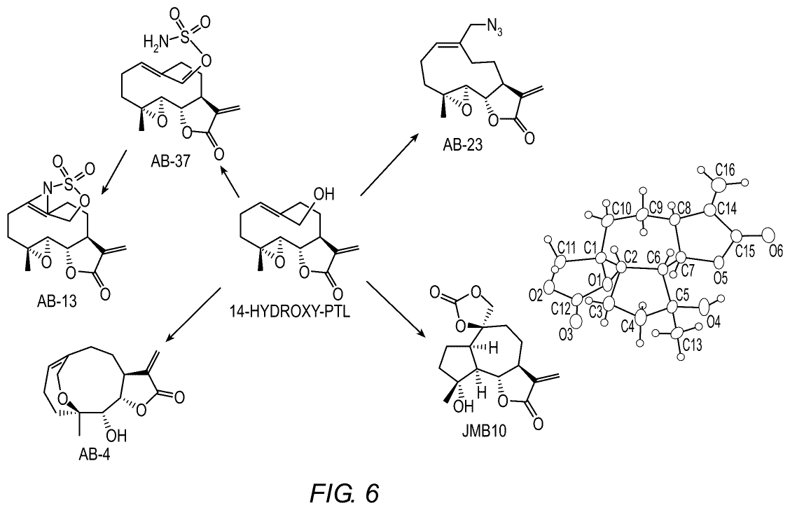

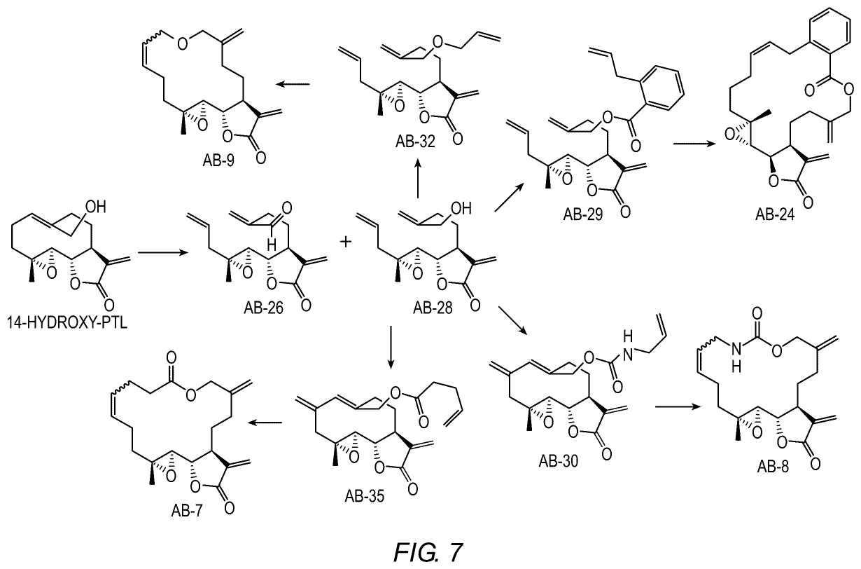

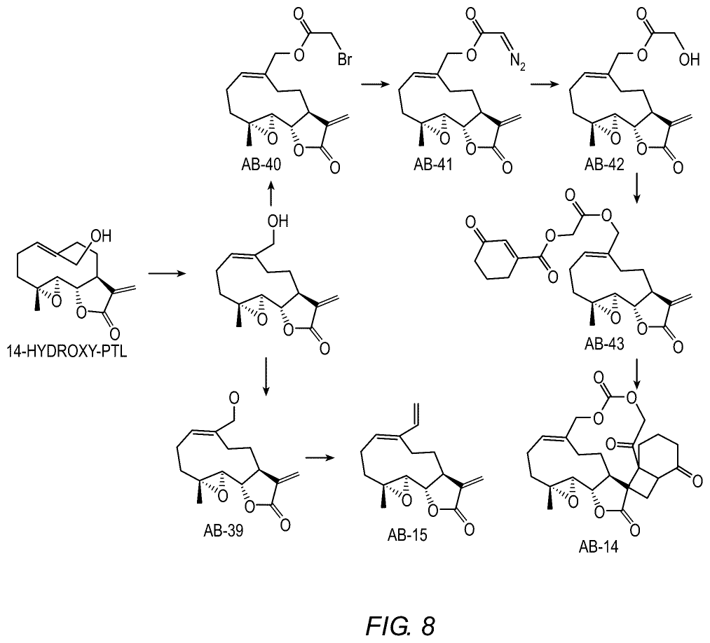

Exemplary synthetic routes for the preparation of the compounds disclosed herein are shown in FIGS. 1-8.

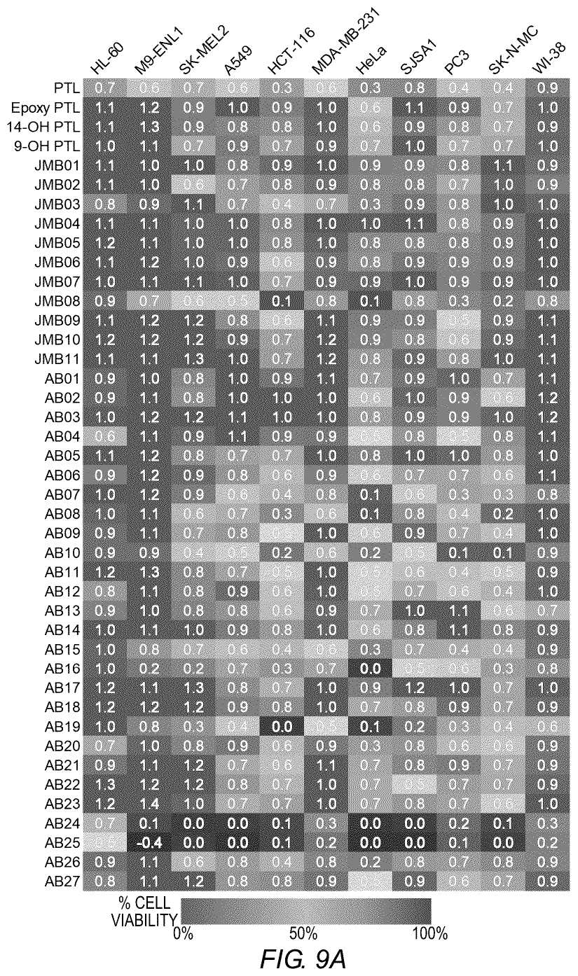

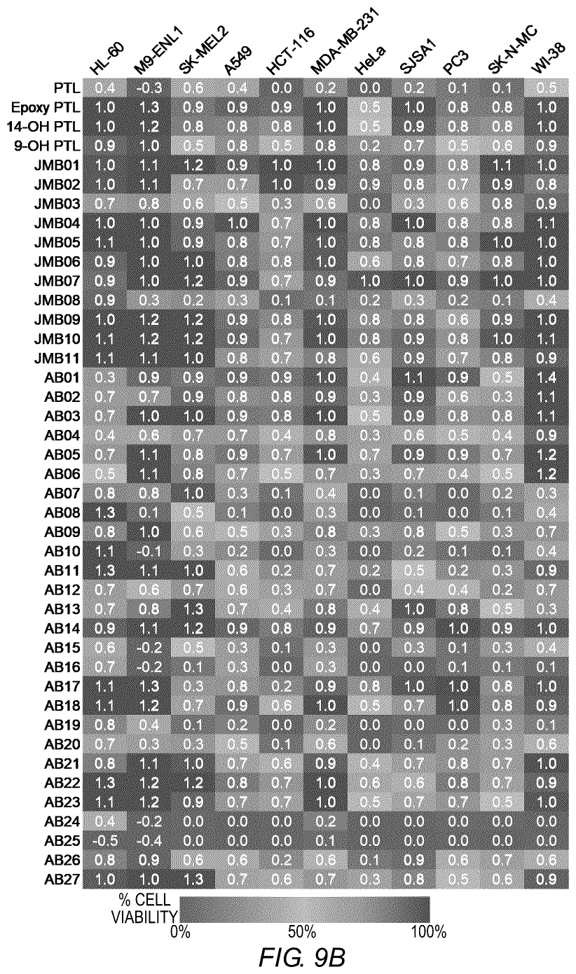

The inventors further discovered that these compounds possess anticancer activity (FIG. 9). Furthermore, these compounds may possess other biological activities, including antimicrobial, antibiotic, antifungal, and anti-inflammatory activity.

For clarity of disclosure, and not by way of limitation, the detailed description of the invention is divided into the subsections set forth below.

### 4.1 Definitions

The term “functional group” as used herein refers to a contiguous group of atoms that, together, may undergo a chemical reaction under certain reaction conditions. Examples of functional groups are, among many others, —OH, —NH₂, —SH, —(C═O)—, —Na, —C≡CH.

The term “aliphatic” or “aliphatic group” as used herein means a straight or branched C₁₋₁₅ hydrocarbon chain that is completely saturated or that contains one or more units of unsaturation, or a monocyclic C₃₋₈ hydrocarbon, or bicyclic C₈₋₁₂ hydrocarbon that is completely saturated or that contains one or more units of unsaturation, but which is not aromatic (also referred to herein as “cycloalkyl”). For example, suitable aliphatic groups include, but are not limited to, linear or branched alkyl, alkenyl, alkynyl groups or hybrids thereof such as (cycloalkyl)alkyl, (cycloalkenyl)alkyl, or (cycloalkynyl)alkyl. The alkyl, alkenyl, or alkynyl group may be linear, branched, or cyclic and may contain up to 15, preferably up to 8, and most preferably up to 5 carbon atoms. Preferred alkyl groups include methyl, ethyl, propyl, cyclopropyl, butyl, cyclobutyl, pentyl, and cyclopentyl groups. Preferred alkenyl groups include propenyl, butenyl, and pentenyl groups. Preferred alkynyl groups include propynyl, butynyl, and pentynyl groups.

The term “aryl” and “aryl group” as used herein refers to an aromatic substituent containing a single aromatic or multiple aromatic rings that are fused together, directly linked, or indirectly linked (such as linked through a methylene or an ethylene moiety). An aryl group may contain from 5 to 24 carbon atoms, preferably 5 to 18 carbon atoms, and most preferably 5 to 14 carbon atoms.

The terms “heteroatom” means nitrogen, oxygen, or sulfur, and includes any oxidized forms of nitrogen and sulfur, and the quaternized form of any basic nitrogen. Heteroatom further include Se, Si, and P.

The term “heteroaryl” as used herein refer to an aryl group in which at least one carbon atom is replaced with a heteroatom. Preferably, a heteroaryl group is a 5- to 18-membered, particularly a 5- to 14-membered, and especially a 5- to 10-membered aromatic ring system containing at least one heteroatom selected from the group consisting of oxygen, sulfur, and nitrogen atoms. Preferred heteroaryl groups include pyridyl, pyrrolyl, furyl, thienyl, indolyl, isoindolyl, indolizinyl, imidazolyl, pyridonyl, pyrimidyl, pyrazinyl, oxazolyl, thiazolyl, purinyl, quinolinyl, isoquinolinyl, benzofuranyl, and benzoxazolyl groups.

A heterocyclic group may be any monocyclic or polycyclic ring system which contains at least one heteroatom and may be unsaturated or partially or fully saturated. The term “heterocyclic” thus includes heteroaryl groups as defined above as well as non-aromatic heterocyclic groups. Preferably, a heterocyclic group is a 3- to 18-membered, particularly a 3- to 14-membered, and especially a 3- to 10-membered, ring system containing at least one heteroatom selected from the group consisting of oxygen, sulfur, and nitrogen atoms. Preferred heterocyclic groups include the specific heteroaryl groups listed above as well as pyranyl, piperidinyl, pyrrolidinyl, dioaxanyl, piperazinyl, morpholinyl, thiomorpholinyl, morpholinosulfonyl, tetrahydroisoquinolinyl, and tetrahydrofuranyl groups.

A halogen atom may be a fluorine, chlorine, bromine, or iodine atom.

By “optionally substituted”, it is intended that in the any of the chemical groups listed above (e.g., alkyl, alkenyl, alkynyl, heteroalkyl, heteroalkenyl, heteroalkynyl, aryl, heteroaryl, heterocyclic, triazolyl groups), one or more hydrogen atoms are optionally replaced with an atom or chemical group other than hydrogen. Specific examples of such substituents include, without limitation, halogen atoms, hydroxyl (—OH), sulfhydryl (—SH), substituted sulfhydryl, carbonyl (—CO—), carboxy (—COOH), amino (—NH₂), nitro (—NO₂), sulfo (—SO₂—OH), cyano (—C═N), thiocyanato (—S—CEN), phosphono (—P(O)OH₂), alkyl, alkenyl, alkynyl, heteroalkyl, heteroalkenyl, heteroalkynyl, aryl, heteroaryl, heterocyclic, alkylthiol, alkyloxy, alkylamino, aryithiol, aryloxy, or arylamino groups. Where “optionally substituted” modifies a series of groups separated by commas (e.g., “optionally substituted A, B, or C”; or “A, B, or C optionally substituted with”), it is intended that each of the groups (e.g., A, B, or C) is optionally substituted.

The term “α-methylene-γ-lactone” as used herein refers to a 3-methylene-dihydrofuran-2(3H)-one group.

The term “contact” as used herein with reference to interactions of chemical units indicates that the chemical units are at a distance that allows short range non-covalent interactions (such as Van der Waals forces, hydrogen bonding, hydrophobic interactions, electrostatic interactions, dipole-dipole interactions) to dominate the interaction of the chemical units. For example, when a protein is ‘contacted’ with a chemical species, the protein is allowed to interact with the chemical species so that a reaction between the protein and the chemical species can occur.

The term “polypeptide”, “protein”, and “enzyme” as used herein refers to any chain of two or more amino acids bonded in sequence, regardless of length or post-translational modification. According to their common use in the art, the term “protein” refers to any polypeptide consisting of more than 50 amino acid residues. These definitions are however not intended to be limiting.

In general, the term “mutant” or “variant” as used herein with reference to a molecule such as polynucleotide or polypeptide, indicates that such molecule has been mutated from the molecule as it exists in nature. In particular, the term “mutate” and “mutation” as used herein indicates any modification of a nucleic acid and/or polypeptide which results in an altered nucleic acid or polypeptide. Mutations include any process or mechanism resulting in a mutant protein, enzyme, polynucleotide, or gene. A mutation can occur in a polynucleotide or gene sequence, by point mutations, deletions, or insertions of single or multiple nucleotide residues. A mutation in a polynucleotide includes mutations arising within a protein-encoding region of a gene as well as mutations in regions outside of a protein-encoding sequence, such as, but not limited to, regulatory or promoter sequences. A mutation in a coding polynucleotide such as a gene can be “silent”, i.e., not reflected in an amino acid alteration upon expression, leading to a “sequence-conservative” variant of the gene. A mutation in a polypeptide includes but is not limited to mutation in the polypeptide sequence and mutation resulting in a modified amino acid. Non-limiting examples of a modified amino acid include a glycosylated amino acid, a sulfated amino acid, a prenylated (e.g., farnesylated, geranylgeranylated) amino acid, an acetylated amino acid, an acylated amino acid, a PEGylated amino acid, a biotinylated amino acid, a carboxylated amino acid, a phosphorylated amino acid, and the like.

The term “engineer” or “engineered” refers to any manipulation of a molecule that result in a detectable change in the molecule, wherein the manipulation includes but is not limited to inserting a polynucleotide and/or polypeptide heterologous to the cell and mutating a polynucleotide and/or polypeptide native to the cell.

The term “polynucleotide molecule” as used herein refers to any chain of two or more nucleotides bonded in sequence. For example, a nucleic acid molecule can be a DNA or an RNA.

The terms “vector” and “vector construct” as used herein refer to a vehicle by which a DNA or RNA sequence (e.g., a foreign gene) can be introduced into a host cell, so as to transform the host and promote expression (e.g., transcription and translation) of the introduced sequence. A common type of vector is a “plasmid”, which generally is a self-contained molecule of double-stranded DNA that can be readily accept additional (foreign) DNA and which can be readily introduced into a suitable host cell. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. The terms “express” and “expression” refer to allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be the to be “expressed” by the cell. A polynucleotide or polypeptide is expressed recombinantly, for example, when it is expressed or produced in a foreign host cell under the control of a foreign or native promoter, or in a native host cell under the control of a foreign promoter.

### 4.3 Complex Natural Product-Like Compounds

A compound of general formula A1 through A45, or a salt thereof, is provided:

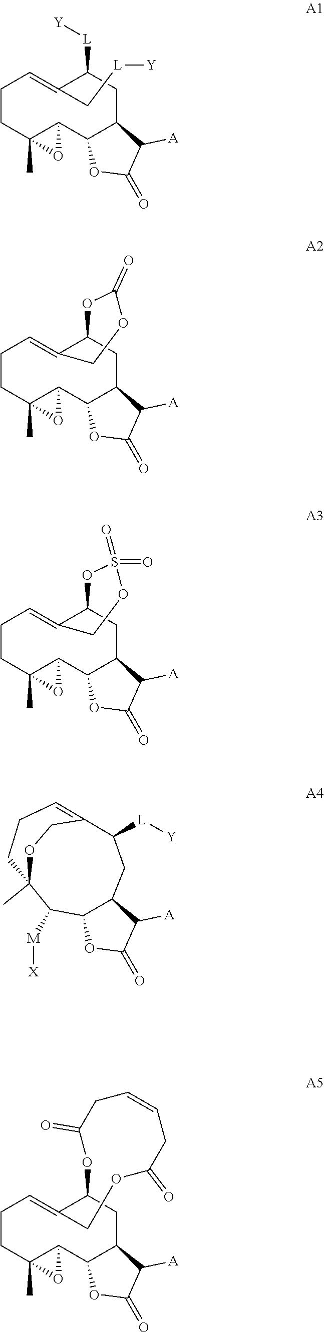

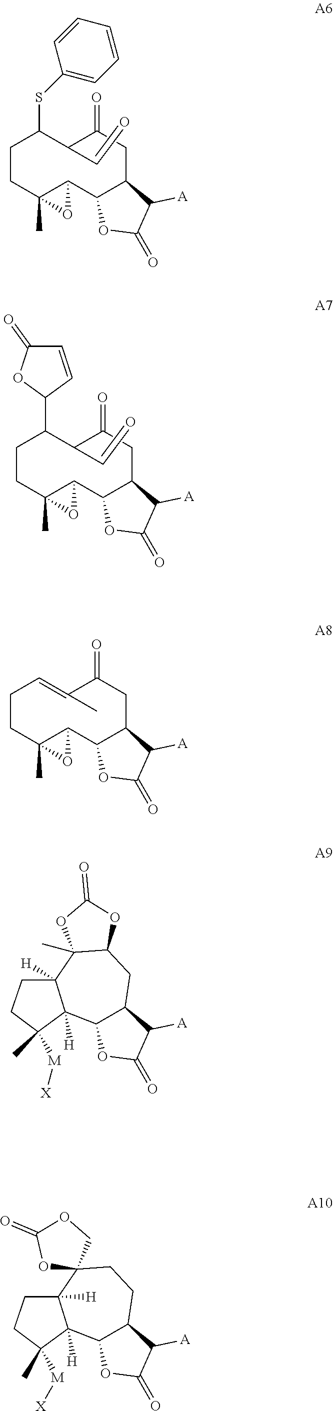

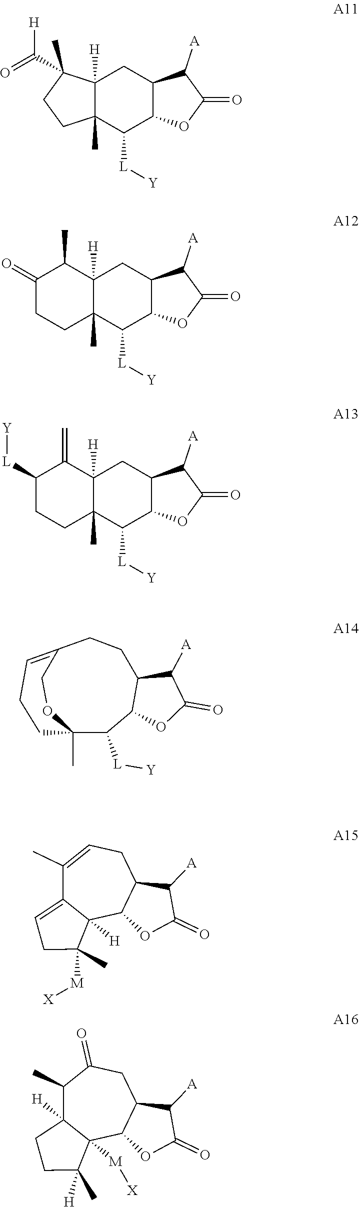

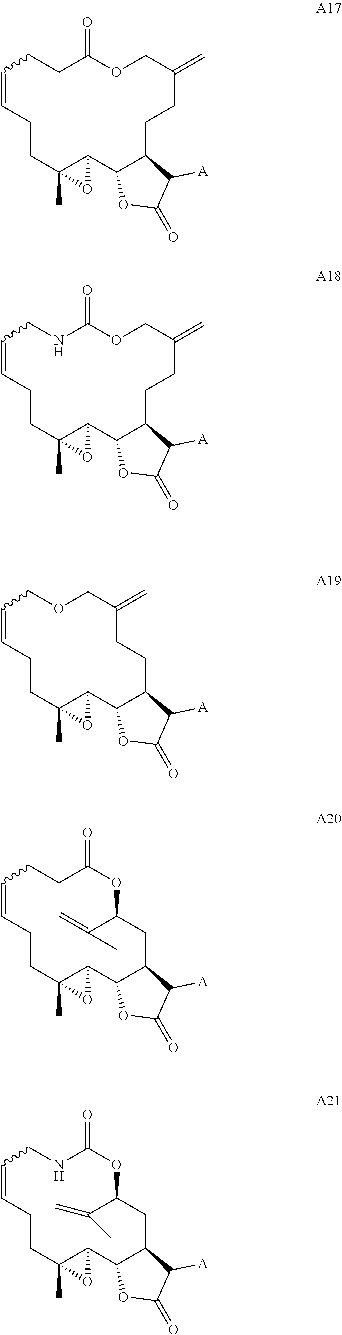

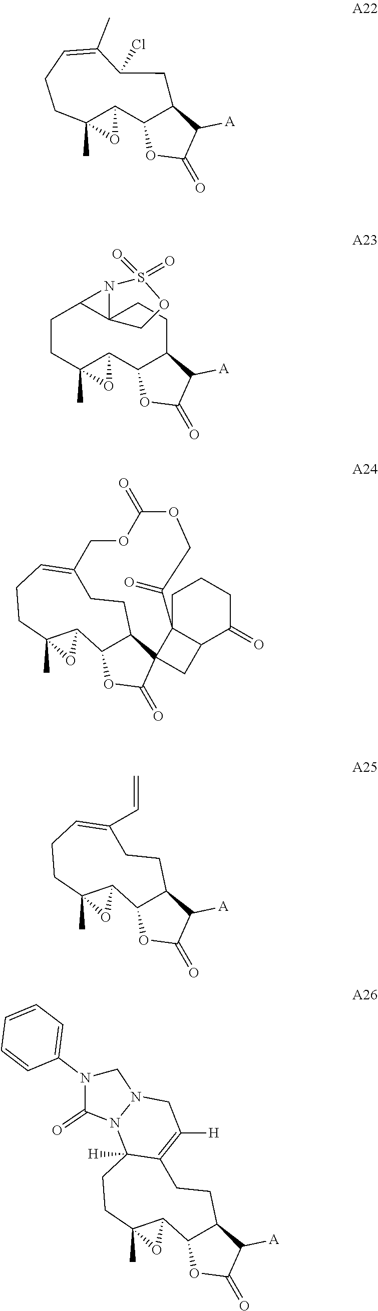

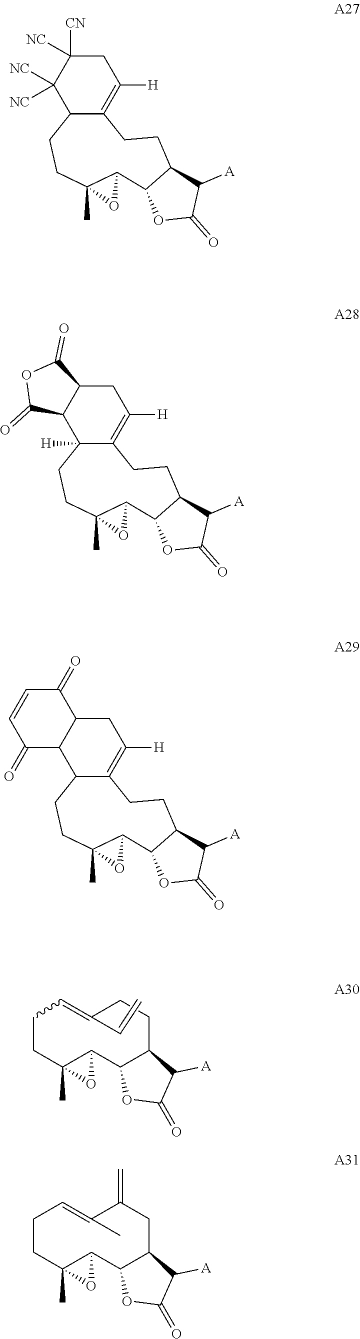

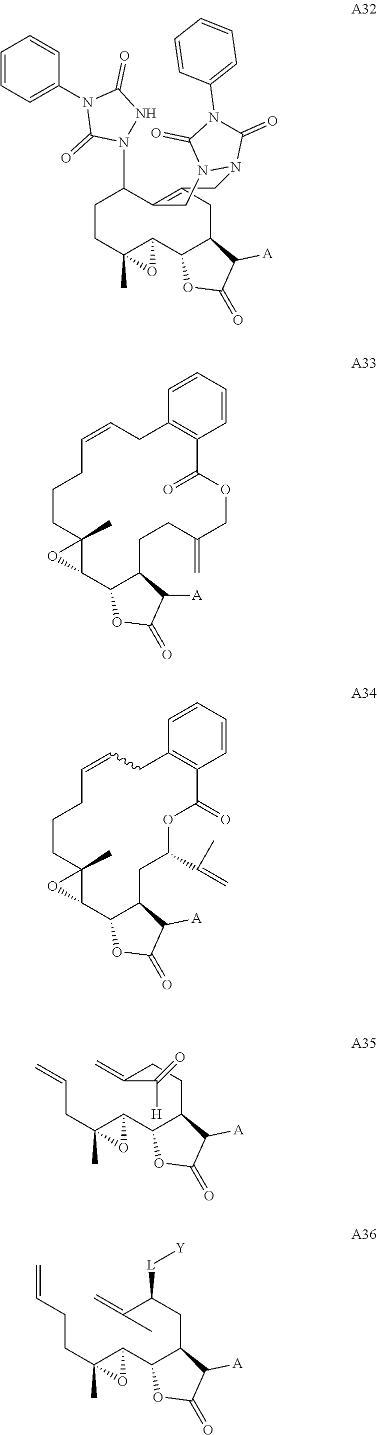

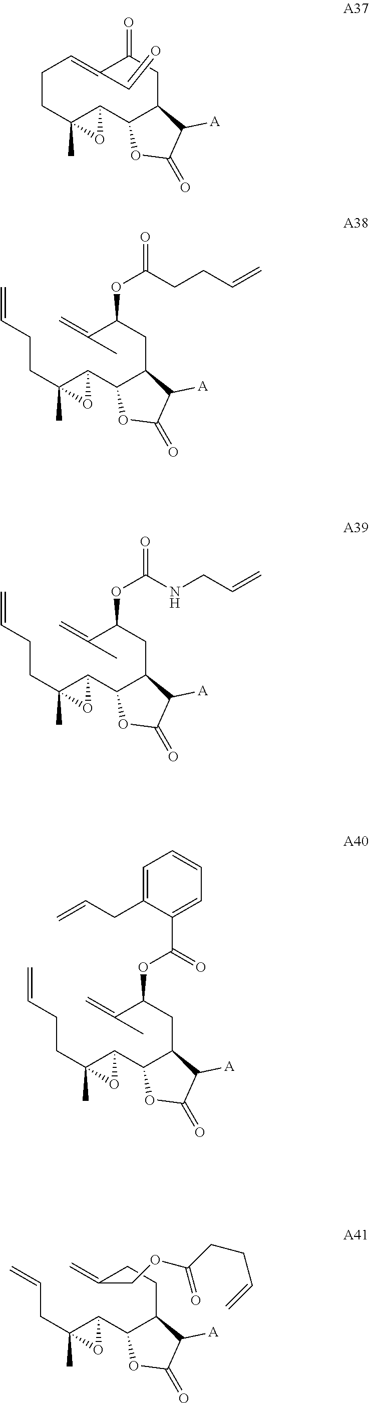

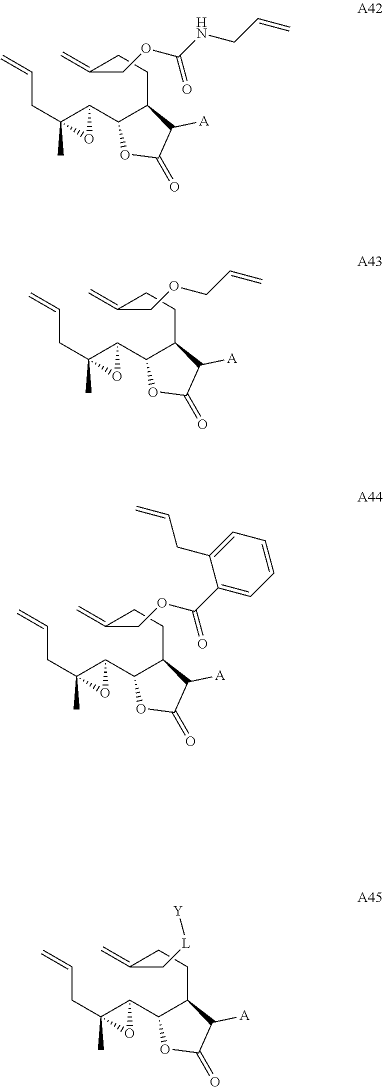

wherein
A is ═CH₂ or —CH₂R* wherein R* is an amino acid residue bonded to the A methylene via a nitrogen or sulfur atom; or R* is —NR¹R², —NR¹C(O)R², —NR¹CO₂R², or —SR¹, wherein R¹ and R² are independently selected from the group consisting of H and an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group; or R* is —NR¹R², wherein R₁ and R₂ optionally together with the nitrogen atom form an optionally substituted 5-12 membered ring, the ring optionally comprising at least one heteroatom or group selected from the group consisting of —CO—, —SO—, —SO₂—, and —PO—;
L is —O—, —NH—, —NHC(O)—, —OC(O)—, —OC(O)NH—, —S—, —SO—, —SO₂—, —PO—, —OCH₂—, or a chemical bond connecting the carbon atom to Y; and Y represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group, with the proviso that when -L-Y is hydrogen, -M-X is not hydroxyl, nor —C(O)CH₂CH₃ and A is not —CH₂—N(CH₃)₂, wherein -L-Y together with the carbon atom at position C2 optionally forms carbonyl; or
Y is absent and L represents a halogen atom, an azido group (—N₃), an optionally substituted triazole group, or a group —NR³R⁴, where R³ represents a hydrogen atom or an optionally substituted alkyl, alkenyl, or alkynyl group; R⁴ represents an optionally substituted alkyl, alkenyl, alkynyl, aryl, or heteroaryl group; or where R³ and R⁴ are connected together to form an optionally substituted heterocyclic group;
M is —O— or —OC(O)— and X represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group.

In some embodiments, in the compound of general formula A1 through A45, L and M are independently —O— or —OC(O)—; Y and X are independently hydrogen, —(C6-C10) aryl, -(5-14 membered) heteroaryl, —(C1-C6) alkyl, —CH2-(C6-C10) aryl, or —NH—(C6-C10) aryl; A is CH2R*, where R* is selected from the group consisting of methylamino (—NH(CH3)), dimethylamino (—N(CH3)₂), methylethylamino (—N(CH3)(CH2CH3)), methylpropylamino (—N(CH3)(CH2CH2CH3)), methylisopropylamino (—(CH3)(CH2(CH3)₂), (—N(CH3)(CH2CH2OH), pyrrolidine, piperidine, 4-methylpiperidine, 1-phenylmethanamine (—NCH2Ph), and 2-phenylethanamine (—NCH2CHPh). In other embodiments, A is ═CH₂. In other embodiments, A is —CH₂R* wherein R* is an amino acid residue bonded to the A methylene via a nitrogen or sulfur atom. In other embodiments, R* is —NR¹R², —NR¹C(O)R², —NR¹CO₂R², or —SR¹, wherein R¹ and R² are independently selected from the group consisting of H and an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group. In some embodiments, R* is —NR¹R², wherein R₁ and R₂ optionally together with the nitrogen atom form an optionally substituted 5-12 membered ring. In some embodiments, the ring may comprise at least one heteroatom or group selected from the group consisting of —CO—, —SO—, —SO₂—, and —PO—.

In some embodiments, L is —O—, —NH—, —NHC(O)—, —OC(O)—, —OC(O)NH—, —S—, —SO—, —SO₂—, —PO—, —OCH₂—, or a chemical bond connecting the carbon atom to Y; and Y represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group, with the proviso that when -L-Y is hydrogen, -M-X is not hydroxyl, nor —C(O)CH₂CH₃ and A is not —CH₂—N(CH₃)₂, wherein -L-Y together with the carbon atom at position C2 optionally forms carbonyl. In other embodiments, L represents a halogen atom, an azido group (—N₃), an optionally substituted triazole group, or a group —NR³R⁴, where R³ represents a hydrogen atom or an optionally substituted alkyl, alkenyl, or alkynyl group; R⁴ represents an optionally substituted alkyl, alkenyl, alkynyl, aryl, or heteroaryl group; or where R³ and R⁴ are connected together to form an optionally substituted heterocyclic group. In some embodiments, M is —O— or —OC(O)— and X represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group.

In some embodiments, the present invention provides compounds of formula (A), wherein L is —OC(O)—; Y is hydrogen, —(C₆-C₁₀) aryl, -(5-14 membered) heteroaryl, —(C₁-C₆) alkyl, —CH₂—(C₆-C₁₀) aryl, or —NH—(C₆-C₁₀) aryl, each of which is optionally substituted with 1 to 3 substituents selected from the group consisting of —CF₃, —N(CH₃)₂, —CH₃, halogen, phenyl and phenyl substituted with 1 to 2 substituents selected from —CF₃ and halogen; A is ═CH₂, or CH₂R*, where R* is selected from the group consisting of methylamino (—NH(CH₃)), dimethylamino (—N(CH₃)₂), methylethylamino (—N(CH₃)(CH₂CH₃)), methylpropylamino (—N(CH₃)(CH₂CH₂CH₃)), methylisopropylamino (—(CH₃)(CH₂(CH₃)₂), (—N(CH₃)(CH₂CH₂OH), pyrrolidine, piperidine, 4-methylpiperidine, 1-phenylmethanamine (—NCH₂Ph), and 2-phenylethanamine (—NCH₂CHPh); M is —O—, or —OC(O)—; X is hydrogen, —(C₆-C₁₀) aryl, -(5-14 membered) heteroaryl, (C₁-C₆) alkyl, -—N(CH₃)₂—(C₆-C₁₀) aryl, each of which is optionally substituted with 1 to 3 substituents selected from the group consisting of —CF₃, —N(CH₃)₂, —CH₃, halogen, phenyl and phenyl substituted with 1 to 2 substituents selected from —CF₃ and halogen. Preferably, L is —OC(O)—; Y is —(C₆-C₁₀) aryl, -(5-14 membered) heteroaryl, or —NH—(C₆-C₁₀) aryl, each of which is optionally substituted with 1 to 3 substituents selected from the group consisting of —CF₃, halogen, phenyl, and phenyl substituted with 1 to 2 substituents selected from —CF₃ and halogen; A is ═CH₂; M is —O—, or —OC(O)—; X is hydrogen, —(C₆-C₁₀) aryl, -(5-14 membered) heteroaryl, each of which is optionally substituted with 1 to 3 substituents selected from the group consisting of —CF₃, —N(CH₃)₂, —CH₃, halogen, phenyl and phenyl substituted with 1 to 2 substituents selected from —CF₃ and halogen.

In some embodiments, the present invention provides compounds of formula (A), wherein L is —OC(O)—. In some embodiments, Y is selected from the group consisting of hydrogen, —(C₆-C₁₀) aryl, -(5-14 membered) heteroaryl, —(C₁-C₆) alkyl, —CH₂—(C₆-C₁₀) aryl, or —NH—(C₆-C₁₀) aryl. Each of these compounds may be optionally substituted with 1 to 3 substituents selected from the group consisting of —CF₃, —N(CH₃)₂, —CH₃, halogen, phenyl and phenyl substituted with 1 to 2 substituents selected from —CF₃ and halogen. In some embodiments, A is ═CH₂, or CH₂R*, where R* is selected from the group consisting of methylamino (—NH(CH₃)), dimethylamino (—N(CH3)2), methylethylamino (—N(CH₃)(CH₂CH₃)), methylpropylamino (—N(CH₃)(CH₂CH₂CH₃)), methylisopropylamino (—(CH₃)(CH₂(CH₃)₂), (—N(CH₃)(CH₂CH₂OH), pyrrolidine, piperidine, 4-methylpiperidine, 1-phenylmethanamine (—NCH₂Ph), and 2-phenylethanamine (—NCH₂CHPh). In some embodiments, M is —O—, or —OC(O). In some embodiments, X is selected from the group consisting of hydrogen, —(C₆-C₁₀) aryl, -(5-14 membered) heteroaryl, (C₁-C₆) alkyl, —N(CH₃)₂-(C₆-C₁₀) aryl. Each of these compounds may be optionally substituted with 1 to 3 substituents selected from the group consisting of —CF₃, —N(CH₃)₂, —CH₃, halogen, phenyl and phenyl substituted with 1 to 2 substituents selected from —CF₃ and halogen. Preferably, L is —OC(O)—; Y is —(C₆-C₁₀) aryl, -(5-14 membered) heteroaryl, or —NH—(C₆-C₁₀) aryl. In some embodiments, each of these compounds may be optionally substituted with 1 to 3 substituents selected from the group consisting of —CF₃, halogen, phenyl, and phenyl substituted with 1 to 2 substituents selected from —CF₃ and halogen. In some embodiments, the present invention provides compounds of formula (A), wherein L is —O—; Y is —C(O)—(C₆-C₁₀) aryl, —C(O)— -(5-14 membered) heteroaryl, hydrogen, —CH₂—(C₆-C₁₀) aryl, —C(O)—NH—(C₆-C₁₀) aryl, or —(C₁-C₆) alkyl, each of which is optionally substituted with 1 to 3 substituents selected from the group consisting of —CF₃, —N(CH₃)₂, —CH₃, halogen, phenyl and phenyl substituted with 1 to 2 substituents selected from —CF₃ and halogen; A is ═CH₂, or CH2R*, where R* is selected from the group consisting of methylamino (—NH(CH₃)), dimethylamino (—N(CH3)2), methylethylamino (—N(CH₃)(CH₂CH₃)), methylpropylamino (—N(CH₃)(CH₂CH₂CH₃)), methylisopropylamino (—(CH₃)(CH₂(CH₃)₂), (—N(CH₃)(CH₂CH₂OH), pyrrolidine, piperidine, 4-methylpiperidine, 1-phenylmethanamine (—NCH₂Ph), and 2-phenylethanamine (—NCH₂CHPh); M is —O—, or —OC(O)—; X is hydrogen, —(C₆-C₁₀) aryl, -(5-14 membered) heteroaryl, (C₁-C₆) alkyl, -—N(CH₃)₂—(C₆-C₁₀) aryl, each of which is optionally substituted with 1 to 3 substituents selected from the group consisting of —CF₃, —N(CH₃)₂, —CH₃, halogen, phenyl and phenyl substituted with 1 to 2 substituents selected from —CF₃ and halogen. Preferably, L is —O—; Y is —(C₆-C₁₀) aryl, -(5-14 membered) heteroaryl, or —NH—(C₆-C₁₀) aryl, each of which is optionally substituted with 1 to 3 substituents selected from the group consisting of —CF₃, halogen, phenyl, and phenyl substituted with 1 to 2 substituents selected from —CF₃ and halogen; A is ═CH₂; M is —O—, or —OC(O)—; X is hydrogen, —(C₆-C₁₀) aryl, -(5-14 membered) heteroaryl, each of which is optionally substituted with 1 to 3 substituents selected from the group consisting of —CF₃, —N(CH₃)₂, —CH₃, halogen, phenyl and phenyl substituted with 1 to 2 substituents selected from —CF₃ and halogen.

In some embodiments, the present invention provides compounds of formula A1 through A45, wherein -L-Y is hydrogen, hydroxyl, azide, -(5-14 membered) heteroaryl, amino, halogen, —NH—C(O)—(C₆-C₁₀) aryl, wherein -(5-14 membered) heteroaryl is optionally substituted with 1 to 3 substituents selected from the group consisting of —CF₃, —N(CH₃)₂, —CH₃, halogen, phenyl and phenyl substituted with 1 to 2 substituents selected from —CF₃ and halogen; A is ═CH₂; M is —O—; X is hydrogen, or —C(O)—(C₆-C₁₀) aryl, wherein —C(O)—(C₆-C₁₀) aryl is optionally substituted with 1 to 3 substituents selected from the group consisting of —CF₃, —N(CH₃)₂, —CH₃, halogen, phenyl and phenyl substituted with 1 to 2 substituents selected from —CF₃ and halogen. Preferably, -L-Y is hydrogen, hydroxyl, -(5-14 membered) heteroaryl, halogen, wherein -(5-14 membered) heteroaryl is optionally substituted with 1 to 3 substituents selected from the group consisting of —CF₃, —N(CH₃)₂, —CH₃, halogen, phenyl and phenyl substituted with 1 to 2 substituents selected from —CF₃ and halogen; A is ═CH₂; X is hydrogen, or —C(O)—(C₆-C₁₀) aryl, wherein —C(O)—(C₆-C₁₀) aryl is optionally substituted with 1 to 3 substituents selected from the group consisting of —CF₃, —N(CH₃)₂, —CH₃, halogen, phenyl and phenyl substituted with 1 to 2 substituents selected from —CF₃ and halogen.

In some embodiments, the compounds provided herein correspond to the following compounds, or a salt thereof:

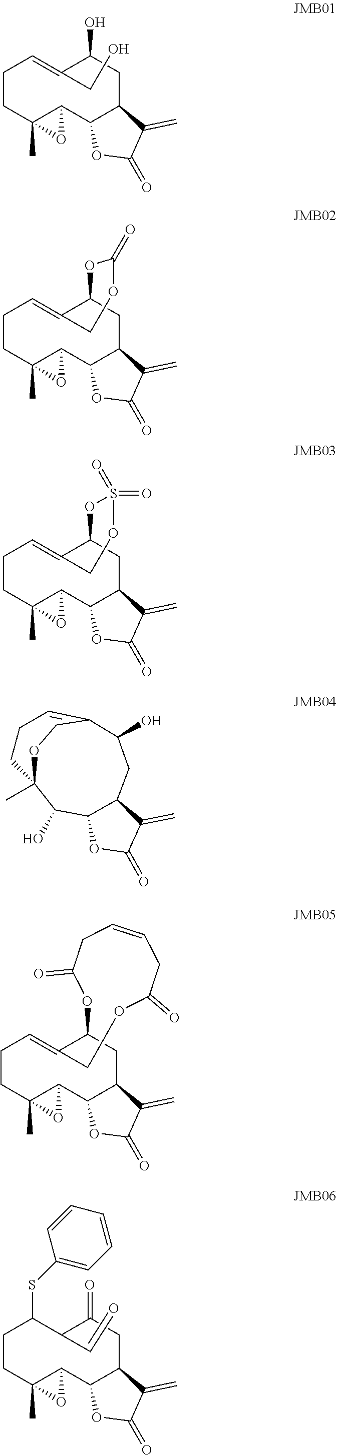

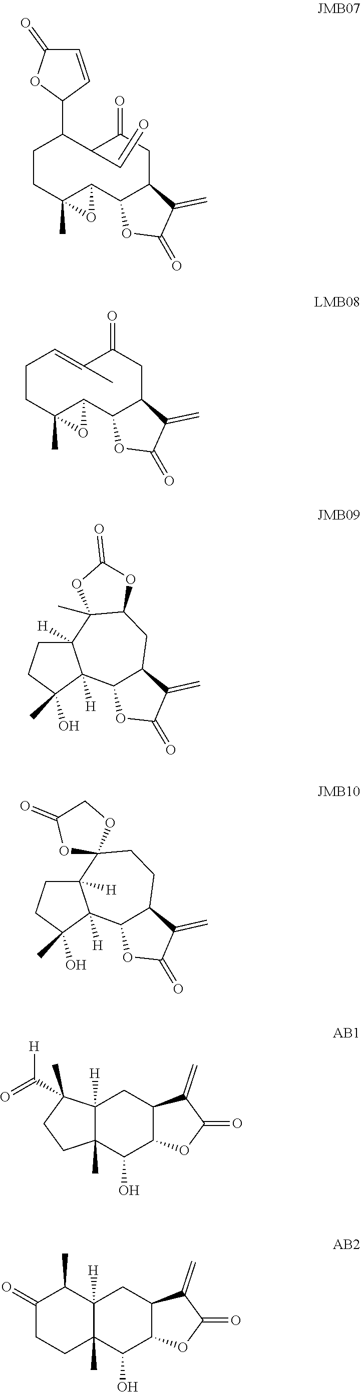

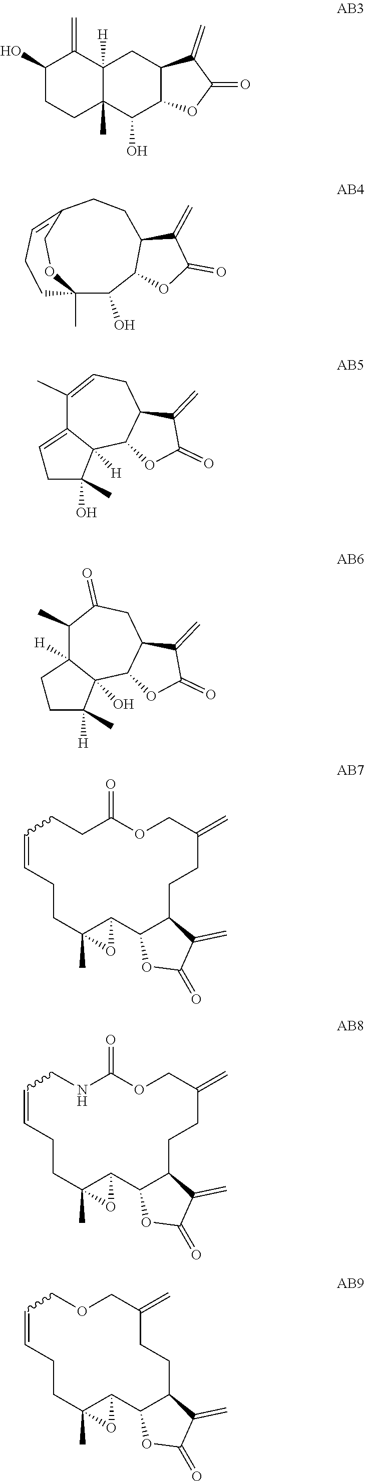

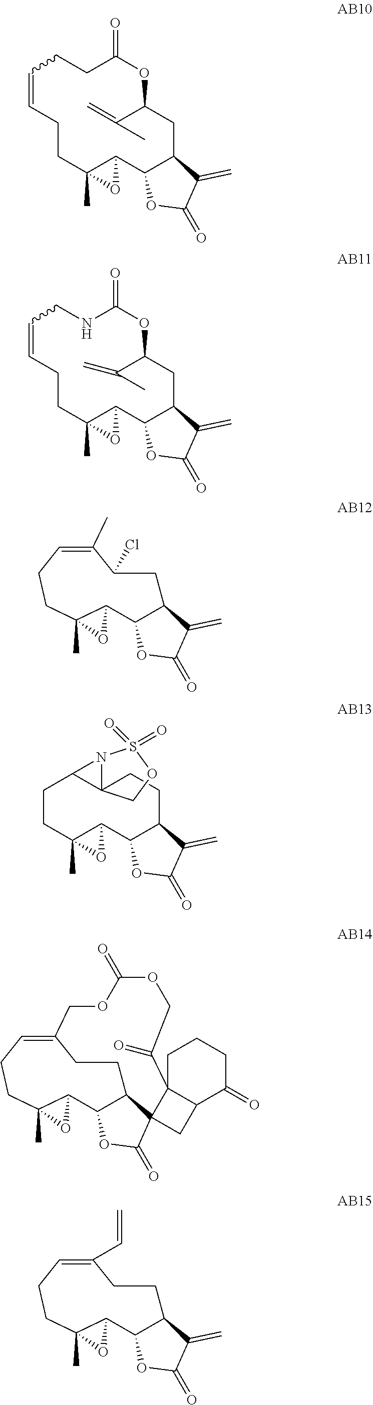

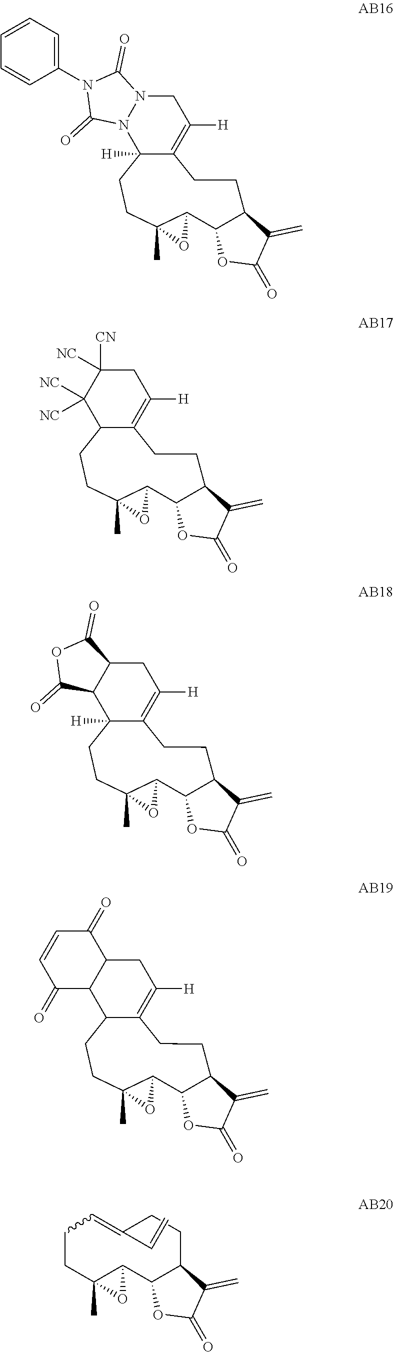 [SMILES: C=C1C(=O)O[C@@H]2[C@H]3O[C@]3(C)CC[C@@H]3C(=CCn4c(=O)n(-c5ccccc5)c(=O)n43)CC[C@@H]12 ; C=C1C(=O)O[C@@H]2[C@H]3O[C@]3(C)CCC3C(=CCC(C#N)(C#N)C3(C#N)C#N)CC[C@@H]12 ; C=C1C(=O)O[C@@H]2[C@H]3O[C@]3(C)CC[C@@H]3C(=CC[C@@H]4C(=O)OC(=O)[C@@H]43)CC[C@@H]12 ; C=C1C(=O)O[C@@H]2[C@H]3O[C@]3(C)CCC3C(=CCC4C(=O)C=CC(=O)C43)CC[C@@H]12 ; C=CC1=CCC[C@@]2(C)O[C@@H]2[C@H]2OC(=O)C(=C)[C@@H]2CC1]

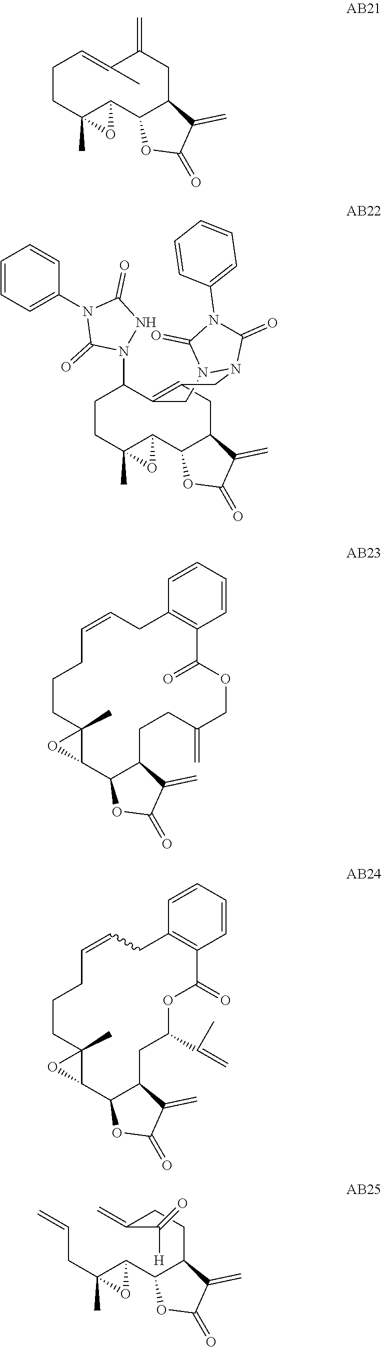

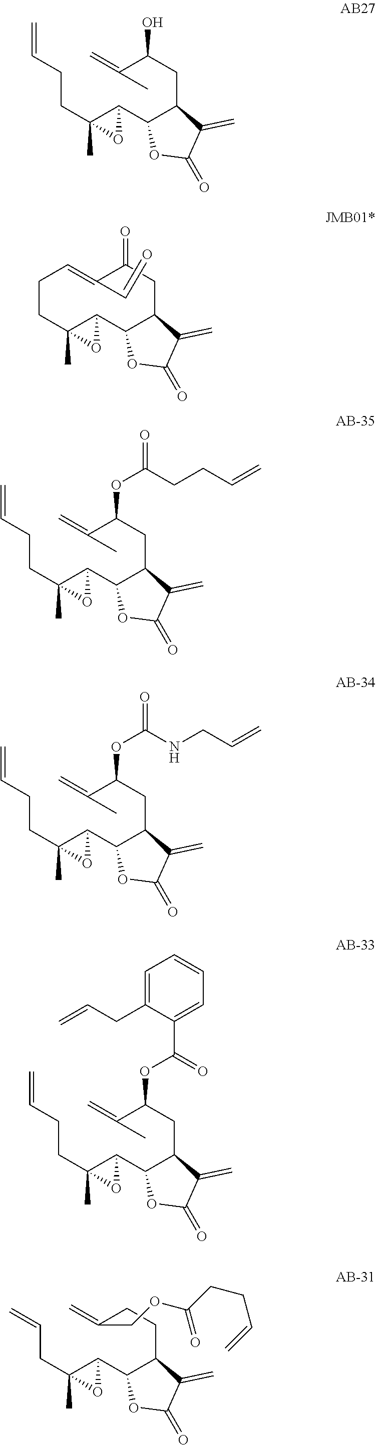

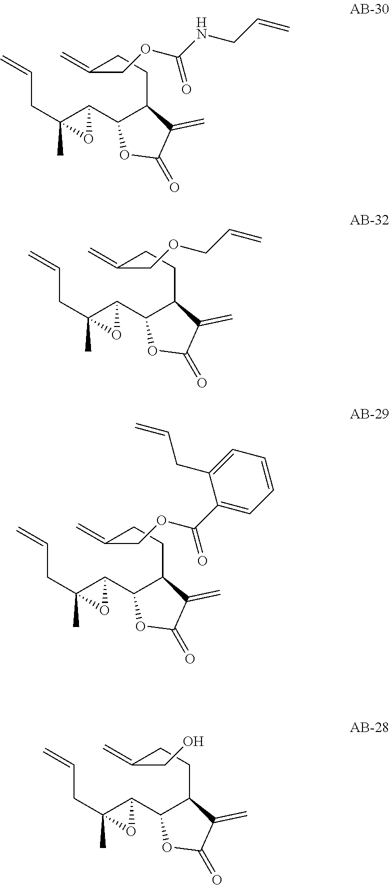

Salts of the compounds provided herein can be prepared according to standard procedures well known in the art, for example, by reacting a compound containing a one or more sufficiently basic functional group with a suitable organic or mineral acid. Similarly, base addition salts can be prepared by reacting a compound containing a one or more sufficiently acid functional group with a suitable organic or mineral base. Examples of inorganic acid addition salts includes fluoride, chloride, bromide, iodide, sulfate, nitrate, bicarbonate, phosphate, and carbonate salts. Examples of organic acid addition salts include acetate, citrate, malonate, tartrate, succinate, lactate, malate, benzoate, ascorbate, α-ketoglutarate, tosylate, and methanesulfonate salts. Examples of base addition salts include lithium, sodium, potassium, calcium, and ammonium salts.

A person skilled in the art will promptly recognize that several different chemical methods, including different chemical reagents and reaction conditions, are available for synthesizing the compounds of general formula A1 through A45, once the corresponding parent molecule, i.e., a molecule corresponding to the same formula, wherein A is ═CH₂, M=—O—, L=—O—, X=—H, and Y=—H, is made available. Accordingly, this invention focuses on the products of these transformations rather than on the specific chemical methods applied to achieve them, which of course can vary.

For example, using art-known methods, amino-adduct analogs of compounds of general formula A1 through A45, wherein A is ═CH₂, can be prepared via addition of an amine (e.g, dimethylamine, diethylamine, dipropylamine, pyrrolidine, piperidine, morpholine, and the like) to the α-methylene-g-lactone moiety via an aza-Michael addition reaction. Due to presence of a basic amino group, these amino-adduct analogs can possess improved water-solubility, which may be useful to improve the pharmacokinetic properties of these compounds.

Similarly, using art-known methods, ester analogs of compounds of general formula A1 through A45, wherein M=—O—, L=—O—, X=—H, and Y=—H, can be prepared via an esterification reaction with an activated carboxylic acid derivative, such as an acid chloride, anhydride, and the like. Similarly, using art-known methods, ether analogs of compounds of general formula A1 through A45, wherein M=—O—, L=—O—, X=—H, and Y=—H, can be prepared via an etherification reaction with an alkyl bromide in the presence of base, an O—H carbene insertion reaction in the presence of a diazo reagent, a nucleophilic substitution reaction (e.g., Mitsunobu substitution), and the like. Similarly, using art-known methods, fluorinated, chlorinated, or brominated analogs of compounds of general formula A1 through A45, wherein M=—O—, L=—O—, X=—H, and Y=—H, can be prepared via a nucleophilic substitution reaction in the presence of a deoxofluorination (e.g., DAST), bromination (e.g., PBr₃), or chlorination reagent (e.g., SOCl₂).

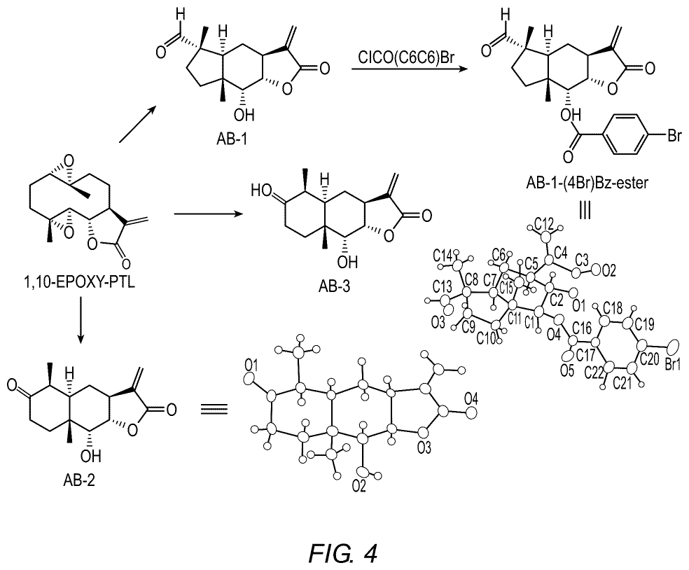

For example, as exemplified with compound AB-1-(4Br)-Bz-ester in FIG. 4, acylester derivatives of compounds of general formula A1 through A45 can be prepared via acylation with an acid chloride in dichloromethane in the presence of a weakly nucleophilic base (e.g., triethylamine, triisopropylamine, or pyridine). Alternatively, such ester derivatives can be prepared via reaction with a free acid in dichloromethane in presence of a coupling reagent (e.g., dicyclohexylcarbodiimide or DCC, O-(Benzotriazol-1-yl)-N,N,N′,N′-tetramethyluronium hexafluorophosphate or HBTU), and weakly nucleophilic base (e.g., triethylamine, triisopropylamine, or pyridine). Optionally, coupling catalysts (e.g., 4-dimethylamino-pyridine or DMAP, 1-Hydroxybenzotriazole or HOBt) can be added to facilitate the esterification reaction.

### 4.6 Use of Compounds for Treatment of Cancer and Other Diseases

The invention also provides a pharmaceutical composition comprising an effective amount of a compound of general formula A1 through A45, or a pharmaceutically acceptable salt, ester or prodrug thereof, in combination with a pharmaceutically acceptable diluent or carrier. The composition may comprise any of the preferred embodiments of these compounds described above. The pharmaceutical compositions may additionally comprise an ingredient selected from a group consisting of pharmaceutically acceptable carriers, pharmaceutically acceptable diluents and pharmaceutically acceptable excipients.

The invention also provides a method of inhibiting cancer cell growth and metastasis of cancer cells, comprising administering to a mammal afflicted with cancer, an amount of a compound of general formula A1 through A45, effective to inhibit the growth of the cancer cells. The composition may comprise any of the preferred embodiments of these compounds described above.

The invention also provides a method comprising inhibiting cancer cell growth by contacting the cancer cell in vitro or in vivo with an amount of a compound of formula A1 through A45, effective to inhibit the growth of the cancer cell. The method may also use any of the preferred embodiments of these compounds described above.

The invention also provides a compound of general formulae A1 through A45 for use in medical therapy (preferably for use in treating cancer, e.g., solid tumors), as well as the use of such compound for the manufacture of a medicament useful for the treatment of cancer and other diseases/disorders described herein. The compound may comprise any of the preferred embodiments of these compounds described above.

In one embodiment, the invention provides a method for inhibiting cancer cell growth. In one embodiment, the method comprises administering to a mammal afflicted with cancer an amount of a compound of the invention effective to inhibit the growth of the cancer cells. In one embodiment, the method comprises contacting the cancer cell in vitro or in vivo with an amount of a compound of the invention effective to inhibit the growth of the cancer cell.

In one embodiment, invention provides a method for treating bone marrow for human bone marrow transplant treatment of leukemia in a patient. In one embodiment, the method comprises treating bone marrow with a compound of the invention prior to reintroducing bone marrow into the patient.

In one embodiment, the invention provides a method for inhibiting cancer cell growth in a mammal, the method comprising the step of administering to the mammal afflicted with cancer an amount any of the disclosed compounds effective to inhibit the growth of the cancer cells. The method may be used where the cancer is acute myeloid leukemia (AML), chronic myeloid leukemia (CML), acute lymphoblastic leukemia (ALL), mantle cell lymphoma (MCL), or large B-cell lymphoma. The cancer may also be a prostate cancer, a brain cancer, a neuroblastoma, a lung cancer, a breast cancer, a skin cancer, a cervical cancer, a colon cancer, an ovary cancer, osteosarcoma or a pancreatic cancer. Specifically, the method may, as an embodiment, use a compound selected from the group consisting of: AB24, AB25, AB10, JMB08, AB16, AB19, JMB03, AB20, AB26, AB08, AB24, JMB02 AB16, AB19, AB17, AB18, AB25, AB07, JMB06 and JMB07.

In one embodiment, the invention provides for a method for treating an inflammatory condition in a mammal, the method comprising the step of administering to the mammal in need thereof, an amount of a compound disclosed above effective to reduce, prevent, or control the condition. The mammal may be human. In another embodiment, the invention provides a method for inhibiting angiogenesis in a patient in need thereof, the method comprising the step of administering to the patient an effective amount of a compound disclosed above.

In one embodiment, the invention provides for a method for treating a patient with a tumor, comprising the step of administering to the patient an effective amount of any of the disclosed compounds. The tumor may be cancerous, and may preferably be a prostate cancer, a brain cancer, a neuroblastoma, a lung cancer, a breast cancer, a skin cancer, a cervical cancer, a colon cancer, an ovary cancer, or a pancreatic cancer.

In specific embodiments, the compound may be selected from the group consisting of: AB24, AB25, AB10, JMB08, AB16, AB19, JMB03, AB20, AB26, AB08, AB24, JMB02 AB16, AB19, AB17, AB18, AB25, AB07, JMB06 and JMB07.

The following are non-limiting examples of cancers that can be treated by the disclosed methods and compositions: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma; Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocytoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

In one embodiment, the cancer is acute myeloid leukemia (AML), chronic myeloid leukemia (CML), acute lymphoblastic leukemia (ALL), mantle cell lymphoma (MCL), or large B-cell lymphoma.

In one embodiment, the cancer is prostate cancer, a brain cancer, a neuroblastoma, a lung cancer, a breast cancer, a skin cancer, a cervical cancer, a colon cancer, an ovary cancer, or a pancreatic cancer.

The invention further provides methods of treating inflammatory diseases and disorders, including, for example, rheumatoid arthritis, osteoarthritis, allergies (such as asthma), and other inflammatory conditions, such as pain (such as migraine), swelling, fever, psoriasis, inflammatory bowel disease, gastrointestinal ulcers, cardiovascular conditions, including ischemic heart disease and atherosclerosis, partial brain damage caused by stroke, skin conditions (eczema, sunburn, acne), leukotriene-mediated inflammatory diseases of lungs, kidneys, gastrointestinal tract, skin, prostatitis and paradontosis.

The invention further provides methods of treating immune response disorders, whereby the immune response is inappropriate, excessive or lacking. Such disorders include allergic responses, transplant rejection, blood transfusion reaction, and autoimmune disorders including, but not limited to, Addison's disease, alopecia areata, antiphospholipid antibody syndrome (aPL), autoimmune hepatitis, celiac disease—sprue (gluten-sensitive enteropathy), dermatomyositis, Graves' disease, Guillain-Barre syndrome, Hashimoto's disease, hemolytic anemia, idiopathic thrombocytopenic purpura, inflammatory bowel disease (IBD), inflammatory myopathies, multiple sclerosis, myasthenia gravis, pernicious anemia, primary biliary cirrhosis, psoriasis, reactive arthritis, rheumatoid arthritis, scleroderma, Sjögren's syndrome, systemic lupus erythematosus, Type I diabetes and vitiligo.

The compounds disclosed herein are useful for treating cancer. Cancers treatable by the present therapy include the solid and hematological tumors, such as leukemia, breast cancer, lung cancer, prostate cancer, colon cancer, bladder cancer, liver cancer, skin cancer, brain cancer, pancreas cancer, kidney cancer, and bone cancer, comprising administering to a mammal afflicted with the cancer an amount of the compound of general formula A1 through A45 effective to inhibit the viability of cancer cells of the mammal. The compound may be administered as primary therapy, or as adjunct therapy, either following local intervention (surgery, radiation, local chemotherapy) or in conjunction with another chemotherapeutic agent. Hematological cancers, such as the leukemias are disclosed in the Mayo Clinic Family Health Book, D. E. Larson, ed., William Morrow, N.Y. (1990) and include CLL, ALL, CML and the like. Compounds of the present invention may be used in bone marrow transplant procedure to treat bone marrow prior to reintroduction to the patient. In addition, the compounds of the present invention may be used as chemotherapy sensitizers or radiation therapy sensitizers. Accordingly, a patient, or cells, or tissues, derived from a cancer patient, are pre-treated with the compounds prior to standard chemotherapy or radiation therapy.

Within another aspect of the present invention, methods are provided for inhibiting angiogenesis in patients with non-tumorigenic, angiogenesis-dependent diseases, comprising administering a therapeutically effective amount of a composition comprising a compound of general formula A1 through A45 to a patient with a non-tumorigenic angiogenesis-dependent disease, such that the formation of new blood vessels is inhibited. Within other aspects, methods are provided for inhibit reactive proliferation of endothelial cells or capillary formation in non-tumorigenic, angiogenesis-dependent diseases, such that the blood vessel is effectively occluded. Within one embodiment, the anti-angiogenic composition comprising a compound of general formula A1 through A45 is delivered to a blood vessel which is actively proliferating and nourishing a tumor.

The magnitude of a prophylactic or therapeutic dose of the compound of general formula A1 through A45, an analog thereof or a combination thereof, in the acute or chronic management of cancer, i.e., prostate or breast cancer, will vary with the stage of the cancer, such as the solid tumor to be treated, the chemotherapeutic agent(s) or other anticancer therapy used, and the route of administration. The dose, and perhaps the dose frequency, will also vary according to the age, body weight, and response of the individual patient. In general, the total daily dose range for the compound or its analogs, for the conditions described herein, is from about 0.5 mg to about 2500 mg, in single or divided doses. Preferably, a daily dose range should be about 1 mg to about 100 mg, in single or divided doses, most preferably about 5-50 mg per day. In managing the patient, the therapy should be initiated at a lower dose and increased depending on the patient's global response. It is further recommended that infants, children, patients over 65 years, and those with impaired renal or hepatic function initially receive lower doses, and that they be titrated based on global response and blood level. It may be necessary to use dosages outside these ranges in some cases. Further, it is noted that the clinician or treating physician will know how and when to interrupt, adjust or terminate therapy in conjunction with individual patient response. The terms “an effective amount” or “an effective sensitizing amount” are encompassed by the above-described dosage amounts and dose frequency schedule.

Any suitable route of administration may be employed for providing the patient with an effective dosage of the compound (e.g., oral, sublingual, rectal, intravenous, epidural, intrathecal, subcutaneous, transcutaneous, intramuscular, intraperitoneal, intracutaneous, inhalation, transdermal, nasal spray, nasal gel or drop, and the like). While it is possible that, for use in therapy, a compound of general formula A1 through A45 may be administered as the pure chemicals, as by inhalation of a fine powder via an insufflator, it is preferable to present the active ingredient as a pharmaceutical formulation. The invention thus further provides a pharmaceutical formulation comprising a compound of general formula A1 through A45, together with one or more pharmaceutically acceptable carriers therefor and, optionally, other therapeutic and/or prophylactic ingredients. The carrier(s) must be ‘acceptable’ in the sense of being compatible with the other ingredients of the formulation and not deleterious to the recipient thereof, such as a human patient or domestic animal.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical (including buccal, rectal, vaginal and sublingual) administration. The formulations may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

Pharmaceutical formulations suitable for oral administration may be presented as discrete unit dosage forms such as hard or soft gelatin capsules, cachets or tablets each containing a predetermined amount of the active ingredient; as a powder or as granules; as a solution, a suspension or as an emulsion; or in a chewable base such as a synthetic resin or chicle for ingestion of the agent from a chewing gum. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art, i.e., with enteric coatings.

Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds according to the invention may also be formulated for parenteral administration (e.g., by injection, for example, bolus injection or continuous infusion) and may be presented in unit dose form in ampules, pre-filled syringes, small volume infusion containers or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds may be formulated as ointments, creams or lotions, or as the active ingredient of a transdermal patch. Suitable transdermal delivery systems are disclosed, for example, in Fisher et al. U.S. Pat. No. 4,788,603, or Bawa et al. U.S. Pat. Nos. 4,931,279; 4,668,506 and 4,713,224. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described formulations can be adapted to give sustained release of the active ingredient employed, e.g., by combination with certain hydrophilic polymer matrices, e.g., comprising natural gels, synthetic polymer gels or mixtures thereof. The polymer matrix can be coated onto, or used to form, a medical prosthesis, such as a stent, valve, shunt, graft, or the like.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of the active compound with the softened or melted carrier(s) followed by chilling and shaping in molds.

Formulations suitable for vaginal administration may be presented as pessaries, tampons, creams, gels, pastes, foams or sprays containing, in addition to the active ingredient, such carriers as are known in the art to be appropriate.

For administration by inhalation, the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds according to the invention may take the form of a dry powder composition, for example, a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

For intra-nasal administration, the compounds provided herein may be administered via a liquid spray, such as via a plastic bottle atomizer. Typical of these are the Mistometer® (Wintrop) and the Medihaler® (Riker).

For topical administration to the eye, the compounds can be administered as drops, gels (U.S. Pat. No. 4,255,415), gums (see U.S. Pat. No. 4,136,177) or via a prolonged-release ocular insert.

The term “treatment” refers to any treatment of a pathologic condition in a mammal, particularly a human, and includes: (i) preventing the pathologic condition from occurring in a subject which may be predisposed to the condition but has not yet been diagnosed with the condition and, accordingly, the treatment constitutes prophylactic treatment for the disease condition; (ii) inhibiting the pathologic condition, i.e., arresting its development; (iii) relieving the pathologic condition, i.e., causing regression of the pathologic condition; or (iv) relieving the conditions mediated by the pathologic condition.

The term “therapeutically effective amount” refers to that amount of a compound of the invention that is sufficient to effect treatment, as defined above, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “pharmaceutically acceptable salts” includes, but is not limited to, salts well known to those skilled in the art, for example, mono-salts (e.g., alkali metal and ammonium salts) and poly salts (e.g., di- or tri-salts,) of the compounds of the invention. Pharmaceutically acceptable salts of compounds of formulas A1 through A45 are where, for example, an exchangeable group, such as hydrogen in —OH, —NH—, or —P(═O)(OH)—, is replaced with a pharmaceutically acceptable cation (e.g., a sodium, potassium, or ammonium ion) and can conveniently be prepared from a corresponding compound of formula A1 through A45 by, for example, reaction with a suitable base. Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example, calcium) salts of carboxylic acids can also be made.

The compounds provided herein may contain one or more chiral centers.

The compounds may include racemic mixtures, diastereomers, enantiomers, and/or mixture enriched in one or more stereoisomer. When a group of substituents is disclosed herein, all the individual members of that group and all subgroups, including any isomers, enantiomers, and diastereomers are intended to be included in this disclosure. Additionally, all isotopic forms of the compounds disclosed herein are intended to be included in this disclosure. For example, it is understood that any one or more hydrogens in a molecule disclosed herein can be replaced with deuterium or tritium.

A person skilled in the art will also appreciate that starting materials, biological materials, reagents, synthetic methods, purification methods, analytical methods, assay methods, and biological methods other than those specifically exemplified can be employed in the practice of the invention. All art-known functional equivalents of any such materials and methods are intended to be included in the invention.

Unless defined otherwise herein, all technical and scientific terms used herein have the same meaning as commonly understood by one of ordinary skill in the art to which this disclosure pertains.

Citation to any reference herein does not constitute an admission that the reference constitutes prior art.

The following examples are offered by way of illustration and not by way of limitation.

### 5. EXAMPLES

### 5.1 Example 1: Synthesis of Complex Natural Product-Like Compounds

This example describes and demonstrates the preparation of compounds of general formula A1 through A45 via chemoenzymatic scaffold rearrangement/editing of parthenolide. Specifically, this example demonstrates the preparation of compounds of general formula A1 through A45 wherein A is ═CH₂, M=—O—, L=—O—, X=—H, and Y=—H. Schematic representations of the synthetic routes used for the preparation of these compounds are shown in FIGS. 1-7.

Enzymatic synthesis of 14-hydroxy-parthenolide (14-OH-PTL), 9(S)-hydroxy-parthenolide (9-OH-PTL), and 1,10-epoxy parthenolide. The oxyfunctionalized parthenolide derivatives 14-hydroxy-parthenolide (14-OH-PTL), 9(S)-hydroxy-parthenolide (9-OH-PTL), and 1,10-epoxy parthenolide were prepared via enzymatic oxidation of parthenolide using engineered variant of cytochrome P450 BM3 (also known as CYP102A1), according to reported methods (Kolev et al., ACS Chem Biol. (2014); 9(1):164-73).

General conditions for rearrangement of 14-OH parthenolide, 9(S)—OH parthenolide, and 1,10-epoxy parthenolide with tosylic acid: to a round-bottomed flask p-Toluenesulfonic acid monohydrate (0.35 eq.) was suspended in benzene (30 mL) and heated to reflux. Upon dissolution of the solid, the solution stirred at reflux for an additional 15 minutes then allowed to cool slowly under inert atmosphere, resulting in formation of a crystalline slurry. The slurry was concentrated in vacuo, then resuspended in DCM (wet). To the slurry 14-OH PTL, 9(S)—OH PTL, or 1,10-Epoxy PTL was slowly added as a solution in DCM (wet) over 1 minute while stirring at room temperature. The reaction was allowed to stir at room temperature for 16 hours before confirming consumption of starting material by TLC. Reaction was quenched with saturated sodium bicarbonate, back extracted with DCM (3×), and dried over sodium sulfate. Organic layer was then concentrated in vacuo and purified by flash chromatography on silica (eluting mixture 60% EtOAc in hexanes).

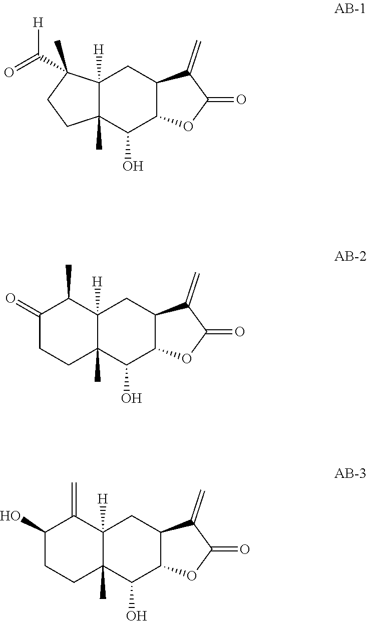 [SMILES: C=C1C(=O)O[C@H]2[C@H]1C[C@H]1[C@@](C)(CC[C@]1(C)C=O)[C@H]2O ; C=C1C(=O)O[C@H]2[C@H]1C[C@@H]1[C@H](C)C(=O)CC[C@@]1(C)[C@H]2O ; C=C1[C@H](O)CC[C@]2(C)[C@@H]1C[C@H]1C(=C)C(=O)O[C@@H]1[C@@H]2O]

AB-1-(48r)Bz-ester: A solution of A-1 (6.0 mg, 0.022 mmol) in dry DCM (2 mL) was stirred while cooled by ice bath, and then charged with triethylamine (5 eq.), DMAP (cat.), and p-bromobenzoyl chloride while stirring. Reaction was allowed to warm to room temperature over 1 hour, then stirred for an additional 6 hours before quenching with saturated sodium bicarbonate. Aqueous layer was back extracted (3× DCM), then the crude organics combined and washed with brine. Organic layer was then filtered and concentrated to a residue and purified by flash chromatography (eluting with 20% EtOAc in hexanes) to afford P55 8.8 mg, 88%) as an oil which crystallized upon standing. ¹H NMR (500 MHz, CDCl₃) δ 9.42 (s, 1H), 7.90 (d, J=8.6 Hz, 2H), 7.58 (d, J=8.6 Hz, 2H), 6.12 (d, J=3.3 Hz, 1H), 5.47 (d, J=3.0 Hz, 1H), 5.27 (d, J=10.3 Hz, 1H), 4.20-4.12 (m, 1H), 2.82 (ddddd, J=11.0, 11.0, 3.4, 3.4, 3.4 Hz, 1H), 2.19-2.09 (m, 2H), 1.86 (ddd, J=12.7, 3.3, 3.3 Hz, 1H), 1.77-1.66 (m, 2H), 1.64-1.53 (m, 2H), 1.48 (dd, J=12.3, 12.2 Hz, 1H), 1.19 (s, 3H), 1.17 (s, 3H). ¹³C NMR (126 MHz, CDCl₃) δ 170.0, 155.9, 143.5, 138.3, 124.0, 116.0, 81.8, 75.6, 64.4, 62.0, 40.4, 38.3, 34.5, 32.6, 29.9, 25.5, 23.4, 18.8, 18.2, 14.9. HRMS (ESI) m/z calculated for C₂₂H₂₄BrO₅⁺ [M+H]⁺: 447.0802, found: 447.0797.

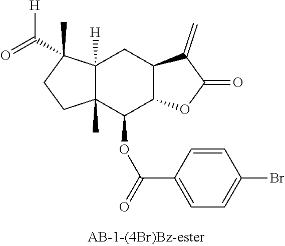 [SMILES: C=C1C(=O)O[C@@H]2[C@@H](OC(=O)c3ccc(Br)cc3)[C@]3(C)CC[C@](C)(C=O)[C@H]3C[C@@H]12]

AB-1, AB-2, and AB-3: standard procedure was applied for rearrangement of 1,10-epoxy PTL (170 mg, 0.640 mmol) with tosylic acid (42 mg, 0.22 mmol) to afford the product AB-1 (42 mg, 25%) as a clear oil and products AB-2 (54 mg, 32%), and AB-3 (18 mg, 11%) as crystalline solids. AB-1: ¹H NMR (500 MHz, CDCl₃) δ 9.38 (s, 1H), 6.05 (d, J=3.2 Hz, 1H), 5.40 (d, J=3.1 Hz, 1H), 3.87 (dd, J=12.0, 10.1 Hz, 1H), 3.67 (d, J=9.8 Hz, 1H), 2.62 (ddd, J=12.8, 11.7, 3.4 Hz, 1H), 2.12 (dd, J=14.5, 8.9 Hz, 1H), 2.01 (s, 1H), 1.90 (ddd, J=12.9, 8.0, 5.3 Hz, 3H), 1.79-1.69 (m, 3H), 1.45-1.31 (m, 3H), 1.25-1.17 (m, 1H), 1.15 (s, 3H), 1.03 (s, 3H). ¹³C NMR (126 MHz, CDCl₃) δ 203.1, 138.5, 117.8, 82.9, 79.0, 52.0, 50.5, 49.0, 48.2, 37.4, 33.5, 29.7, 20.8, 18.7, 14.1. HRMS (ESI) m/z calculated for C₁₆H₂₃O₄Na⁺ [M+MeOH+Na]⁺: 319.1521, found: 319.1513. AB-2: ¹H NMR (500 MHz, CDCl₃): δ 6.09 (d, J=3.2 Hz, 1H), 5.44 (d, J=3.1 Hz, 1H), 3.96 (dd, J=11.4, 9.9 Hz, 1H), 3.54 (d, J=9.9 Hz, 1H), 2.61-2.28 (m, 5H), 2.07 (ddd, J=13.0, 3.4, 3.4 Hz, 1H), 1.56 (ddd, J=14.1, 13.8, 5.6 Hz, 1H), 1.49 (ddd, J=12.0, 12.0, 3.5 Hz, 1H), 1.36 (q, J=11.9 Hz, 1H), 1.17 (s, 3H), 1.05 (d, J=6.5 Hz, 3H). ¹³C NMR (126 MHz, CDCl₃): δ 212.1, 170.7, 139.2, 118.8, 83.1, 79.5, 51.6, 47.2, 45.1, 41.8, 37.5, 37.1, 25.7, 12.7, 12.1 HRMS (ESI) m/z calculated for C₁₅H₂₀O₄Na⁺ [M+Na]⁺: 287.1260, found: 287.1251. AB-3: ¹H NMR (500 MHz, CDCl₃): δ 6.11 (d, J=3.2 Hz, 1H), 5.47 (d, J=3.1 Hz, 1H), 5.18 (s, 1H), 4.73 (s, 1H), 4.02 (dd, J=11.7, 5.7 Hz, OH), 3.91 (dd, J=11.4, 9.8 Hz, 1H), 3.60 (d, J=9.9 Hz, 1H), 2.61-2.52 (m, 1H), 2.10 (ddd, J=13.3, 3.3, 3.3 Hz, 1H), 2.08-1.89 (m, 3H), 1.61 (q, J=12.4 Hz, 1H), 1.54-1.43 (m, 1H), 1.39 (ddd, J=13.4, 13.3, 3.6 Hz, 1H), 0.80 (s, 3H). ¹³C NMR (126 MHz, CDCl₃): δ 170.7, 150.8, 139.4, 118.8, 105.4, 83.4, 79.9, 73.55, 49.1, 47.0, 43.6, 36.0, 32.5, 24.7, 12.6. HRMS (ESI) m/z calculated for C₁₅H₂₀O₄Na⁺ [M+Na]⁺: 287.1260, found: 287.1253.

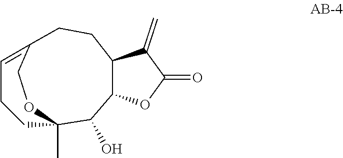 [SMILES: C=C1C(=O)O[C@H]2[C@H]1CCC1=CCC[C@](C)(OC1)[C@H]2O]

AB-4: To a solution of 14-OH parthenolide (8 mg, 0.03 mmol) in DCM (2 ml, dry) boron tribromide (0.7 eq., 0.021 mmol) was slowly added as a stock solution in 0.5 mL dry DCM at room temperature over 1 minute. Reaction was allowed to stir at room temperature for 16 hours before confirming consumption of starting material by TLC. Reaction was quenched with saturated sodium bicarbonate, back extracted with DCM (3×), and dried over sodium sulfate. Organic layer was then concentrated in vacuo and purified by flash chromatography on silica (eluting mixture 30% EtOAc in hexanes) to afford AB-4 (2.8 mg, 35%) as a clear oil. ¹H NMR (CDCl₃): δ 6.30 (d, J=3.7 Hz, 1H), 5.64 (br dd, J=7.9, 1.8 Hz, 1H), 5.59 (d, J=3.3 Hz), 4.54 (d, J=12.15 Hz, 1H), 4.52 (dd, J=6.6, 6.6 Hz), 4.51 (br s, 1H), 3.58 (dd, J=12.0, 1.8 Hz, 1H), 2.71 (dddd, J=16.9, 9.9, 3.2, 3.2 Hz 1H), 2.63 (br dddd, J=7.9, 4.0, 4.0, 3.7 Hz), 2.48 (s, 1 OH), 2.37 (m, 1H), 2.31 (dddd, J=17.8, 10.9, 7.5, 7.5 Hz, 1H), 2.20 J=15.7, 10.3, 7.0 Hz, 1H), 2.09 (m, 4H), 1.14 (s, 3H). ¹³C NMR (126 MHz, CDCl₃) δ 169.5, 140.7, 139.8, 131.6, 121.6, 82.0, 80.9, 78.2, 64.2, 49., 38.3, 33.0, 29.6, 25.9, 23.4. HRMS (ESI) m/z calculated for C₁₅H₂₀O₄Na⁺ [M+Na]⁺: 287.1260, found: 287.1252.

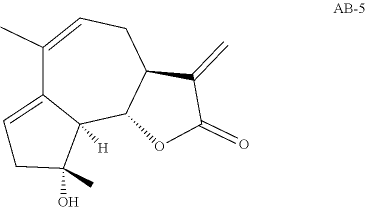 [SMILES: C=C1C(=O)O[C@H]2[C@H]1CC=C(C)C1=CC[C@@](C)(O)[C@@H]12]

AB-5: To a solution of 9(S)—OH parthenolide (10 mg, 0.038 mmol) in dry DCM (10 ml) bismuth (III) triflate (25 mg, 1 eq., 0.038 mmol) was added in a single portion, resulting in a solution that gradually turned purple, and the reaction was allowed to stir to room temperature overnight. Reaction was allowed to stir at room temperature for 16 hours before sampling for TLC before confirming consumption of starting material by TLC. Reaction was quenched with saturated sodium bicarbonate, back extracted with DCM (3×), and dried over sodium sulfate. Organic layer was then concentrated in vacuo and purified by flash chromatography on silica (eluting mixture 30% EtOAc in hexanes) to afford AS-5 (4.5 mg, 46%) as a solid. ¹H NMR (500 MHz, CDCl3) δ 6.26 (d, J=3.4 Hz, 1H), 5.92 (dd, J=2.9, 2.9 Hz, 1H), 5.56 (d, J=7.8 Hz, 1H), 5.54 (d, J=3.1 Hz, 1H), 4.16 (dd, J=11.6, 9.5 Hz, 1H), 3.08 (ddd, J=11.6, 2.7, 2.7 Hz, 1H), 2.85 (ddd, J=15.8, 8.1, 3.5 Hz, 1H), 2.79-2.72 (m, 1H), 2.70 (d, J=17.1 Hz, 1H), 2.39 (dd, J=17.1, 3.6 Hz, 1H), 2.17-2.07 (m, 1H), 1.96-1.92 (m, 3H), 1.41 (s, 3H). ¹³C NMR (126 MHz, CDCl3) δ 139.3, 138.3, 131.2, 124.3, 121.0, 83.8, 83.0, 62.6, 54.1, 46.5, 46.3, 30.6, 30.4, 25.7, 24.6. HRMS (ESI) m/z calculated for C15H18O3Na+[M+Na]+: 269.1154, found: 269.1148.

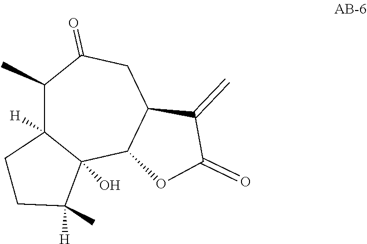 [SMILES: C=C1C(=O)O[C@H]2[C@H]1CC(=O)[C@H](C)[C@@H]1CC[C@H](C)[C@@]12O]

AB-6: To solution of 9(S)—OH parthenolide (20 mg, 0.076 mmol) dissolved in dry DCM (40 ml) a stock solution of boron trifluoride diethyl etherate (0.5 eq., 0.038 mmol) in 0.1 mL dry DCM was added in a single portion, resulting in the gradual formation of a purple solution. Reaction was allowed to stir at room temperature for 4 hours before confirming consumption of starting material by TLC. Reaction was quenched with saturated sodium bicarbonate, back extracted with DCM (3×), and dried over sodium sulfate. Organic layer was then concentrated in vacuo and dry-loaded onto silica for purification by flash chromatography on silica (eluting mixture 30% EtOAc in hexanes) to afford AB-6 (2.1 mg, 11%) as a solid. ¹H NMR: (500 MHz, CDCl₃) δ 6.26 (d, J=2.7 Hz, 1H), 5.49 (d, J=2.4 Hz, 1H), 4.01-3.93 (m, 2H), 3.10 (dq, J=10.7, 6.7 Hz, 1H), 2.94 (dd, J=18.0, 3.4 Hz, 1H), 2.49-2.37 (m, 1H), 2.22-2.12 (m, 2H), 1.87-1.79 (m, 2H), 1.70-1.58 (m, 2H), 1.02 (d, J=6.7 Hz, 3H), 0.97 (d, J=6.8 Hz, 3H). ¹³C NMR (126 MHz, CDCl₃) δ 212.4, 170.3, 139.2, 121.2, 85.5, 81.1, 46.2, 45.1, 43.4, 42.5, 36.1, 30.1, 27.5, 12.0, 14.4. HRMS (ESI) m/z calculated for C₁₅H₂₀O₄Na⁺ [M+Na]⁺: 287.1260, found: 287.1252.

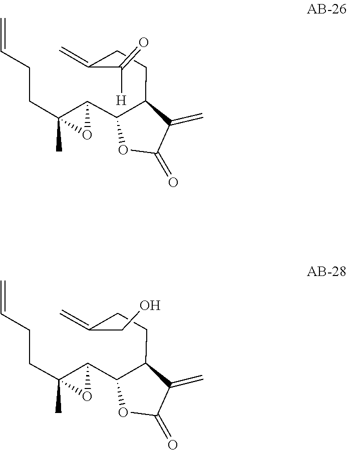

AB-28 and AB-26: To a solution of 14-OH parthenolide (30 mg, 0.11 mmol) in dichloromethane (dry) ethylene (1 atm) was sparged for 10 minutes. To the solution Hoveyda-Grubbs Catalyst M720 (Hoveyda-Grubbs Catalyst 2nd Generation, 0.2 eq., 0.023 mmol, 14 mg) was added as a solid in a single portion, and reaction was sparged for a 1 minute before sealing the reaction under ethylene (1 atm). Reaction was stirred for 4 hours then sparged with argon for 10 minutes. Reaction was evaporated in vacuo and purified by two rounds of silica chromatography (eluting mixture 30% EtOAc in hexanes) to afford AB-28 (21 mg, 63%) and AB-26 (1.8 mg, 6%) as oils. AB-26: ¹H NMR (500 MHz, CDCl₃) δ 9.53 (s, 1H), 6.35 (d, J=2.4 Hz, 1H), 6.27 (s, 1H), 6.06 (s, 1H), 5.76 (ddd, J=22.3, 11.3, 7.7 Hz, 1H), 5.71 (d, J=2.1 Hz, 1H), 5.03 (d, J=17.2 Hz, 1H), 4.97 (d, J=10.2 Hz, 1H), 4.15 (dd, J=8.0, 4.0 Hz, 1H), 2.92 (td, J=6.5, 3.6 Hz, 1H), 2.73 (d, J=8.0 Hz, 1H), 2.28 (t, J=8.2 Hz, 2H), 2.23-2.05 (m, 2H), 1.83-1.75 (m, 2H), 1.73-1.64 (m, 1H), 1.51 (ddd, J=13.9, 9.3, 7.1 Hz, 1H), 1.38 (s, 3H). ¹³C NMR (126 MHz, CDCl₃) δ 194.8, 170.1, 149.5, 138.2, 138.0, 135.5, 124.2, 116.1, 82.1, 64.6, 61.4, 42.9, 38.3, 32.3, 30.1, 25.4, 18.3. HRMS (ESI) m/z calculated for C₁₇H₂₅O₄⁺ [M+H]⁺: 293.1748, found: 293.1746. AB-28: ¹H NMR (500 MHz, CDCl₃) δ 6.31 (s, 1H), 5.74 (ddd, J=13.3, 9.9, 5.7 Hz, 1H), 5.66 (s, 1H), 5.06 (s, 1H), 5.02 (d, J=17.1 Hz, 1H), 4.96 (d, J=10.2 Hz, 1H), 4.85 (s, 1H), 4.11 (dd, J=8.1, 3.7 Hz, 1H), 4.05 (s, 2H), 2.97-2.89 (m, 1H), 2.72 (d, J=8.1 Hz, 1H), 2.26-1.98 (m, 4H), 1.92-1.63 (m, 4H), 1.49 (ddd, J=14.8, 8.2, 8.2 Hz, 1H), 1.36 (s, 3H), 1.23 (d, J=7.1 Hz, 1H). ¹³C NMR (126 MHz, CDCl₃) δ 190.5, 170.4, 148.1, 138.2, 124.2, 116.2, 111.6, 82.4, 66.6, 64.6, 61.4, 42.7, 38.3, 32.2, 30.1, 29.6, 18.3. HRMS (ES) m/z calculated for C₁₇H₂₅O₄⁺ [M+H]⁺: 293.1748, found: 293.1743.

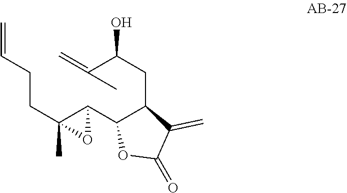 [SMILES: C=CCC[C@@]1(C)O[C@@H]1[C@H]1OC(=O)C(=C)[C@@H]1C[C@H](O)C(=C)C]

AB-27: To a solution of 9(S)—OH parthenolide (55 mg, 0.21 mmol) in dichloromethane (dry) ethylene (1 atm) was sparged for 10 minutes. To the solution Hoveyda-Grubbs Catalyst M720 (Hoveyda-Grubbs Catalyst 2nd Generation, 0.2 eq., 0.023 mmol, 14 mg) was added as a solid in a single portion, and reaction was sparged for a 1 minute before sealing the reaction under ethylene (1 atm). While stirring under ethylene (1 atm), seven additional portions Hoveyda-Grubbs Catalyst M720 (Hoveyda-Grubbs Catalyst 2nd Generation, 7×0.2 eq.) were added every 8 hours, with a total of 1.6 equivalents added over 64 hours. Reaction was evaporated in vacuo and purified by two rounds of silica chromatography (eluting mixture 30% EtOAc in hexanes) to afford AB-27 (28 mg, 47%) as an oil. ¹H NMR (500 MHz, CDCl₃) δ 6.32 (d, J=2.5 Hz, 1H), 5.75 (tdd, J=17.2, 8.8, 4.4 Hz, 1H), 5.67 (d, J=2.2 Hz, 1H), 5.03 (dd, J=17.3, 1.9 Hz, 1H), 4.99 (s, 1H), 4.97 (d, J=10.0 Hz, 1H), 4.90 (s, 1H), 4.37 (dd, J=8.0, 3.9 Hz, 1H), 4.14 (dd, J=8.6, 4.1 Hz, 1H), 3.16 (d, J=8.1 Hz, 1H), 2.74 (d, J=8.0 Hz, 1H), 2.22-2.06 (m, 2H), 1.90-1.73 (m, 2H), 1.72 (s, 3H), 1.61-1.43 (m, 3H), 1.41 (s, 3H). ¹³C NMR (126 MHz, CDCl₃) δ 170.4, 147.7, 139.0, 138.2, 123.7, 116.1, 112.4, 82.0, 73.3, 64.7, 61.6, 40.2, 39.6, 38.5, 30.1, 18.6, 18.2.

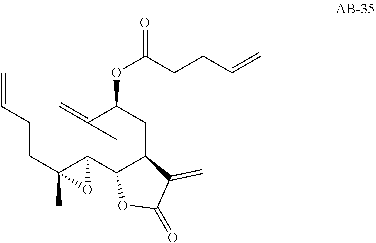 [SMILES: C=CCCC(=O)O[C@@H](C[C@H]1C(=C)C(=O)O[C@@H]1[C@H]1O[C@]1(C)CCC=C)C(=C)C]

AB-35: To a solution of AB-27 (10 mg, 0.034 mmol) in dichloromethane (3 ml, dry) triethylamine (5 eq., 24 μL, 0.172 mmol) and 4-pentenyl chloride (8 μL, 0.068 mmol, 2 eq.) were added while stirring on ice. To the reaction a catalytic amount of DMAP was added, and the reaction was allowed to stir to room temperature for 16 hours. Reaction was quenched with saturated sodium bicarbonate, back extracted with DCM (3×), and dried over sodium sulfate. Organic layer was then concentrated in vacuo and purified by flash chromatography on silica (eluting mixture 15% EtOAc in hexanes) to afford AB-35 (5.3 mg, 41%) as an oil. ¹H NMR (500 MHz, CDCl₃) δ 6.33 (d, J=2.4 Hz, 1H), 5.85-5.68 (m, 3H), 5.22 (dd, J=8.4, 5.3 Hz, 1H), 5.07-4.92 (m, 6H), 4.21 (dd, J=7.5, 3.9 Hz, 1H), 2.94 (dddt, J=8.2, 6.2, 4.4, 2.4 Hz, 1H), 2.69 (d, J=7.5 Hz, 1H), 2.45-2.39 (m, 2H), 2.35 (dddd, J=8.3, 8.3, 1.6, 1.6 Hz, 2H), 2.21-2.04 (m, 1H), 1.97-1.84 (m, 2H), 1.75-1.66 (m, 4H), 1.62-1.48 (m, 4H), 1.41 (s, 3H). ¹³C NMR (126 MHz, CDCl₃) δ 172.7, 169.9, 142.9, 138.4, 138.2, 137.1, 124.0, 116.5, 116.0, 114.9, 81.8, 75.0, 64.3, 61.9, 40.3, 38.3, 38.0, 34.3, 29.9, 29.4, 18.8, 18.2.

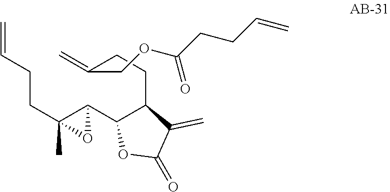 [SMILES: C=CCCC(=O)OCC(=C)CC[C@H]1C(=C)C(=O)O[C@@H]1[C@H]1O[C@]1(C)CCC=C]

AB-31: To a solution of AB-28 (5 mg, 0.014 mmol) in dichloromethane (3 ml, dry) triethylamine (3 eq., 7 μL, 0.05 mmol) and 4-pentenyl chloride (3 μL, 0.021 mmol, 1.3 eq.) were added while stirring on ice. To the reaction a catalytic amount of DMAP was added, and the reaction was allowed to stir to room temperature for 16 hours. Reaction was quenched with saturated sodium bicarbonate, back extracted with DCM (3×), and dried over sodium sulfate. Organic layer was then concentrated in vacuo and purified by flash chromatography on silica (eluting mixture 15% EtOAc in hexanes) to afford AB-35 (4.6 mg, 73%) as an oil. ¹H NMR (500 MHz, CDCl₃) δ 6.34 (d, J=2.3 Hz, 1H), 5.86-5.70 (m, 2H), 5.66 (d, J=2.1 Hz, 1H), 5.09 (s, 1H), 5.06 (ddd, J=9.9, 1.6, 1.6 Hz, 1H), 5.03 (ddd, J=10.0, 1.6, 1.6 Hz, 1H), 5.02-4.96 (m, 2H), 4.94 (s, 1H), 4.52 (s, 2H), 4.11 (dd, J=8.0, 3.7 Hz, 1H), 2.96-2.90 (m, 1H), 2.73 (d, J=8.0 Hz, 1H), 2.46-2.34 (m, 3H), 2.23-2.03 (m, 4H), 1.86-1.69 (m, 3H), 1.37 (s, 3H).

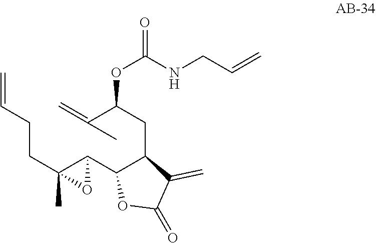 [SMILES: C=CCC[C@@]1(C)O[C@@H]1[C@H]1OC(=O)C(=C)[C@@H]1C[C@H](OC(=O)NCC=C)C(=C)C]

AB-34: To a solution of AB-27 (10 mg, 0.034 mmol) in dichloromethane (2 ml, dry) allyl isocyanate (15 eq., 45 μL, 0.51 mmol) and dibutyl tin dilaurate (10 μL, 0.017 mmol, 0.05 eq.) were added and the reaction was allowed to proceed for 40 hours. The crude reaction was then washed with saturated sodium bicarbonate followed by brine, then the aqueous layers were back extracted with DCM (3×), and combine organic layers dried over sodium sulfate. Crude material was then concentrated in vacuo and purified by flash chromatography on silica (eluting mixture 20% EtOAc in hexanes) to afford AB-34 (5.3 mg, 41%) as an oil. ¹H NMR (500 MHz, CDCl₃) δ 6.32 (d, J=2.4 Hz, 1H), 5.81 (dddd, J=22.6, 11.2, 6.0, 6.0 Hz, 2H), 5.72 (d, J=2.2 Hz, 1H), 5.29-5.07 (m, 5H), 5.04-4.93 (m, 3H), 4.79 (s, 1H), 4.23 (dd, J=7.6, 3.7 Hz, 1H), 3.78 (dd, J=5.9, 5.5 Hz, 2H), 3.00 (s, 1H), 2.71 (d, J=7.6 Hz, 1H), 2.32 (dd, J=7.6, 7.6 Hz, 1H), 2.21-2.04 (m, 2H), 1.89 (dd, J=6.8, 6.8 Hz, 2H), 1.72 (s, 3H), 1.64-1.48 (m, 2H), 1.42 (s, 3H). ¹³C NMR (126 MHz, CDCl₃) δ 178.3, 170.0, 155.9, 143.5, 138.3, 124.0, 116.0, 81.8, 75.6, 64.4, 62.0, 40.4, 38.3, 34.5, 32.6, 29.9, 25.5, 23.4, 18.8, 18.2, 14.9.

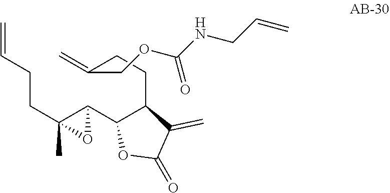 [SMILES: C=CCC[C@@]1(C)O[C@@H]1[C@H]1OC(=O)C(=C)[C@@H]1CCC(=C)COC(=O)NCC=C]

AB-34: To a solution of AB-28 (10 mg, 0.034 mmol) in dichloromethane (3 ml, dry) allyl isocyanate (5 eq., 15 μL, 0.17 mmol) and dibutyl tin dilaurate (10 μL, 0.017 mmol, 0.5 eq.) were and the reaction was allowed to proceed for 40 hours. Crude organic layers were then washed with saturated sodium bicarbonate then brine, then aqueous layers back extracted with DCM (3×), and dried over sodium sulfate. Organic layer was then concentrated in vacuo and purified by flash chromatography on silica (eluting mixture 20% EtOAc in hexanes) to afford AB-34 (6.8 mg, 58%) as an oil.

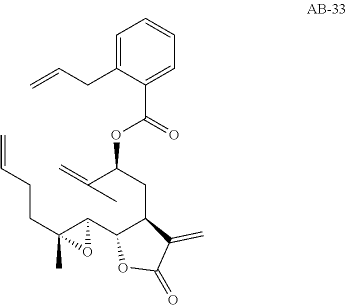 [SMILES: C=CCC[C@@]1(C)O[C@@H]1[C@H]1OC(=O)C(=C)[C@@H]1C[C@H](OC(=O)c1ccccc1CC=C)C(=C)C]

AB-33: To a solution of AB-27 (5 mg, 0.017 mmol) in dichloromethane (2 ml, dry) 2-allyl benzoic acid (10 eq., 22 mg, 0.17 mmol), N,N′-Dicyclohexylcarbodiimide (3 eq., 11 mg, 0.05 mmol), and catalytic DMAP were added while stirring. Reaction was stirred at room temperature for 16 hours, solvent was removed in vacuo and the crude reaction resuspended in carbon tetrachloride, then filtered to remove the DCU by-product. The crude material was then purified by flash chromatography on silica (eluting mixture 15% EtOAc in hexanes) to afford AB-33 (5.5 mg, 72%) as a solid. ¹H NMR (500 MHz, CDCl₃) δ 7.87 (dd, J=8.1, 1.5 Hz, 1H), 7.53-7.35 (m, 2H), 7.34-7.26 (m, 2H), 6.33 (d, J=2.5 Hz, 1H), 5.98 (dddd, J=16.6, 10.1, 6.3, 6.3 Hz, 1H), 5.76 (d, J=2.1 Hz, 1H), 5.70 (dddd, J=16.9, 10.2, 6.5, 6.5 Hz, 1H), 5.44 (dd, J=8.7, 4.9 Hz, 1H), 5.10 (s, 1H), 5.02 (s, 2H), 5.01-4.88 (m, 4H), 4.25 (dd, J=7.5, 3.7 Hz, 1H), 3.73 (dd, J=6.3, 1.5 Hz, 2H), 3.05-2.99 (m, 1H), 2.71 (d, J=7.5 Hz, 1H), 2.16-1.95 (m, 3H), 1.80 (s, 3H), 1.69 (ddd, J=13.9, 9.5, 5.9 Hz, 1H), 1.61-1.48 (m, 2H), 1.41 (d, J=1.9 Hz, 3H). ¹³C NMR (126 MHz, CDCl₃) δ 169.9, 167.0, 142.8, 142.7, 138.5, 138.2, 137.9, 133.3, 132.0, 131.1, 129.8, 127.1, 124.1, 116.6, 115.9, 115.3, 81.7, 75.6, 64.3, 61.9, 40.5, 38.9, 38.6, 38.2, 29.8, 18.9, 18.2.

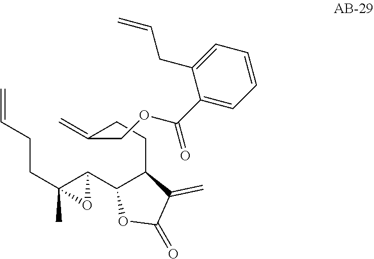 [SMILES: C=CCC[C@@]1(C)O[C@@H]1[C@H]1OC(=O)C(=C)[C@@H]1CCC(=C)COC(=O)c1ccccc1CC=C]

AB-29: To a solution of AB-28 (5 mg, 0.017 mmol) in dichloromethane (3 ml, dry) 2-allyl benzoic acid (5 eq., 13 mg, 0.085 mmol), N,N′-Dicyclohexylcarbodiimide (2 eq., 7 mg, 0.35 mmol), and catalytic DMAP were added while stirring. Reaction was stirred at room temperature for 16 hours, solvent was removed in vacuo and the crude reaction resuspended in carbon tetrachloride, then filtered to remove the DCU by-product. The crude material was then purified by flash chromatography on silica (eluting mixture 15% EtOAc in hexanes) to afford AB-29 (6.0 mg, 81%) as a solid. ¹H NMR (400 MHz, CDCl₃) δ 7.86 (d, J=7.7 Hz, 1H), 7.54-7.33 (m, 1H), 7.32-7.20 (m, 3H), 6.32 (d, J=2.4 Hz, 1H), 5.97 (ddt, J=16.7, 10.2, 6.4 Hz, 1H), 5.73 (dddd, J=16.9, 10.3, 6.6, 6.6 Hz, 1H), 5.64 (d, J=2.1 Hz, 1H), 5.18 (s, 1H), 5.05-4.92 (m, 5H), 4.73 (s, 2H), 4.11 (dd, J=8.0, 3.7 Hz, 1H), 3.74 (d, J=6.4 Hz, 2H), 2.93 (d, J=9.0 Hz, 1H), 2.71 (d, J=7.9 Hz, 1H), 2.23-1.98 (m, 4H), 1.96-1.41 (m, 5H), 1.33 (s, 3H), 1.21-1.01 (m, 1H). ¹³C NMR (100 MHz, CDCl₃) δ 167.6, 165.1, 140.6, 140.0, 135.6, 135.5, 135.4, 130.5, 129.3, 128.7, 127.4, 124.4, 121.5, 113.9, 113.6, 112.0, 79.7, 64.99, 62.0, 58.7, 40.1, 36.5, 35.6, 29.5, 27.5, 27.4, 15.6.

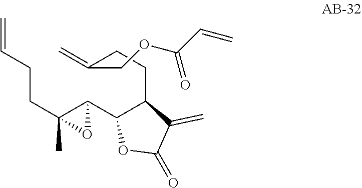 [SMILES: C=CCC[C@@]1(C)O[C@@H]1[C@H]1OC(=O)C(=C)[C@@H]1CCC(=C)COC(=O)C=C]

AB-32: To a solution of AB-28 (10 mg, 0.034 mmol) in dimethyl formamide (2 mL, dry) allyl iodide (25 eq., 80 μL, 0.85 mmol) and cesium carbonate (10 eq., 110 mg, 0.34 mmol) were charged and stirred at room temperature for 20 hrs. Reaction was diluted with DCM (10 ml), washed with water (3×10 mL) and brine, then the crude organic layer dried over sodium sulfate and concentrated in vacuo. Crude material was then purified by flash chromatography on silica (eluting mixture 15% EtOAc in hexanes) to afford AB-29 (4.1 mg, 38%) as an oil. ¹H NMR (500 MHz, CDCl₃) δ 6.34 (d, J=2.4 Hz, 1H), 5.97-5.85 (m, 1H), 5.75 (ddt, J=16.8, 10.1, 6.6 Hz, 1H), 5.67 (d, J=2.1 Hz, 1H), 5.35 (dt, J=17.3, 1.5 Hz, 1H), 5.27 (dt, J=10.4, 1.3 Hz, 1H), 5.15 (s, 1H), 5.03 (dq, J=17.2, 1.7 Hz, 1H), 5.00-4.95 (m, 2H), 4.62 (dt, J=5.9, 1.3 Hz, 2H), 4.57 (s, 2H), 4.11 (dd, J=8.0, 3.8 Hz, 1H), 2.93 (td, J=6.5, 3.5 Hz, 1H), 2.72 (d, J=7.9 Hz, 1H), 2.24-2.04 (m, 4H), 1.87-1.70 (m, 3H), 1.55-1.45 (m, 2H), 1.37 (s, 3H). ¹³C NMR (126 MHz, CDCl₃) δ 170.0, 155.5, 142.7, 138.3, 138.2, 132.2, 123.8, 119.8, 116.1, 115.2, 82.1, 70.8, 69.3, 64.6, 61.3, 42.8, 38.3, 32.1, 30.00, 18.3.

General conditions for ring-closing metathesis of di-primary alkene parthenolide derivatives AB-29, 30, 31, 32, 33, 34, 35: To a solution of the di-primary alkene parthenolide derivative in dichloromethane (10 mL, dry) Grubbs catalyst M204 (0.3 eq., Grubbs catalyst 2^{nd} generation) was added and the reaction was stirred at room temperature for 16 hours under argon. The crude reaction mixture was then transferred directly to two rounds of purification by flash chromatography on silica (eluting mixture 30% EtOAc in hexanes).

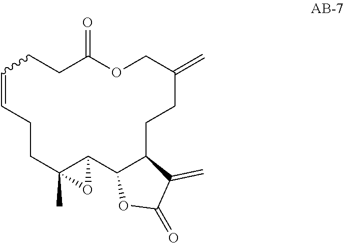 [SMILES: C=C1CC[C@H]2C(=C)C(=O)O[C@@H]2[C@H]2O[C@]2(C)CCC=CCCC(=O)OC1]

AB-7: Standard conditions were applied using AB-31 (4.6 mg, 0.012 mmol) and Grubbs catalyst M204 (0.3 eq, 3 mg, 0.0037 mmol) in 10 mL dry DCM to afford AB-7 (3.6 mg, 84%) as an oil. ¹H NMR (500 MHz, CDCl₃) δ 6.35 (d, J=2.6 Hz, 1H), 5.74 (d, J=2.3 Hz, 1H), 5.49-5.30 (m, 2H), 5.14 (s, 1H), 5.00 (s, 1H), 4.77 (d, J=12.3 Hz, 1H), 4.50 (d, J=12.4 Hz, 1H), 4.08 (dd, J=8.0, 4.3 Hz, 1H), 2.85 (1, 2H), 2.71 (d, J=8.0 Hz, 1H), 2.57-1.57 (m, 7H), 1.40 (s, 3H), 1.38-1.26 (m, 1H). ¹³C NMR (126 MHz, CDCl₃) δ 173.4, 170.2, 165.4, 143.7, 138.6, 138.5, 131.9, 131.3, 130.1, 130.0, 124.1, 124.0, 116.6, 115.9, 82.9, 82.0, 67.5, 67.0, 65.5, 64.5, 62.1, 61.4, 54.1, 43.13, 42.9, 38.7, 38.6, 34.7, 34.53, 32.5, 31.9, 31.0, 30.8, 30.4, 30.0, 28.3, 24.4, 24.0, 19.7, 17.6. HRMS (ESI) m/z calculated for C₂₀H₂₆O₅Na⁺ [M+Na]⁺: 369.1678, found: 369.1664.

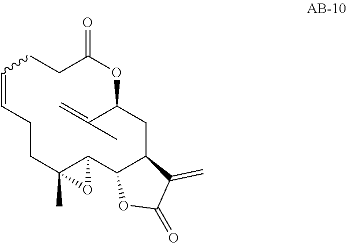 [SMILES: C=C(C)[C@@H]1C[C@H]2C(=C)C(=O)O[C@@H]2[C@H]2O[C@]2(C)CCC=CCCC(=O)O1]

AB-10: Standard condition was applied using AB-30 (5.3 mg, 0.014 mmol) and Grubbs catalyst M204 (0.3 eq, 3.6 mg, 0.0042 mmol) in 10 mL dry DCM to afford AB-7 (4.5 mg, 93%) as an oil. ¹H NMR (500 MHz, CDCl₃) δ 6.34 (d, J=1.8 Hz, 1H), 5.78 (d, J=1.6 Hz, 1H), 5.50-5.21 (m, 3H), 4.97-4.85 (m, 3H), 4.14 (d, J=9.1 Hz, 1H), 3.01-2.94 (m, 1H), 2.60 (d, J=9.1 Hz, 1H), 2.58-2.31 (m, 2H), 2.25-2.00 (m, 3H), 1.89-1.71 (m, 2H), 1.68 (s, 3H), 1.33 (s, 3H), 1.31-1.11 (m, 2H). ¹³C NMR (126 MHz, CDCl₃) δ 172.5, 169.9, 143.4, 139.1, 131.5, 130.4, 124.5, 113.4, 81.0, 74.9, 64.9, 62.0, 41.0, 40.2, 37.7, 35.6, 30.0, 29.6, 19.1. HRMS (ESI) m/z calculated for C₂₀H₂₆O₅Na⁺ [M+Na]⁺: 369.1678, found: 369.1669.

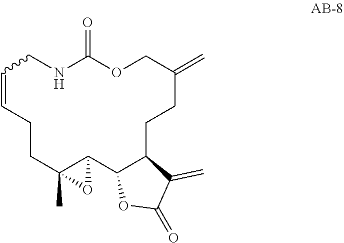 [SMILES: C=C1CC[C@H]2C(=C)C(=O)O[C@@H]2[C@H]2O[C@]2(C)CCC=CCNC(=O)OC1]

AB-8: Standard conditions were applied using AB-35 (6.8 mg, 0.018 mmol) and Grubbs catalyst M204 (0.3 eq, 4.6 mg, 0.005 mmol) in 10 mL dry DCM to afford AB-7 (2.7 mg, 0.008 mmol, 43% isolated yield) as an oil. ¹H NMR (500 MHz, CDCl₃) δ 6.32 (d, J=2.6 Hz, 1H), 5.69 (d, J=2.3 Hz, 1H), 5.55 (ddd, J=14.8, 6.4, 6.4 Hz, 1H), 5.49 (ddd, J=15.2, 4.5, 4.5 Hz, 1H), 5.06 (s, 1H), 4.96 (s, 1H), 4.95-4.91 (m, 1H), 4.88 (d, J=12.6 Hz, 1H), 4.48 (d, J=12.6 Hz, 1H), 4.31 (dd, J=4.6, 4.6 Hz, 1H), 3.82 (ddd, J=12.7, 5.7, 5.7 Hz, 1H), 3.66 (ddd, J=16.1, 4.1, 4.1 Hz, 1H), 2.93 (s, 1H), 2.69 (d, J=5.0 Hz, 1H), 2.29-2.12 (m, 2H), 2.12-1.99 (m, 1H), 1.91-1.82 (m, 1H), 1.80-1.71 (m, 1H), 1.61-1.53 (m, 1H), 1.46 (s, 3H), 1.33-1.20 (m, 4H). ¹³C NMR (126 MHz, CDCl₃) δ 170.3, 157.1, 145.0, 138.5, 130.8, 128.8, 123.8, 116.0, 80.5, 67.3, 64.9, 62.3, 43.3, 42.9, 39.4, 33.3, 30.2, 29.6, 18.6. HRMS (ESI) m/z calculated for C₁₉H₂₅NO₅Na⁺ [M+Na]⁺: 370.1631, found: 370.1626.

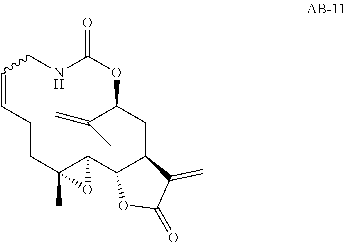 [SMILES: C=C(C)[C@@H]1C[C@H]2C(=C)C(=O)O[C@@H]2[C@H]2O[C@]2(C)CCC=CCNC(=O)O1]

AS-11: Standard conditions were applied using AB-34 (5.3 mg, 0.014 mmol) and Grubbs catalyst M204 (0.3 eq, 3.6 mg, 0.0042 mmol) in 10 mL dry DCM to afford AB-7 (3.5 mg, 71%) as an oil. ¹H NMR (500 MHz, CDCl₃) δ 6.29 (d, J=2.4 Hz, 1H), 5.62 (d, J=2.1 Hz, 1H), 5.58 (dd, J=14.9, 7.5 Hz, 1H), 5.47 (ddd, J=15.4, 5.4, 5.4 Hz, 1H), 5.27 (dd, J=6.6, 1.3 Hz, 1H), 5.00 (s, 1H), 4.94 (s, 1H), 4.90 (d, J=6.0 Hz, OH), 4.48-4.44 (m, 1H), 3.80-3.66 (m, 1H), 3.68-3.49 (m, 2H), 3.15-3.07 (m, 1H), 2.89 (d, J=3.6 Hz, 1H), 2.22-2.15 (m, 2H), 2.09-2.00 (m, 2H), 1.94 (d, J=9.9 Hz, 1H), 1.75 (s, 3H), 1.32-1.19 (m, 4H), 0.90-0.72 (m, 1H). ¹³C NMR (126 MHz, CDCl₃) δ 170.0, 143.5, 138.7, 123.7, 112.9, 112.2, 63.9, 43.6, 30.4, 19.8. HRMS (ESI) m/z calculated for C₁H₂₅NO₅Na⁺ [M+Na]⁺: 370.1631, found: 370.1627.

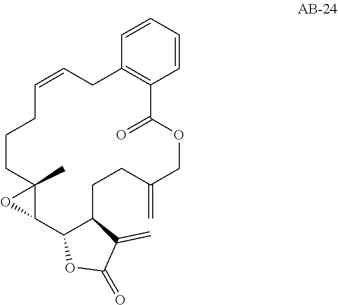 [SMILES: C=C1CC[C@H]2C(=C)C(=O)O[C@@H]2[C@@H]2O[C@]2(C)CCC/C=C\Cc2ccccc2C(=O)OC1]

AB-24: Standard conditions were applied using AB-29 (6.0 mg, 0.014 mmol) and Grubbs catalyst M204 (0.3 eq, 3.6 mg, 0.0042 mmol) in 10 mL dry DCM to afford AB-7 (3.3 mg, 57%) as an oil. ¹H NMR (500 MHz, CDCl₃) δ 7.86 (dd, J=7.7, 1.5 Hz, 1H), 7.43 (ddd, J=7.5, 7.5, 1.5 Hz, 1H), 7.28 (ddd, J=7.6, 7.6, 1.3 Hz, 1H), 7.22 (dd, J=7.7, 1.2 Hz, 1H), 6.34 (d, J=2.6 Hz, 1H), 5.74 (d, J=2.3 Hz, 1H), 5.55 (ddd, J=15.1, 7.5, 5.5 Hz, 1H), 5.40 (ddd, J=15.2, 7.1, 7.1 Hz, 1H), 5.21 (s, 1H), 5.12-5.03 (m, 1H), 4.90 (d, J=12.5 Hz, 1H), 4.77 (d, J=12.5 Hz, 1H), 4.09 (dd, J=8.1, 4.2 Hz, 1H), 3.92 (dd, J=14.4, 5.4 Hz, 1H), 3.46 (dd, J=14.3, 7.5 Hz, 1H), 2.90 (ddddd, J=7.0, 7.0, 4.5, 2.4, 2.4 Hz, 1H), 2.69 (d, J=8.1 Hz, 1H), 2.35-2.23 (m, 2H), 2.15 (q, J=6.8 Hz, 2H), 2.06-2.01 (m, 1H), 1.85 (dddd, J=9.5, 7.1, 7.1, 2.3 Hz, 2H), 1.34 (s, 3H), 1.31-1.16 (m, 3H). ¹³C NMR (126 MHz, CDCl₃) δ 170.2, 168.9, 143.3, 141.6, 138.7, 133.0, 132.0, 131.7, 130.8, 130.6, 127.3, 124.1, 116.7, 81.9, 68.2, 65.0, 61.7, 43.1, 38.6, 38.4, 32.4, 31.4, 30.0, 24.7, 18.9. HRMS (ESI) m/z calculated for C₂₅H₂₃O₅Na⁺ [M+Na]⁺: 431.1835, found: 431.1827.

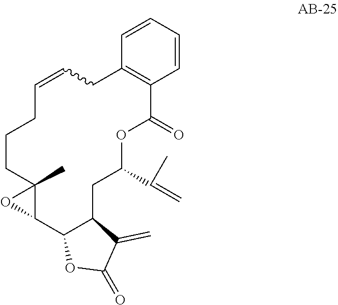 [SMILES: C=C(C)[C@@H]1C[C@H]2C(=C)C(=O)O[C@@H]2[C@@H]2O[C@]2(C)CCCC=CCc2ccccc2C(=O)O1]

AB-25: Standard conditions were applied using AB-33 (5.5 mg, 0.013 mmol) and Grubbs catalyst M204 (0.3 eq, 3.3 mg, 0.0039 mmol) in 10 mL dry DCM to afford AB-25 (3.0 mg, 64%) as an oil. ¹H NMR (500 MHz, CDCl₃) δ 7.84 (dd, J=7.8, 1.4 Hz, 1H), 7.67 (dd, J=7.6, 1.4 Hz, 1H), 7.48-7.41 (m, 1H), 7.32 (ddd, J=7.7, 7.6, 1.3 Hz, 1H), 6.31 (d, J=1.9 Hz, 1H), 5.71 (d, J=1.7 Hz, 1H), 5.34 (dd, J=10.4, 3.5 Hz, 1H), 5.28-5.14 (m, 2H), 5.09 (s, 1H), 5.03 (s, 1H), 4.48 (dd, J=15.8, 8.8 Hz, 1H), 4.41 (dd, J=6.5, 1.7 Hz, 1H), 3.95 (dd, J=16.0, 6.8 Hz, 1H), 3.66 (ddd, J=15.9, 2.9, 2.9 Hz, 1H), 3.13 (ddd, J=10.9, 4.0, 1.9 Hz, 1H), 3.01 (dd, J=15.8, 3.1 Hz, 1H), 2.65 (d, J=5.4 Hz, 1H), 2.63-2.54 (m, 1H), 2.29-1.84 (m, 5H), 1.79 (s, 3H), 1.62-1.46 (m, 3H), 1.44 (s, 3H), 1.35 (dddd, J=16.9, 11.0, 5.2, 5.2 Hz, 1H), 1.29-1.02 (m, 3H). ¹³C NMR (126 MHz, CDCl₃) δ 170.0, 167.9, 143.5, 139.2, 133.1, 132.4, 131.5, 131.0, 130.5, 129.3, 127.5, 127.2, 124.4, 113.5, 82.7, 75.5, 64.4, 62.2, 33.6, 31.5, 28.9, 30.0, 24.2, 19.2, 19.0. HRMS (ESI) m/z calculated for C₂₅H₂₈O₅Na⁺ [M+Na]⁺: 431.1835, found: 431.1821.

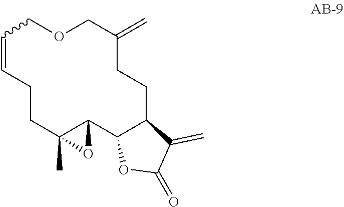 [SMILES: C=C1CC[C@H]2C(=C)C(=O)O[C@@H]2[C@@H]2O[C@]2(C)CCC=CCOC1]

AB-29: Standard conditions were applied using AB-32 (4.1 mg, 0.012 mmol) and Grubbs catalyst M204 (0.3 eq, 3.1 mg, 0.0036 mmol) in 10 mL dry DCM to afford AB-9 (3.0 mg, 80%) as an oil. ¹H NMR (500 MHz, CDCl₃) δ 6.33 (d, J=2.4 Hz, 1H), 5.79 (d, J=2.0 Hz, 1H), 5.78-5.68 (m, 1H), 5.68-5.52 (m, 2H), 5.17 (s, 1H), 5.02 (s, 1H), 4.85 (d, J=12.4 Hz, 1H), 4.68 (dd, J=12.6, 6.4 Hz, 1H), 4.56 (d, J=12.4 Hz, 1H), 4.52 (dd, J=12.7, 5.9 Hz, 1H), 4.19 (dd, J=6.4, 3.2 Hz, 1H), 3.97 (dd, J=8.8, 3.3 Hz, OH), 2.87-2.82 (m, 1H), 2.67 (d, J=6.6 Hz, 1H), 2.30-2.02 (m, 4H), 1.82 (dddd, J=14.0, 8.7, 7.2, 7.2 Hz, 1H), 1.77-1.68 (m, 1H), 1.42 (s, 3H), 1.36 (ddd, J=13.6, 9.7, 3.2 Hz, 1H). ¹³C NMR (126 MHz, CDCl₃) δ 170.3, 155.9, 143.3, 138.1, 136.5, 125.6, 124.4, 117.2, 81.4, 71.2, 68.7, 64.5, 42.8, 38.2, 33.2, 29.7, 29.3, 18.9.

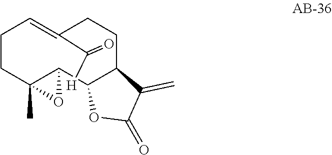 [SMILES: C=C1C(=O)O[C@@H]2[C@H]3O[C@]3(C)CC/C=C(\C=O)CC[C@@H]12]

AB-36: To a solution of 14-OH parthenolide (40 mg, 0.15 mmol) in DCM (10 mL) pyridinium chlorochromate (1.5 eq., 48 mg, 0.23 mmol) was charged. Reaction was allowed to stir for 16 hours at room temperature before confirming consumption of starting material by TLC. Reaction was quenched with saturated sodium bicarbonate, back extracted with DCM (3×), and dried over sodium sulfate. Organic layer was then concentrated in vacuo and purified by flash chromatography on silica (eluting mixture 40% EtOAc in hexanes) to afford AB-36 (31 mg, 79%) as an oil.

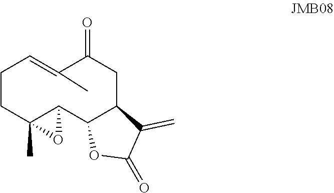 [SMILES: C=C1C(=O)O[C@@H]2[C@H]3O[C@]3(C)CC/C=C(\C)C(=O)C[C@@H]12]

JMB08: To a solution of 9(S)—OH parthenolide (62 mg, 0.23 mmol) in DCM (10 mL) pyridinium chlorochromate (1.5 eq., 75 mg, 0.35 mmol) was charged. Reaction was allowed to stir for 16 hours at room temperature before confirming consumption of starting material by TLC. Reaction was quenched with saturated sodium bicarbonate, back extracted with DCM (3×), and dried over sodium sulfate. Organic layer was then concentrated in vacuo and purified by flash chromatography on silica (eluting mixture 40% EtOAc in hexanes) to afford JMB08 (39 mg, 65%) as an oil. ¹H NMR (500 MHz, CDCl₃) δ 6.35 (d, 1H, J=3.7 Hz), 6.24 (ddq, 1H, J=8.6, 8.6, 1.2 Hz), 5.72 (d, 1H, J=3.2 Hz, 1H), 4.00 (t, 1H, J=9.0 Hz), 3.18 (dd, 1H, J=13.5, 8.5 Hz), 2.82-2.76 (m, 1H), 2.72 (dd, 1H, J=13.5, 1.6 Hz), 2.67-2.58 (m, 1H), 2.75 (dd, 1H, J=8.6, 2.0 Hz), 2.45-2.36 (m, 1H), 2.32 (ddd, 1H, J=12.7, 7.0, 1.4 Hz), 1.87 (d, 3H, J=1.4 Hz), 1.48 (s, 3H), 1.30 (ddd, 1H, J=12.1, 12.1, 7.2 Hz). ¹³C NMR (500 MHz, CDCl₃) δ 203.2, 168.7, 140.6, 138.9, 137.8, 122.3, 81.7, 66.1, 61.5, 45.2, 40.7, 36.1, 24.4, 18.6, 13.4. HRMS(ESI): m/z calculated for C₂₁H₂₂O₅S [M+Na]⁺: 285.1103; found: 285.1093.

General procedure for Wittig olefination of AB-36 and JMB08: To flame dried round-bottom flask under argon methyltriphenylphosphonium bromide (2.0 eq.) was added while warm and purged with argon for 5 minutes. To the flask dry THF (2 mL) was charged, followed by potassium tert-butoxide (1.3 eq.) as a 1 M solution in dry THF added via syringe over 1 minute, resulting in a bright yellow slurry. Reaction was allowed to stir for 1 hour at room temperature, then was cooled to −78° C. via dry ice/acetone bath. After stirring for 10 minutes at −78° C. AB-36 or JMB08 (1.0 eq.) was charged dropwise as a solution in 1 mL dry THF over 5 minutes. Reaction was allowed to gradually warm to room temperature over 1 hour. Reaction was quenched with saturated ammonium chloride, back extracted with DCM (3×), and dried over sodium sulfate. Organic layer was then concentrated in vacuo and purified by flash chromatography on silica (eluting mixture 20% EtOAc in hexanes).

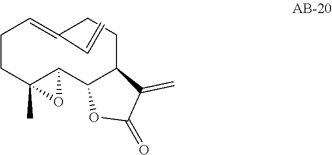 [SMILES: C=C/C1=C\CC[C@@]2(C)O[C@@H]2[C@H]2OC(=O)C(=C)[C@@H]2CC1]

AB-20: Standard conditions were applied using methyltriphenylphosphonium bromide (68 mg, 0.19 mmol), potassium tert-butoxide (130 μL of 1 M stock solution in dry THF, 0.130 mmol) and AB-36 (25 mg. 0.095 mmol) to afford AB-20 (7.2 mg, 31%) as a crystalline solid. ¹H NMR (500 MHz, CDCl₃) δ 6.73 (dd, J=17.6, 11.1 Hz, 1H), 6.32 (d, J=3.7 Hz, 1H), 5.61 (d, J=3.3 Hz, 1H), 5.45 (dd, J=12.3, 3.2 Hz, 1H), 5.25 (d, J=17.5 Hz, 1H), 5.17 (d, J=11.0 Hz, 1H), 3.78 (dd, J=8.7, 8.7 Hz, 1H), 2.91 (dd, J=13.9, 6.6 Hz, 1H), 2.81-2.74 (m, 2H), 2.66 (qd, J=12.9, 5.5 Hz, 1H), 2.27-2.15 (m, 2H), 2.07 (dd, J=13.0, 13.0 Hz, 1H), 1.77 (dddd, J=15.3, 12.1, 8.2, 1.6 Hz, 1H), 1.33-1.21 (m, 6H). ¹³C NMR (126 MHz, CDCl₃) δ 169.9, 14.0, 137.0, 132.6, 131.3, 122.0, 115.2, 83.4, 66.7, 62.1, 48.5, 37.2, 36.3, 32.4, 23.9, 18.2. HRMS (ESI) m/z calculated for C₁₆H₂₀O₃Na⁺ [M+Na]⁺: 283.1310, found: 283.1303.

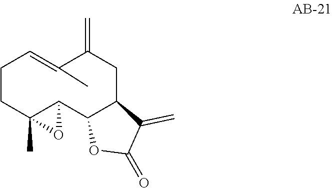 [SMILES: C=C1C[C@H]2C(=C)C(=O)O[C@@H]2[C@H]2O[C@]2(C)CC/C=C/1C]

AB-20: Standard conditions were applied using methyltriphenylphosphonium bromide (54 mg, 0.15 mmol), potassium tert-butoxide (100 μL of 1 M stock solution in dry THF, 0.0988 mmol) and JMB08 (20 mg. 0.076 mmol) to afford AB-21 (5.5 mg, 34%) as a crystalline solid. ¹H NMR (500 MHz, CDCl₃) δ 6.33 (d, J=3.7 Hz, 1H), 5.66 (d, J=3.3 Hz, 1H), 5.27 (ddd, J=11.1, 5.0, 1.5 Hz, 1H), 4.91 (d, J=2.5 Hz, 1H), 4.88 (d, J=1.8 Hz, 1H), 3.92 (dd, J=8.6, 8.6 Hz, 1H), 2.78 (ddddd, J=8.7, 8.7, 3.4, 3.4, 1.6 Hz, 1H), 2.73 (d, J=8.6 Hz, 1H), 2.67 (dd, J=14.4, 1.8 Hz, 1H), 2.42 (dd, J=14.4, 9.0 Hz, 1H), 2.39-2.24 (m, 2H), 2.18 (ddd, J=12.8, 5.5, 2.1 Hz, 1H), 1.77 (s, 3H), 1.35 (s, 3H), 1.29-1.20 (m, 3H). ¹³C NMR (126 MHz, CDCl₃) δ 170.0, 153.2, 139.3, 138.1, 128.1, 121.7, 113.0, 82.5, 66.9, 62.1, 49.5, 38.2, 37.0, 30.4, 24.7, 18.3, 17.5. HRMS (ESI) m/z calculated for C₁₆H₂₀O₃Na [M+Na]⁺: 283.1310, found: 283.1301.

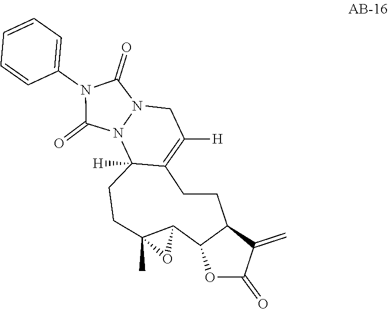 [SMILES: C=C1C(=O)O[C@@H]2[C@H]3O[C@]3(C)CC[C@@H]3C(=CCn4c(=O)n(-c5ccccc5)c(=O)n43)CC[C@@H]12]

AB-16: To a solution of AB-20 (7.2 mg, 0.028 mmol) in dry DCM (2 ml) PTAD (9.8 mg, 2 eq., 0.056 mmol) as a solid in a single portion. Reaction was allowed to stir at room temperature for 30 min before confirming consumption of starting material by TLC. Crude reaction mixture was directly purified by flash chromatography on silica (eluting mixture 60% EtOAc in hexanes) to afford AB-16 (5.1 mg, 64%) as a clear oil. ¹H NMR (500 MHz, CD₂Cl₂) δ 7.53-7.31 (m, 5H), 6.25 (d, J=3.5 Hz, 1H), 5.83-5.78 (m, 1H), 5.56 (d, J=3.1 Hz, 1H), 4.76-4.72 (m, 1H), 4.25 (dd, J=16.5, 4.2 Hz, 1H), 4.05-3.94 (m, 2H), 3.30 (d, J=9.7 Hz, 1H), 3.14 (ddddd, J=12.2, 7.2, 3.5, 3.5, 3.4 Hz, 1H), 2.49 (dd, J=13.0, 5.1 Hz, 1H), 2.23-2.13 (m, 3H), 2.11-2.01 (m, 1H), 1.96-1.81 (m, 2H), 1.74 (ddd, J=13.4, 13.3, 3.3 Hz, 1H), 1.50 (s, 3H). ¹³C NMR (126 MHz, CD₂Cl₂) δ 169.8, 155.7, 153.9, 152.7, 139.7, 139.2, 137.5, 132.1, 129.8, 129.8, 129.1, 128.9, 126.5, 126.3, 126.1, 121.9, 119.8, 81.4, 66.3, 61.2, 55.2, 44.6, 43.8, 37.4, 32.6, 30.4, 29.1, 18.9. HRMS (ESI) m/z calculated for C₂₅H₂₅N₃O₅⁺ [M+H]⁺: 436.1867, found: 436.1867.

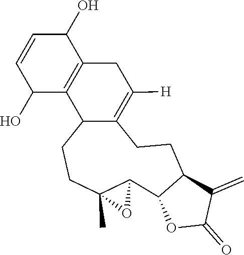 [SMILES: C=C1C(=O)O[C@@H]2[C@H]3O[C@]3(C)CCC3C(=CCC4=C3C(O)C=CC4O)CC[C@@H]12]

AB-19: To a solution of AB-20 (9.0 mg, 0.035 mmol) in dry toluene (4 ml) benzoquinone (19 mg, 5 eq., 0.175 mmol) was charged, and the resulting mixture was heated to 80° C. and stirred for 48 hours. The solvent was then removed in vacuo and the crude reaction mixture was purified by flash chromatography on silica to afford AB-19 (7.4 mg, 58%) as a solid. ¹H NMR (500 MHz, MeOD) δ 6.45 (d, J=8.5 Hz, 1H), 6.41 (d, J=8.5 Hz, 1H), 6.11 (d, J=3.4 Hz, 1H), 5.73 (dd, J=5.2, 2.4 Hz, 1H), 5.55 (d, J=3.0 Hz, 1H), 4.16 (dd, J=9.2, 9.2 Hz, 1H), 3.87-3.82 (m, 1H), 3.54 (d, J=9.7 Hz, 1H), 3.33-3.24 (m, 2H), 3.20 (dddd, J=8.4, 8.4, 3.5, 3.5 Hz, 1H), 3.07 (d, J=22.1 Hz, 1H), 2.36-2.30 (m, 2H), 2.15-2.06 (m, 1H), 2.05-1.89 (m, 2H), 1.87-1.74 (m, 2H), 1.74-1.64 (m, 1H), 1.49 (s, 3H). ¹³C NMR (126 MHz, MeOD) δ 171.2, 147.7, 147.4, 141.3, 140.3, 130.3, 122.8, 120.6, 112.7, 112.1, 82.4, 66.8, 62.2, 54.1, 44.6, 37.4, 36.5, 35.1, 31.7, 25.6, 20.7, 18.4. HRMS (ESI) m/z calculated for C₂₂H₂₇O₅⁺ [M+H]⁺: 369.1697, found: 369.1689.

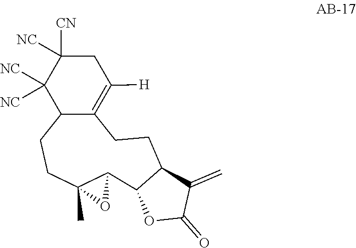 [SMILES: C=C1C(=O)O[C@@H]2[C@H]3O[C@]3(C)CCC3C(=CCC(C#N)(C#N)C3(C#N)C#N)CC[C@@H]12]

AB-17: To a solution of AB-20 (12.0 mg, 0.046 mmol) in dry DCM (2 ml) tetracyanoethylene (17.7 mg, 3 eq., 0.138 mmol) as a solid in a single portion. Reaction was allowed to stir at room temperature for 16 hours before confirming consumption of starting material by TLC. Crude reaction mixture was directly purified by flash chromatography on silica (eluting mixture 30% EtOAc in hexanes) to afford AB-17 (11.7 mg, 69%) as a clear oil. ¹H NMR (500 MHz, CDCl₃) δ 6.28 (d, J=3.4 Hz, 1H), 5.83 (dd, J=5.4, 1.8 Hz, 1H), 5.53 (d, J=3.1 Hz, 1H), 3.87 (dd, J=9.3, 9.3 Hz, 1H), 3.29-3.19 (m, 2H), 3.19-3.10 (m, 1H), 3.05 (d, J=9.5 Hz, 1H), 2.75 (ddddd, J=12.1, 9.3, 3.1, 3.1, 3.1 Hz, 1H), 2.59-2.52 (m, 1H), 2.49 (ddd, J=14.1, 4.9, 2.0 Hz, 1H), 2.25 (dddd, J=16.5, 5.2, 2.8, 2.8 Hz, 1H), 2.15-2.00 (m, 4H), 1.91-1.81 (m, 1H), 1.54 (s, 3H), 1.45 (ddd, J=14.0, 13.9, 3.0 Hz, 1H). ¹³C NMR (126 MHz, CDCl₃) δ 169.4, 138.3, 138.0, 122.2, 120.7, 111.6, 111.5, 111.2, 110.0, 81.2, 64.9, 61.1, 59.7, 46.5, 46.0, 42.8, 39.6, 38.9, 33.7, 33.5, 28.4, 27.5, 19.1. HRMS (ESI) m/z calculated for C₂₄H₂₇N₄O₃⁺ [M+H+2MeOH]⁺: 453.2132, found: 453.2128.

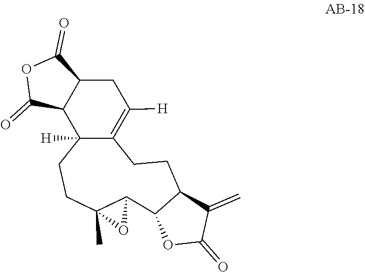 [SMILES: C=C1C(=O)O[C@@H]2[C@H]3O[C@]3(C)CC[C@@H]3C(=CC[C@@H]4C(=O)OC(=O)[C@@H]43)CC[C@@H]12]

AS-18: To a solution of AB-20 (7.6 mg, 0.029 mmol) in dry toluene (4 ml) maleic anhydride (19 mg, 3 eq., 0.175 mmol) was charged, and the resulting mixture was heated to 80° C. and stirred for 16 hours. The solvent was then removed in vacuo and the crude reaction mixture was purified by flash chromatography on silica to afford AB-18 (7.6 mg, 78%) as a solid. ¹H NMR (500 MHz, CDCl₃) δ 6.14 (d, J=3.3 Hz, 1H), 5.92-5.87 (m, 1H), 5.51 (d, J=3.2 Hz, 1H), 3.73 (dd, J=9.3, 9.3 Hz, 1H), 3.40 (q, J=8.4 Hz, 2H), 3.10-3.01 (m, 1H), 2.78 (dd, J=15.5, 7.5 Hz, 1H), 2.66 (d, J=9.5 Hz, 1H), 2.44 (dd, J=13.8, 6.5 Hz, 1H), 2.37-2.27 (m, 3H), 2.27-2.09 (m, 3H), 2.06 (dd, J=16.1, 6.4 Hz, 1H), 1.70-1.57 (m, 1H), 1.10 (dd, J=13.3, 13.3 Hz, 1H). ¹³C NMR (126 MHz, CDCl₃) δ 174.8, 173.3, 170.0, 142.4, 139.7, 123.6, 119.9, 81.5, 62.8, 60.7, 50.1, 43.7, 42.4, 42.1, 42.0, 30.4, 26.4, 26.3, 26.0, 25.3, 18.9. HRMS (ESI) m/z calculated for C₂₁H₂₆O₆⁺ [M+H+MeOH]⁺: 413.1576, found: 413.1568.

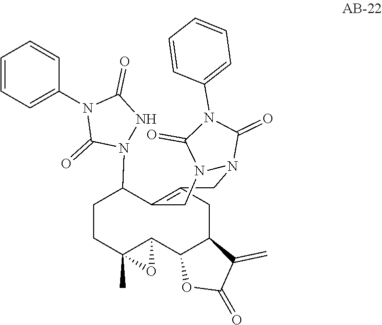 [SMILES: C=C1C(=O)O[C@@H]2[C@H]3O[C@]3(C)CCC(n3[nH]c(=O)n(-c4ccccc4)c3=O)C3=C(C[C@@H]12)Cn1c(=O)n(-c2ccccc2)c(=O)n1C3]

AB-22: To a solution of AB-21 (7.8 mg, 0.030 mmol) in dry DCM (2 ml) PTAD (10.5 mg, 2 eq., 0.06 mmol) as a solid in a single portion. Reaction was allowed to stir at room temperature for 30 min before confirming consumption of starting material by TLC. Crude reaction mixture was directly purified by flash chromatography on silica (eluting mixture 100% EtOAc) to afford AB-22 (7.8 mg, 39%) as a clear oil. ¹H NMR (500 MHz, CDCl₃) δ 9.12 (s, 1H), 7.51-7.32 (m, 10H), 6.26 (d, J=3.4 Hz, 1H), 5.52 (d, J=3.0 Hz, 1H), 5.28 (dd, J=11.7, 4.4 Hz, 1H), 4.71 (d, J=16.0 Hz, 1H), 4.27 (d, J=16.4 Hz, 1H), 4.10 (q, J=7.1 Hz, 1H), 4.04 (d, J=16.2 Hz, 1H), 3.99 (d, J=16.4 Hz, OH), 3.95 (dd, J=10.1, 8.4 Hz, 1H), 3.26 (dd, J=15.5, 8.9 Hz, 1H), 2.82-2.75 (m, 1H), 2.73 (d, J=8.5 Hz, 1H), 2.30 (d, J=15.7 Hz, 1H), 2.28-2.22 (m, 1H), 2.17 (ddd, J=13.9, 5.5, 3.0 Hz, 1H), 2.02-1.96 (m, 1H), 1.49 (s, 3H), 1.12 (ddd, J=13.0, 12.5, 5.5 Hz, 1H). ¹³C NMR (126 MHz, CDCl₃) δ 169.0, 154.7, 153.7, 153.0, 152.7, 141.2, 137.8, 131.7, 131.4, 131.4, 130.1, 130.1, 129.5, 129.3, 126.3, 126.1, 125.5, 120.5, 80.7, 63.6, 61.1, 60.5, 52.6, 48.0, 46.5, 43.7, 32.7, 28.0, 27.2, 19.3. HRMS (ESI) m/z calculated for C₃₂H₃₀N₆O₇Na⁺ [M+Na]⁺: 633.2074, found: 633.2068.

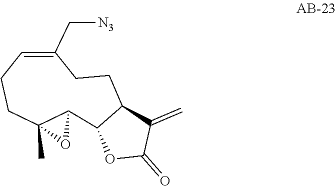 [SMILES: C=C1C(=O)O[C@@H]2[C@H]3O[C@]3(C)CC/C=C(/CN=[N+]=[N-])CC[C@@H]12]

AB-23: To a solution of 14-OH parthenolide (3.5 mg, 0.013 mmol) in dry toluene (1 ml) to which DBU (2.3 μL, 1.2 eq., 0.016 mmol) was charged while stirring under inert atmosphere. Reaction was cooled to 0-10° C. via ice bath and DPPA (4.2 μL in 100 μL dry toluene, 1.6 eq., 0.02 mmol) was added slowing over 1 minute. Reaction was stirred at 0-10° C. for 1 hr., then allowed to warm to room temperature over 16 hours. Analysis by TLC confirmed consumption of the starting material, and the crude reaction was concentrated under a stream of compressed air. Crude reaction mixture was then purified by flash chromatography on silica (eluting mixture 20% EtOAc in hexanes) to afford AB-23 (2.0 mg, 52%) as a clear oil. ¹H NMR (500 MHz, CDCl₃) δ 6.24 (d, J=3.5 Hz, 1H), 5.65 (dd, J=8.5, 8.5 Hz, 1H), 5.53 (d, J=3.2 Hz, 1H), 3.87-3.78 (m, 2H), 3.66 (d, J=13.2 Hz, 1H), 2.81 (d, J=9.4 Hz, 1H), 2.78-2.66 (m, 1H), 2.41 (dd, J=14.3, 5.5 Hz, 1H), 2.38-2.27 (m, 3H), 2.28-2.19 (m, 1H), 2.16 (ddd, J=13.1, 6.2, 1.8 Hz, 1H), 1.68 (ddd, J=14.2, 9.7, 3.9 Hz, 1H), 1.53 (s, 3H), 1.09 (ddd, J=13.3, 13.3, 2.1 Hz, 1H). ¹³C NMR (126 MHz, CDCl₃) δ 169.9, 139.3, 135.4, 131.9, 121.1, 81.6, 64.1, 60.6, 56.3, 43.5, 37.4, 26.1, 24.9, 24.5, 18.7. HRMS (ESI) m/z calculated for C₁₅H₁₉N₃O₃Na⁺ [M+Na]⁺: 312.1324, found: 312.1311.

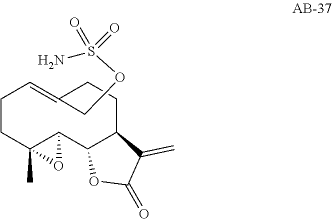 [SMILES: C=C1C(=O)O[C@@H]2[C@H]3O[C@]3(C)CC/C=C(\COS(N)(=O)=O)CC[C@@H]12]

Preparation of solid sulfamoyl chloride: To a flame dried flask under argon chlorosulfonyl isocyanate (175 μL, 2 mmol) was charged via syringe and cooled to 0-10′C via ice bath. To the flask neat formic acid (75 μL, >99%,) was charged dropwise while stirring, resulting in rapid bubbling. After two minutes the reaction had formed a solid mass to which 2 mL of dry DCM was then charged, resulting in the formation of a white suspension. Reaction was then allowed to warm to room temperature and stir for 16 hours, resulting in formation of a fine, white suspension. The crude reaction was then concentrated in vacuo to a pinkish oil, to which 5 mL dry DCM was charged to resuspend the oil and the material was again concentrated in vacuo. Redissolution/concentration was repeated an addition two times to afford sulfamoyl chloride as a crystalline off-white/pink solid which was then used directly in the following reaction.

AB-37: To a flame dried flask sulfamoyl chloride (47 mg, 6 eq., 0.4 mmol) was added and suspended in dry DCM (2 mL) to form a slurry, and the mixture was cooled 0-10° C. via ice bath. To the reaction a pre-made mixture of 14-OH parthenolide (18 mg, 0.068 mmol) and TEA (29 μL, 3 eq., 0.2 mmol) in 0.5 mL dry DCM was added dropwise over 2 minutes via syringe/needle. Reaction was stirred at 0-10′C for 30 minutes, then allowed to warm to room temperature before sampling for TLC to confirm consumption of starting material. Reaction was quenched with water, back extracted with DCM (3×), and dried over sodium sulfate. Organic layers were then concentrated in vacuo and purified by flash chromatography on silica (eluting mixture 5% MeOH in DCM) to afford AB-37 (15.6 mg, 65%) as an oil. ¹H NMR (500 MHz, CD₂Cl₂ w/MeOD additive) δ 6.28 (d, J=3.7 Hz, 1H), 5.65 (d, J=3.3 Hz, 1H), 5.59 (dd, J=12.6, 4.1 Hz, 1H), 4.81 (d, J=10.7 Hz, 1H), 4.65 (d, J=10.8 Hz, 1H), 3.86 (dd, J=8.6, 8.6 Hz, 1H), 3.00 (s, 2H), 2.83 (ddd, J=8.9, 8.3, 4.2 Hz, 1H), 2.79 (d, J=9.0 Hz, 1H), 2.68 (dd, J=14.0, 5.9 Hz, 1H), 2.48 (dddd, J=13.0, 12.9, 5.4 Hz, 1H), 2.30 (dd, J=12.4, 6.3 Hz, 1H), 2.17 (dddd, J=12.4, 8.9, 4.7, 4.7 Hz, 3H), 1.83-1.75 (m, 1H), 1.33-1.25 (m, 1H), 1.23 (s, 3H). ¹³C NMR (126 MHz, CD₂Cl₂ w/MeOD additive) δ 170.3, 139.8, 134.1, 132.9, 122.1, 83.1, 67.8, 66.8, 47.8, 43.2, 36.9, 36.7, 31.9, 24.7, 17.3.

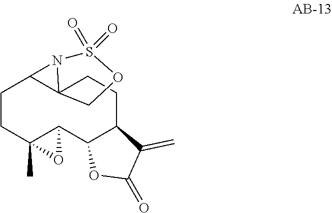 [SMILES: C=C1C(=O)O[C@@H]2[C@H]3O[C@]3(C)CCC3N4C3(CC[C@@H]12)COS4(=O)=O]

AB-13: To a solution of AB-37 (5 mg, 0.015 mmol) in 2 mL dry DCM MgO (1.5 mg, 2.5 eq., 0.036 mmol), (diacetoxyiodo)benzene (12 mg, 2.5 eq., 0.036 mmol) and rhodium acetate (1 mg, 0.1 eq., 0.002 mmol) were charged and stirred under argon atmosphere for 20 hours. The crude reaction mixture was passed through a small silica plug and flushed with EtOAc. The crude reaction mixture was then concentrated in vacuo and purified by flash chromatography on silica (eluting mixture 30% EtOAc in hexanes) to afford AB-3 (3.0 mg, 60%) as a clear oil. ¹H NMR (500 MHz, CD₂Cl₂) δ 6.30 (d, J=3.6 Hz, 1H), 5.63 (d, J=3.2 Hz, 1H), 4.71 (d, J=10.3 Hz, 1H), 4.48 (d, J=10.3 Hz, 1H), 3.85 (dd, J=9.2, 9.2 Hz, 1H), 2.87 (d, J=8.9 Hz, 1H), 2.81-2.78 (m, 1H), 2.76 (d, J=10.6 Hz, 1H), 2.70 (ddddd, J=5.8, 5.8, 2.9, 2.9, 2.8 Hz, 1H), 2.59 (ddd, J=15.4, 4.1, 4.1 Hz, 1H), 2.43 (ddd, J=13.7, 3.8, 3.8 Hz, 1H), 2.25 (dd, J=16.3, 8.9 Hz, 1H), 1.96-1.83 (m, 1H), 1.71 (dd, J=15.2, 11.4 Hz, 1H), 1.44 (s, 3H), 1.42-1.32 (m, 2H). ¹³C NMR (126 MHz, CD₂Cl₂) δ 169.0, 138.9, 121.7, 81.8, 70.5, 64.2, 60.2, 56.5, 51.6, 48.7, 36.8, 36.1, 23.6, 20.4, 18.1. HRMS (ESI) m/z calculated for C₁₅H₁₉NO₆SNa⁺ [M+Na]⁺: 364.0831, found: 364.0822.

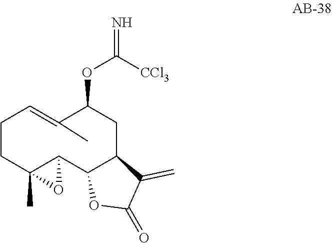

AB-38: To a solution of 9(S)—OH parthenolide (34 mg, 0.13 mmol) in dry DCM (3.4 m) DBU (9.7 μL, 0.5 eq., 0.065 mmol) and trichloroacetonitrile (195 μL, 15 eq., 1.95 mmol) were added. Reaction was stirred for 16 hours before quenching with saturated ammonium chloride, back extracting with DCM (3×), and the combined organic were dried with sodium sulfate. Organic layers were then concentrated in vacuo and purified by flash chromatography on silica (eluting mixture 15% EtOAc in hexanes) to afford AB-38 (26.0 mg, 50%) as an oil. ¹H NMR (500 MHz, CDCl₃) δ 8.36 (s, 1H), 6.36 (d, J=3.6 Hz, 1H), 5.72 (d, J=3.3 Hz, 1H), 5.53 (dd, J=11.9, 2.0 Hz, 1H), 5.31 (dd, J=10.9, 2.1 Hz, 1H), 3.86 (dd, J=8.7, 8.7 Hz, 1H), 2.96-2.89 (m, 1H), 2.70 (d, J=8.9 Hz, 1H), 2.50 (ddd, J=13.1, 13.0, 5.2 Hz, 1H), 2.38 (ddd, J=14.6, 1.9, 1.9 Hz, 1H), 2.31-2.24 (m, 1H), 2.17 (ddd, J=13.0, 5.2, 2.1 Hz, 1H), 2.12-2.04 (m, 1H), 1.80 (s, 3H), 1.32 (s, 3H), 1.29-1.21 (m, 2H). ¹³C NMR (126 MHz, CDCl₃) δ 169.4, 162.2, 138.7, 133.7, 128.7, 122.9, 92.2, 85.9, 82.5, 66.7, 62.0, 44.5, 36.7, 35.9, 24.5, 18.0, 12.4.

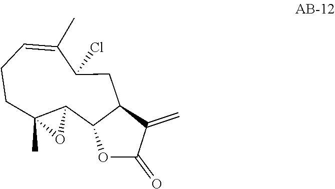 [SMILES: C=C1C(=O)O[C@@H]2[C@H]3O[C@]3(C)CC/C=C(/C)[C@@H](Cl)C[C@@H]12]

AB-12: To a solution of AB-38 (24 mg, 0.059 mmol) in dry toluene (24 ml) Bis(benzonitrile)palladium(II) chloride (1.0 eq., 0.059 mmol) was added as a solid in a single portion. Reaction was purged with argon and then stirred at 70° C. for 18 hours under inert atmosphere. The crude reaction was then concentrated in vacuo, resuspended in DCM and filtered. The crude residue was then purified by flash chromatography on silica (eluting mixture 10% EtOAc in hexanes) to afford AB-12 (10.5 mg, 63%) as a solid. ¹H NMR (500 MHz, CDCl₃) δ 6.28 (d, J=3.5 Hz, 1H), 5.58 (d, J=3.2 Hz, 1H), 5.49 (ddd, J=10.4, 9.0, 1.5 Hz, 1H), 4.73 (dd, J=12.1, 5.4 Hz, 1H), 3.85 (t, J=9.4 Hz, 1H), 2.76 (d, J=9.5 Hz, 1H), 2.69 (ddddd, J=12.5, 9.6, 3.3, 3.3, 3.3 Hz, 1H), 2.58 (ddd, J=14.1, 12.1, 3.3 Hz, 1H), 2.37-2.19 (m, 3H), 2.20-2.09 (m, 2H), 1.82-1.78 (m, 3H), 1.53 (s, 3H). ¹³C NMR (126 MHz, CDCl₃) δ 169.4, 164.1, 138.1, 136.3, 131.1, 121.4, 81.12, 63.9, 60.4, 55.4, 43.3, 37.1, 36.9, 30.4, 24.7, 18.6, 17.2. HRMS (ESI) m/z calculated for C₁₇H₁₉ClO₃⁺ [M+H]⁺: 283.1096, found: 283.1092.

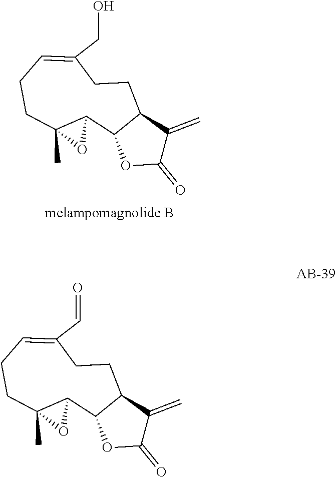 [SMILES: C=C1C(=O)O[C@@H]2[C@H]3O[C@]3(C)CC/C=C(/CO)CC[C@@H]12 ; C=C1C(=O)O[C@@H]2[C@H]3O[C@]3(C)CC/C=C(/C=O)CC[C@@H]12]

AB-39: Starting from 14-OH parthenolide, melampomagnolide B was obtained via photoisomerization in a UV chamber. This compound can be also obtained via oxidation of parthenolide (Nasim & Crooks (2008). Bioorg Med Chem Lett 18(14): 3870-3873. To a solution of melampomagnolide B (65 mg, 0.25 mmol) in DCM (10 ml) pyridinium chlorochromate (1.5 eq., 80 mg, 0.37 mmol) was charged. Reaction was allowed to stir for 16 hours at room temperature before confirming consumption of starting material by TLC. Reaction was quenched with saturated sodium bicarbonate, back extracted with DCM (3×), and dried over sodium sulfate. Organic layer was then concentrated in vacuo and purified by flash chromatography on silica (eluting mixture 40% EtOAc in hexanes) to afford AB-39 (50 mg, 77%) as an oil.

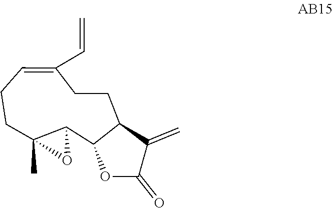 [SMILES: C=C/C1=C/CC[C@@]2(C)O[C@@H]2[C@H]2OC(=O)C(=C)[C@@H]2CC1]

AS-15: Starting from AB-39 (37 mg. 0.14 mmol), standard olefination conditions were applied using methyltriphenylphosphonium bromide (65 mg, 0.19 mmol), potassium tert-butoxide (190 μL of 1 M stock solution in dry THF, 0.190 mmol) to afford AB-15 (17 mg, 46%) as a crystalline solid.

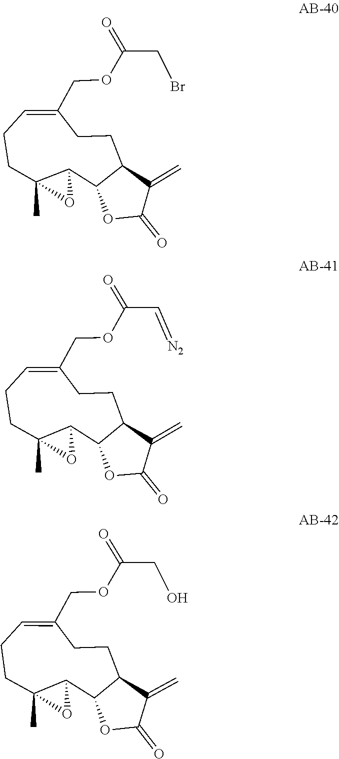

Preparation of AB-42. To a solution of melampomagnolide B (80 mg, 0.30 mmol) in dry DCM (10 ml) potassium carbonate (125 mg, 3 eq., 0.91 mmol) was charged and the reaction was cooled to 0-10° C. while stirring in an ice bath. To the reaction bromoacetyl bromide (80 μL, 3 eq., 0.91 mmol). Reaction was stirred at 0-10° C. for 2 hours before confirming consumption of starting material by TLC, then the crude reaction mixture was purified by flash chromatography on silica (eluting mixture 30% EtOAc in hexanes) to afford AB-40 (105 mg, 90%) as an oil. Material was used for subsequent diazo transfer reaction without further characterization.

To a solution of AB-40 (105 mg, 0.27 mmol) in dry THF (5 ml) N,N′-bis(p-toluenesulfonyl)hydrazine (140 mg, 1.5 eq., 0.405 mmol) was charged followed by DBU (120 μL, 3 eq., 0.81 mmol). Reaction was stirred for 45 minutes at room temperature before confirming consumption of starting material by TLC. Reaction was quenched with saturated sodium bicarbonate, back extracted with diethyl ether (3×), and dried over sodium sulfate. The crude organic layer was then filtered and dry loaded onto silica gel purified by flash chromatography on silica (eluting mixture 40% EtOAc in hexanes), and the pure fraction was dried over molecule sieves before transferring to a flame dried round-bottom flask under argon, using 10 mL dry DCM to facilitate the transfer and afford AB-41 in 10 ml DCM, 35 mL hexanes, and 15 ml EtOAc. Material was transferred to the next step without characterization.

To the solution of AB-41 rhodium acetate (18 mg, 0.15 eq., 0.04 mmol) was charged and the reaction was stirred vigorously at room temperature for 16 hours under argon. After 16 hours the crude material was purified directly via flash chromatography (eluting mixture 50% EtOAc in hexanes) to afford AB-42 (11.3 mg, 13%) as an oil.

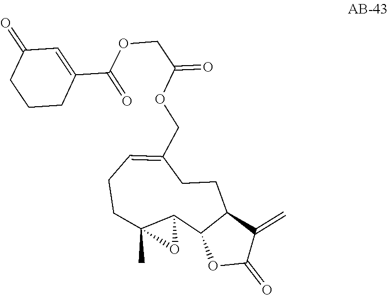 [SMILES: C=C1C(=O)O[C@@H]2[C@H]3O[C@]3(C)CC/C=C(/COC(=O)COC(=O)C3=CC(=O)CCC3)CC[C@@H]12]

AB-43: To a solution of AB-43 (11.3 mg, 0.025 mmol) in dry DCM (5 ml) 3-oxocyclohexene-1-carboxylic acid (14 mg, 4 eq., 0.1 mmol), DCC (8 mg, 1.5 eq., 0.0375 mmol), and a catalytic amount of DMAp were charged. Reaction was stirred at room temperature for 18 hours before sampling for TLC to confirm consumption of starting material. Crude material concentrated to a residue in vacuo, resuspend in carbon tetrachloride, and filtered to remove DCU precipitate. The crude material was then concentrated to a residue and purified by flash chromatography (eluting mixture 30% in hexanes) to afford AB-43 (10.2 mg, 66%) as an oil.

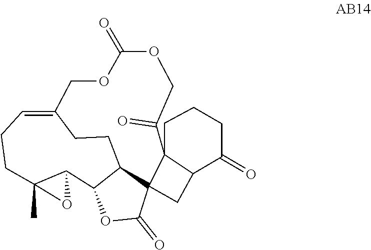

AB-14: To a flame dried, 10 mL glass flask containing solution of AB-43 (11.3 mg, 0.023 mmol) in dry DCM (8 ml) argon was spared with 20 minutes while stirring, then partially covered with aluminum foil. The reaction flask was then irradiated with light @365 nm using an Evoluchem™ LED system (p205-18-1) while stirring for 16 hours. Reaction was sampled by TLC to confirm consumption of starting material, then purified via flash chromatography (eluting mixture 30% EtOAc in hexanes) to afford AB-14 (7.0 mg, 62%).

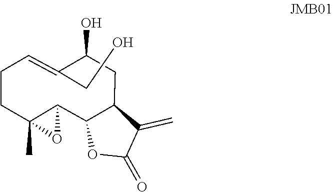 [SMILES: C=C1C(=O)O[C@@H]2[C@H]3O[C@]3(C)CC/C=C(\CO)[C@@H](O)C[C@@H]12]

JMB01: To a solution of 9-OH parthenolide (100 mg, 0.38 mmol) in DCM (20 ml, dry) selenium dioxide (42.0 mg, 1.0 eq., 0.38 mmol) was added as a solid in one portion along with tert-butyl hydroperoxide (206 ml, 3.0 eq, 0.76 mmol in 5.5 M decane) in one portion. Reaction was allowed to stir at room temperature for 36 hours while monitoring by TLC. Upon consumption of the starting material, reaction mixture was loaded onto a silica column, flushed with DCM to elute the excess peroxide, and then purified by flash chromatography (80% EtOAc in hexanes) to afford JMB01 (77.4 mg, 73%) as a foamy oil. ¹H NMR (500 MHz, CDCl₃): δ=6.34 (d, 1H, J=3.1 Hz), 5.69 (d, 1H, J=3.1 Hz), 5.51 (dd, 1H, J=12.4, 3.8 Hz), 4.51 (d, 1H, J=11.8 Hz), 4.38 (dd, 1H, J=10.8, 1.9), 4.33 (d, 1H, J=11.8 Hz), 3.82 (dd, 1H, J=8.7, 8.7 Hz), 2.87-2.81 (m, 1H), 2.66 (d, 1H, 9.0 Hz), 2.50 (ddd, 1H, J=13.5, 5.6, 5.3 Hz), 2.39 (bs, 2H), 2.30 (d, 1H, J=14.4 Hz), 2.21-2.12 (m, 2H), 1.30-1.21 (m, 1H), 1.23 (s, 3H); ¹³C NMR (126 MHz, CDCl₃) δ 169.7, 139.0, 138.8, 130.4, 122.6, 82.5, 80.7, 66.6, 61.7, 58.5, 44.6, 41.1, 36.8, 24.4, 17.7; HRMS (ESI): m/z calculated for C₁₅H₂₀O₅Na [M+Na]⁺: 303.1208; found: 303.1198.

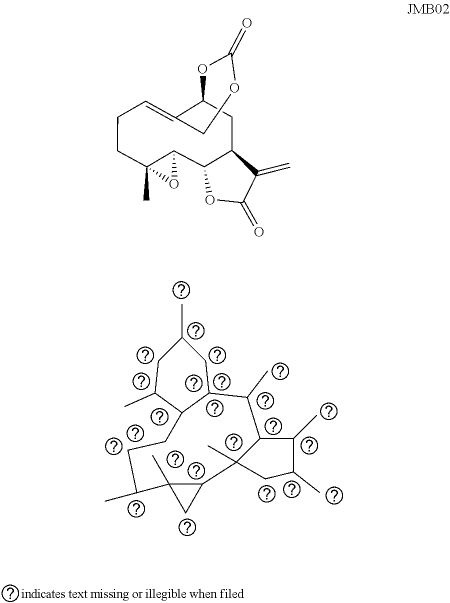

JMB02: To a solution of JMB01 (9.3 mg, 0.03 mmol) in DCM (5 ml, dry) pyridine (16 μL, 6.0 eq., 0.20 mmol) and triphosgene (3.9 mg, 0.4 eq, 0.01 mmol) dissolved in 0.5 mL dry DCM were added dropwise under inert argon atmosphere. Reaction was allowed to stir at room temperature for 16 hours while monitoring by TLC. Upon consumption of the starting material, reaction mixture was quenched with 10 mL of saturated ammonium chloride and extracted with 10 ml DCM ×3. The combined organic layer was washed with 1.0 M HCl, saturated sodium bicarbonate, and brine. The combined organic layer was dried over sodium sulfate, concentrated in vacuo, and purified by flash chromatography (90% EtOAc in hexanes) to afford JMB02 (6.4 mg, 63%) as crystalline solid. ¹H NMR (500 MHz, CDCl₃): δ=6.45 (d, 1H, J=3.2), 5.85 (dd, 1H, J=12.3, 3.8), 5.74 (d, 1H, J=2.8), 4.99 (d, 1H, J=12.6 Hz), 4.94 (dd, 1H, J=12.0, 0.6 Hz), 4.86 (d, 1H, J=12.6 Hz), 3.84 (dd, J=9.0, 9.0 Hz), 2.88-2.80 (m, 1H), 2.67 (d, 1H, 9.5 Hz), 2.62 (dd, 1H, J=14.9, 1.5 Hz), 2.52-2.44 (m, 1H), 2.39 (dddd, 1H, J=12.8, 5.6, 5.6, 5.6) 2.27 (ddd, 1H, J=13.2, 4.4, 0.6), 2.16 (ddd, 1H, J=14.9, 11.6, 8.3), 1.35 (ddd, 1H, J=12.9, 6.6, 6.3), 1.20 (s, 3H). ¹³C NMR (126 MHz, CDCl₃) δ 168.7, 150.0, 137.8, 131.5, 126.9, 123.5, 84.2, 82.5, 66.3, 64.0, 61.2, 42.4, 38.8, 36.1, 30.4, 24.9, 17.4. HRMS (ESI): m/z calculated for C₁₆H₁₃O₆Na [M+Na]⁺: 329.1001; found: 329.0991.

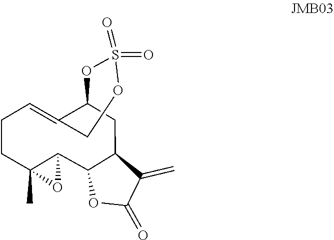

JMB03: To a solution of JMB01 (25 mg, 0.09 mmol) in DCM (25 ml, dry) triethylamine (49.7 μL, 4.0 eq, 0.36 mmol) and 4-dimethylaminopyridine (4.5 mg, 0.4 eq., 0.04 mmol) was added dropwise dissolved in 0.5 mL dry DCM under inert argon atmosphere. Reaction was cooled to −78° C. and triethylamine (50 μL, 4.0 eq, 0.36 mmol) and sulfuryl chloride (14.4 μL 2.0 eq, 0.18 mmol in a 1% stock solution in dry DCM) were added in one aliquot over 2 minutes. Reaction was allowed to stir while slowly warming to room temperature for 16 hours while monitoring by TLC. Upon consumption of the starting material, reaction mixture was quenched with 25 mL of saturated sodium bicarbonate and extracted with 25 ml DCM ×3. The combined organic layer was washed with water and washed with brine. The combined organic layer was dried over sodium sulfate, concentrated in vacuo, and purified by flash chromatography ×2 (75% EtOAc in hexanes, 10% EtOAc in DCM) to afford JMB03 (5.8 mg, 19%) as an oil. ¹H NMR (500 MHz, CDCl₃): δ=6.37 (d, 1H, J=3.3 Hz), 5.69 (d, 1H, J=3.3 Hz), 5.64 (dd, 1H, J=12.2, 4.2 Hz), 4.41 (dd, 1H, J=10.9, 2.4 Hz), 4.39 (d, 1H, J=11.3 Hz), 4.19 (d, 1H, J=11.3 Hz), 3.84 (dd, 1H, J=8.7, 8.6 Hz), 2.88-2.81 (m, 1H), 2.69 (d, 1H, J=9.0 Hz), 2.53 (dddd, 1H, J=13.8, 12.4, 12.4, 5.3 Hz), 2.45-2.37 (m, 1H), 2.30 (ddd, 1H, J=15.0, 1.8, 1.6 Hz), 2.23 (dd, 1H, J=5.1, 1.8 Hz), 2.11 (ddd, 1H, J=15.0, 10.9, 8.5 Hz), 1.32 (s, 3H). ¹³C NMR (126 MHz, CDCl₃) δ=169.4, 138.8, 137.7, 133.4, 122.6, 82.2, 79.0, 66.5, 61.5, 44.5, 40.7, 36.8, 30.4, 24.7, 18.3.

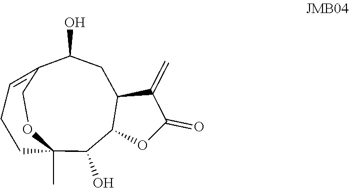 [SMILES: C=C1C(=O)O[C@H]2[C@H]1C[C@H](O)C1=CCC[C@](C)(OC1)[C@H]2O]

JMB04: To a solution of JMB01 (8.0 mg, 0.03 mmol) in DCM (5 ml, dry) tosylic acid was added. The reaction was allowed to stir at room temperature for 36 hours before confirming consumption of starting material by TLC. Reaction was quenched with saturated sodium bicarbonate, extracted with DCM (3×), and dried over sodium sulfate. Organic layer was then concentrated in vacuo and purified by flash chromatography on silica (eluting mixture 67% EtOAc in hexanes) affording JMB04 (4.4 mg, 55%) as an oil. ¹H NMR (500 MHz, CDCl₃): δ=6.32 (d, 1H, J=3.5 Hz), 5.83 (dd, 1H, J=7.1, 1.5 Hz), 5.65 (d, 1H, J=2.9 Hz), 4.55 (dd, 1H, J=7.8, 7.8 Hz), 4.37 (dd, 1H, 12.4, 0.5 Hz), 4.32 (d, 1H, J=7.5 Hz), 4.21 (dd, 1H, J=11.1, 4.9 Hz), 4.00 (dd, 1H, J=12.4, 0.6 Hz), 2.76 (dddd, 1H, J=18.0, 9.9, 1.6, 1.5 Hz), 2.62-2.55 (m, 1H), 2.41-2.23 (m, 3H), 2.22-2.15 (m, 1H), 2.09-2.04 (m, 1H), 1.70 (bs, 2H), 1.16 (s, 3H); ¹³C NMR (126 MHz, CDCl₃) δ=169.1, 141.1, 140.0, 134.0, 121.9, 81.8, 80.8, 77.9, 76.2, 58.7, 46.6, 36.7, 32.8, 26.0, 23.4. HRMS (ESI): m/z calculated for C₁₅H₂₀O₅Na [M+Na]⁺: 303.1208; found: 303.1199.

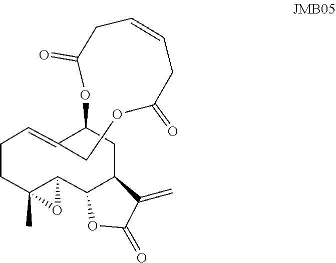 [SMILES: C=C1C(=O)O[C@@H]2[C@H]3O[C@]3(C)CC/C=C3\COC(=O)C/C=C\CC(=O)O[C@H]3C[C@@H]12]

JMB05: To a solution of JMB01 (10 mg, 0.04 mmol) in DCM (5 ml, dry) N,N′-dicyclohexylcarbodiimide (15.7 mg, 2.1 eq, 0.07 mmol in 1 mL of dry DCM), 4-dimethylaminopyridine (catalytic amount in 1 mL of dry DCM), and 3-butenoic acid (15.2 μL, 5.0 eq, 0.17 mmol) were added in an inert argon atmosphere. The reaction was allowed to stir at room temperature for 36 hours before confirming consumption of starting material by TLC. Reaction was quenched with saturated sodium bicarbonate, extracted with DCM (3×), and dried over sodium sulfate. Organic layer was then concentrated in vacuo and redissolved in chloroform, followed by filtration through a silica plug (×2). Eluate was concentrated in vacuo and redissolved in acetonitrile, followed by filtration through a silica plug (×2). Eluate was concentrated in vacuo and purified by flash chromatography on silica (eluting mixture 50% EtOAc in hexanes). To a solution of the resultant diolefin (13.6 mg, 0.03 mmol) in DCM (20 ml, dry) Grubb's 2^{nd} generation catalyst (4.2 mg, 0.15 eq, 0.005 mol) was added in an inert argon atmosphere. The reaction was allowed to stir at room temperature for 16 hours before confirming consumption of the starting material by TLC. The crude reaction mixture was concentrated in vacuo and purified by flash chromatography on silica (eluting mixture 50% EtOAc in hexanes) affording JMB05 (7.6 mg, 55% over two steps) as fine, thin clear/white crystals. ¹H NMR (500 MHz, CDCl₃): δ=6.37 (d, 1H, J=3.2 Hz), 5.92-5.83 (m, 2H), 5.83-5.76 (m, 1H), 5.65 (d, 1H, J=3.2 Hz), 5.29 (ddd, 1H, J=10.7, 1.0, 1.0 Hz), 5.08 (d, 1H, J=12.0 Hz), 4.61 (d, 1H, J=12.0 Hz), 3.79 (dd, 1H, J=8.8, 8.7 Hz), 3.19-3.03 (m, 4H), 2.91-2.84 (m, 1H), 2.65 (d, 1H, J=8.8 Hz), 2.49 (dddd, 1H, J=13.2, 13.0, 13.0, 5.1), 2.44-2.37 (m, 1H), 2.30-2.14 (m, 3H), 1.31-1.26 (m, 1H), 1.20 (s, 3H). ¹³C NMR (126 MHz, CDCl₃) δ=171.4, 170.6, 169.0, 138.2, 137.5, 131.1, 127.4, 126.0, 122.8, 81.9, 81.3, 66.2, 61.5, 58.3, 45.1, 36.4, 36.1, 34.5, 34.2, 24.7, 17.8; HRMS (ESI): m/z calculated for C₁₅H₂₀O₅Na [M+Na]⁺: 411.1420; found: 411.1411.

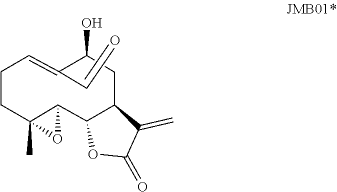 [SMILES: C=C1C(=O)O[C@@H]2[C@H]3O[C@]3(C)CC/C=C(\C=O)[C@@H](O)C[C@@H]12]

JMB01*: To a solution of JMB01 (8.0 mg, 0.03 mmol) and celite (20 mg) in DCM (8 ml, dry) pyridinium chlorochromate (18.5 mg, 3.0 eq, 0.09 mmol) was added. The reaction was allowed to stir at room temperature for 16 hours before confirming consumption of starting material by TLC. Et₂O (8 mL) was added to the reaction vessel and the crude reaction mixture was filtered through a florisil plug, eluting with Et₂O. The eluate was concentrated in vacuo and purified by flash chromatography on silica (eluting mixture 50% EtOAc in hexanes) affording JMB01* (3.0 mg, 38%) as an oil.

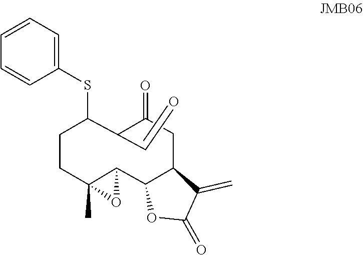 [SMILES: C=C1C(=O)O[C@@H]2[C@H]3O[C@]3(C)CCC(Sc3ccccc3)C(C=O)C(=O)C[C@@H]12]

JMB06: To a solution of JMB01* (4.0 mg, 0.01 mmol) in DCM (4 ml, dry) thiophenol (2.2 μL, 1.5 0.02 mmol in a 10% solution of dry DCM) was added in an inert argon atmosphere. The reaction was allowed to stir at room temperature for 1 hour before confirming consumption of starting material by TLC. Reaction mixture was concentrated in vacuo and purified by flash chromatography on silica (eluting mixture 25% EtOAc in hexanes) affording JMB06 (2.7 mg, 48%, d.r. 10.4:1.1:1) as an oil. Compound JMB06 was isolated as a mixture of diastereomers in a 50.0:4.5:1 ratio. ¹H NMR (500 MHz, CDCl₃) δ=9.92 (s, 1H), 7.43 (dd, 2H, J=7.4, 2.2 Hz), 7.33-7.26 (m, 3H), 6.20 (d, 1H, J=2.8 Hz), 5.38 (d, 1H, J=2.8 Hz), 4.19 (dd, 1H, J=9.1, 9.1 Hz), 3.94 (dd, 1H, J=10.0, 9.9 Hz), 3.61 (d, 1H, J=10.0 Hz), 2.79-2.60 (m, 4H), 2.50-2.40 (m, 1H), 2.18-2.08 (m, 2H), 1.94-1.86 (m, 1H), 1.24 (s, 3H). ¹³C NMR (126 MHz, CDCl₃) δ=206.7, 200.0, 168.1, 138.6, 135.9, 133.7, 130.9, 130.0, 129.9, 128.8, 121.6, 81.3, 77.7, 76.1, 55.5, 52.8, 42.9, 42.0, 38.3, 32.1, 16.7. HRMS(ESI): m/z calculated for C₂₁H₂₂O₅SNa [M+Na]⁺: 409.1086; found: 409.1072.

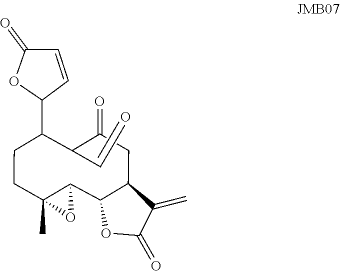 [SMILES: C=C1C(=O)O[C@@H]2[C@H]3O[C@]3(C)CCC(C3C=CC(=O)O3)C(C=O)C(=O)C[C@@H]12]

JMB07: A solution of JMB01* (4.0 mg, 0.01 mmol) in DCM (4 ml, dry) was cooled to -78° C. and 2-(Trimethylsiloxy)furan (3.7 μL, 1.5 eq, 0.02 mmol) was added in an inert argon atmosphere. The reaction was allowed to warm to room temperature while stirring for 16 hours before confirming consumption of starting material by TLC. Reaction mixture was concentrated in vacuo and purified by flash chromatography on silica (eluting mixture 25% EtOAc in hexanes) affording P23 (1.9 mg, 36%) as an oil. ¹H NMR (500 MHz, CDCl₃): δ=9.96 (s, 1H), 7.40 (dd, 1H, J=5.7, 1.6 Hz), 6.26 (d, 1H, J=3.2 Hz), 6.14 (dd, 1H, J=5.7, 2.1 Hz), 5.56 (d, 1H, J=3.0 Hz), 5.25 (ddd, 1H, J=2.1, 1.6, 1.2 Hz), 4.08 (dd, 1H, J=8.8, 8.6 Hz), 3.53 (d, 1H, J=10.0 Hz), 3.03-2.96 (m, 2H), 2.78 (dd, 1H, J=12.3, 12.2 Hz), 2.64-2.55 (m, 2H), 2.25 (ddd, 1H, J=13.8, 8.0, 8.0 Hz), 1.91 (ddd, 1H, J=13.5, 6.6, 6.6 Hz), 1.78-1.71 (m, 2H), 1.45 (s, 3H) ¹³C NMR (126 MHz, CDCl₃) δ=206.7, 200.0, 168.1, 138.6, 135.9, 133.7, 130.9, 130.0, 129.9, 128.8, 121.6, 81.3, 77.7, 76.1, 55.5, 52.8, 42.9, 42.0, 38.3, 32.1, 16.7. HRMS (ESI): m/z calculated for C₁₉H₂₀O₇Na [M+Na]⁺: 383.1107; found: 383.1084.

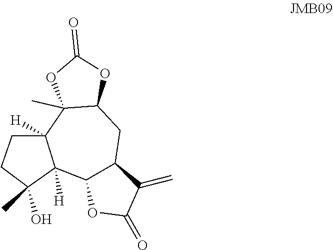 [SMILES: C=C1C(=O)O[C@@H]2[C@@H]3[C@@H](CC[C@@]3(C)O)[C@]3(C)OC(=O)O[C@H]3C[C@@H]12]

JMB09: To a solution of AB-38 in DCM (dry) tosylic acid (3.1 mg, 0.5 eq, 0.02 mmol) was added in an inert argon atmosphere. The reaction was allowed to stir until complete conversion was observed by TLC. The reaction mixture was quenched with saturated sodium bicarbonate, washed with water and brine, and dried over sodium sulfate. The organic layer was concentrated in vacuo and purified by flash chromatography on silica (eluting mixture 50% EtOAc in hexanes) to afford the product JMB09 (2.6 mg, 15% over two steps) as a crystalline solid. ¹H NMR (500 MHz, CDCl₃): δ=6.31 (d, 1H, J=3.3 Hz), 5.62 (dd, 1H, J=3.0 Hz), 4.26 (dd, 1H, J=12.4, 3.2 Hz), 4.23 (dd, 1H, J=11.9, 10.1 Hz), 3.00 (s, 1H), 2.88 (ddd, 1H, J=12.9, 10.7, 7.7 Hz), 2.72 (m, 1H), 2.61 (ddd, 1H, J=13.2, 3.2, 3.2 Hz), 2.48 (dd, 1H, J=12.4, 12.4 Hz), 2.02-1.67 (m, 5H), 1.40 (s, 3H), 1.37 (s, 3H). ¹³C NMR (500 MHz, CDCl₃) δ 168.7, 154.0, 136.9, 122.1, 88.6, 85.8, 81.8, 80.8, 54.7, 46.2, 45.2, 40.7, 25.8, 25.7, 24.3, 18.4. HRMS (ESI): m/z calc. for C₁₆H₂₀O₆Na [M+Na]⁺: 331.1158; found: 331.1151.

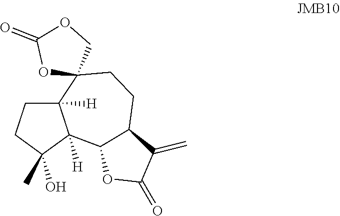

JMB10: To a solution of 14-OH parthenolide (14.0 mg, 0.05 mmol) in DCM trichloroacetyl isocyanate (2.5 eq) was added in an inert argon atmosphere. The reaction mixture was allowed to stir for 2 hours until complete conversion was observed by TLC. Alumina (50 mg/mg of hydroxyparthenolide) was added to the reaction mixture and stirred until complete consumption of the isocyanate intermediate was observed by TLC. The alumina was removed by filtering the reaction mixture through a cotton plug and washing with DCM. The crude reaction mixture was concentrated in vacuo and purified by column chromatography (eluting mixture 40% EtOAc in hexanes). To a solution of the resultant carbamate in DCM (dry) tosylic acid (2.8 mg, 0.5 eq, 0.02 mmol) was added in an inert argon atmosphere. The reaction was allowed to stir until complete conversion was observed by TLC. The reaction mixture was quenched with saturated sodium bicarbonate, washed with water and brine, and dried over sodium sulfate. The organic layer was concentrated in vacuo and purified by flash chromatography on silica (eluting mixture 50% EtOAc in hexanes) to afford the product JMB10 (2.0 mg, 12% over two steps) as a crystalline solid. ¹H NMR (500 MHz, CDCl₃): δ=6.27 (d, J=3.3 Hz, 1H), 5.56 (d, J=3.0 Hz, 1H), 4.43 (dd, J=11.5, 9.5 Hz, 1H), 4.35 (d, J=8.5 Hz, 1H), 4.00 (d, J=8.6 Hz, 1H), 2.68-2.54 (m, 3H), 2.46 (dd, J=11.9, 11.9 Hz, 1H), 2.31 (dd, J=11.4, 3.9 Hz, 1H), 2.13-2.08 (m, 1H), 1.97 (dt, J=13.8, 7.1 Hz, 1H), 1.88-1.72 (m, 4H), 1.61-1.51 (m, 1H), 1.39 (s, 3H). ¹³C NMR (126 MHz, CDCl₃) δ 169.7, 154.6, 138.6, 121.8, 86.3, 83.1, 80.2, 76.1, 56.5, 47.4, 45.5, 40.3, 39.9, 26.5, 24.4, 23.8. HRMS (ESI): m/z calc. for C₁₆H₂₀O₆Na [M+Na]⁺: 331.1158; found: 331.1150.

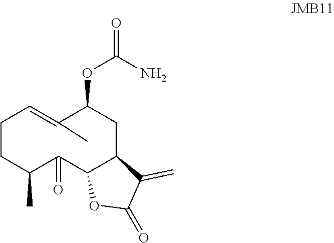 [SMILES: C=C1C(=O)O[C@@H]2C(=O)[C@@H](C)CC/C=C(\C)[C@@H](OC(N)=O)C[C@@H]12]

JMB04: To a solution of AB-38 in DCM (dry) tosylic acid (3.1 mg, 0.5 eq, 0.02 mmol) was added in an inert argon atmosphere. The reaction was allowed to stir until complete conversion was observed by TLC. The reaction mixture was quenched with saturated sodium bicarbonate, washed with water and brine, and dried over sodium sulfate. The organic layer was concentrated in vacuo and purified by flash chromatography on silica (eluting mixture 50% EtOAc in hexanes) afford the product JMB11 (3.8 mg, 22% over 2 steps) as a crystalline solid. ¹H NMR (500 MHz, CDCl₃) δ 6.34 (d, J=3.3 Hz, 1H), 5.70 (d, J=2.9 Hz, 1H), 5.05-4.99 (m, 1H), 4.66 (d, J=5.6 Hz, 1H), 4.58 (bs, 2H), 3.51 (s, 1H), 2.71-2.68 (m, 1H), 2.43-2.38 (m, 1H), 2.17 (d, J=14.0 Hz, 1H), 2.13-2.06 (m, 1H), 2.03-1.89 (m, 2H), 1.72 (s, 3H), 1.27 (d, J=7.3 Hz, 3H). ¹³C NMR (126 MHz, CDCl₃) δ 210.2, 169.4, 156.1, 138.1, 132.0, 129.6, 124.5, 80.2, 78.9, 47.1, 43.4, 37.2, 36.2, 30.1, 24.7, 14.8. HRMS (ESI): m/z calc. for C₁₆H₂₀NO₅Na [M+Na]⁺: 330.1317; found: 330.1311.

### 5.2 Example 2: Anticancer Activity of the Natural Product-Like Compounds

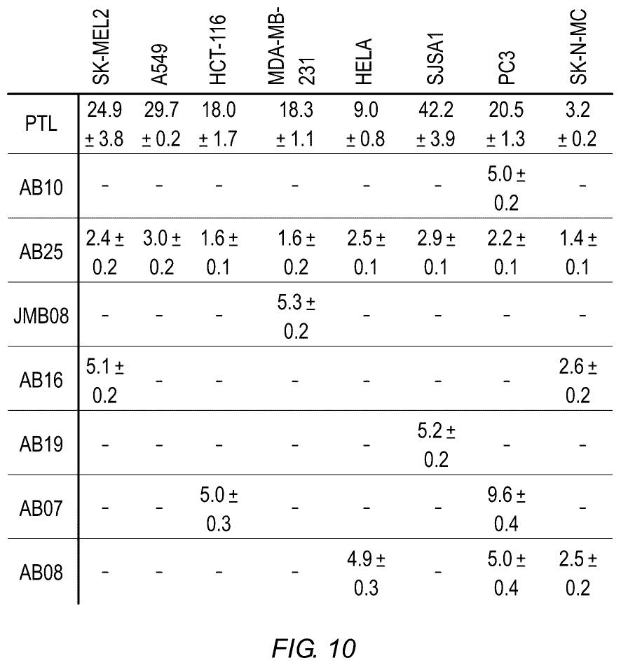

The natural product-like compounds described in the examples above have been found to possess potent activity against various types of human cancer cells, including prostate cancer (PC3), neuroblastoma (SK-N-MC), breast cancer (MDA-MB-231), lung cancer (A549), cervical cancer (HeLa), skin cancer (SK-MEL-2), osteosarcoma (SJSA-1), and colon cancer (HCT-116), while showing little to no toxicity against non-cancer cells (i.e., lung fibroblasts (WI-38)). The anticancer activity of the compounds was determined using a MTT (3-[4,5-dimethylthiazol-2-yl]-2,5 diphenyl tetrazolium bromide) assay, which measures cell viability upon incubation of the cells with the compounds. Using this assay, many of the natural product-like compounds were found to exhibit significantly (2- to >10-fold) improved anticancer activity compared to PTL, as determined based on the higher reduction in cancer cell viability upon incubation with the compound at the fixed concentrations of 10 μM and 20 μM (FIG. 9). Furthermore, several compounds exhibited more potent activity than PTL against many of the tested cancer cell lines. Moreover, the compounds were found to exhibit a diverse range of anticancer activity profiles, including compounds with broad-spectrum cytotoxicity (i.e., AB24, AB25) as well as compounds with both intermediate (e.g., AB10, JMB08, AB16, AB19) and high cancer-type specificity (e.g., JMB03, AB20, AB26). Whereas several members of the library showed significantly enhanced cytotoxicity compared to PTL and its oxyfunctionalized derivatives, most of the active compounds also showed little to no toxicity against non-cancer cells (WI-38, lung fibroblast), demonstrating that their activity is specific against cancer cells. Other notable structure-activity insights include the large impact on the linkage utilized for macrocyclization of 14-hydroxy-PTL-derived AB08 and AB24 on the cancer cell specificity of these molecules. Similarly, the cyclic sulfate vs. cyclic carbonate ring in JMB03 vs. JMB02 confers the former with selective activity against colon (HCT-116) and cervical (HeLa) cancer cells, while JMB02 displays less potent anticancer activity. Lastly, the cycloaddition products AB16 and AB19 showed potent anticancer activity against at least six cell lines, whereas the similar cycloaddition products AB17 and AB18 showed lower activity against the same cell lines, further highlighting the impact of the diversification strategies on the anticancer profile. Importantly, as determined based on their half-maximal lethal dose (LD₅₀) values (FIG. 10), for each of the cancer cell lines the compounds of the library show slightly reduced, comparable, or markedly enhanced anticancer potency compared to PTL The latter include AB25, which show 210-fold enhanced potency against lung adenocarcinoma (A549) and breast cancer cells (MDA-MB-231), and compound AB07, with >3-fold increased potency against colon cancer cells (HCT-116). Altogether, these results demonstrate the effectiveness of the strategy in delivering bioactive compounds with potent as well as diversified anticancer profiles.

The natural product-like compounds described in the examples above have been found to possess potent activity against various types of leukemias and lymphomas, including acute myelogenous leukemia (AML). The antileukemic activity of the PTL analogs was evaluated using the leukemia cell line HL-60 and the leukemia stem cell line M9-ENL1. To measure the compound activity against hematologic cells, cells were treated for 24 hours in the presence or in the absence of the PTL analog at 10 μM and 20 μM, followed by measurement of cell viability using alamarBlue stain (FIG. 9). Using this assay, several of the novel PTL analogs disclosed herein were found to exhibit significantly higher antileukemic activity compared to the parent molecule PTL. In particular, compounds such as JMB06 and JMB07 show relatively selective anticancer activity against HL-60, demonstrating the beneficial effect of scaffold diversification toward the discovery of potent antileukemic agents.

Experimental Procedures. All cancer cell lines were maintained in a 37° C. humidified incubator with 5% C02. A549, MDA-MB-231, PC-3, SK-N-MC, HeLa, SJSA-1, HCT-116, and SK-MEL-2 cells were cultured in DMEM medium (Gibco) supplemented with 10% FBS (Gibco) and 100 I.U./ml penicillin-streptomycin (Sigma). Adherent cells were dissociated using Accutase (Millipore) or trypsin-EDTA 0.25% (Gibco). Adherent cells were seeded in a 96-well plate at a density of 1000-20,000 cells per well in 180 μl complete DMEM media. Suspension cells were seeded in conical 96-well plates (Nunc) at a density of 30,000 cells per well in 90 ul of complete α-MEM media devoid of phenol red indicator. The plates were incubated in a humidified incubator for 16 hours prior to treatment. PTL analogs were prepared as concentrated stocks (2 mM) in DMSO and stored at −30° C. Prior to testing, the analogs were diluted into complete cell culture medium and the media was supplemented with sterile DMSO (10% of final volume). For the initial cytotoxicity screening, the compounds were added to the cells in 20 μl aliquots to give a final concentration of 20 μM and 10 μM parthenologs and 1% DMSO (n=3). Following a 24-hour incubation, the culture media from the adherent cell plates (e.g., HeLa, A549) was aspirated and replaced with 100 pd of 1 mg/mL Thiazolyl Blue Tetrazolium Bromide (MTT, Fisher) in complete culture medium devoid of phenol red. After incubation at 37° C. for 1 hour, the plates were centrifuged (4000 rpm, 5 minutes), the media was removed and 100 ul of DMSO was added to solubilize the formazan product. The resulting OD was measured at 550 nm using a multi-well plate reader (Tecan). Suspension cells were treated for 24 hours in the presence or in the absence of the compounds at 10 μM and 20 μM, followed by measurement of cell viability using alamarBlue stain. After incubation, 22 μM of AlamarBlue (Invitrogen) was added to each well and incubated until color change was observed, typically approximately 20-24 hours, in the control wells (1% DMSO in media, no compound). The resulting absorbance was measured at 570 nm and 600 nm using a multi-well plate reader (Tecan).

### 5.3 Example 3: Chemoinformatic Analysis of Chemical Diversity

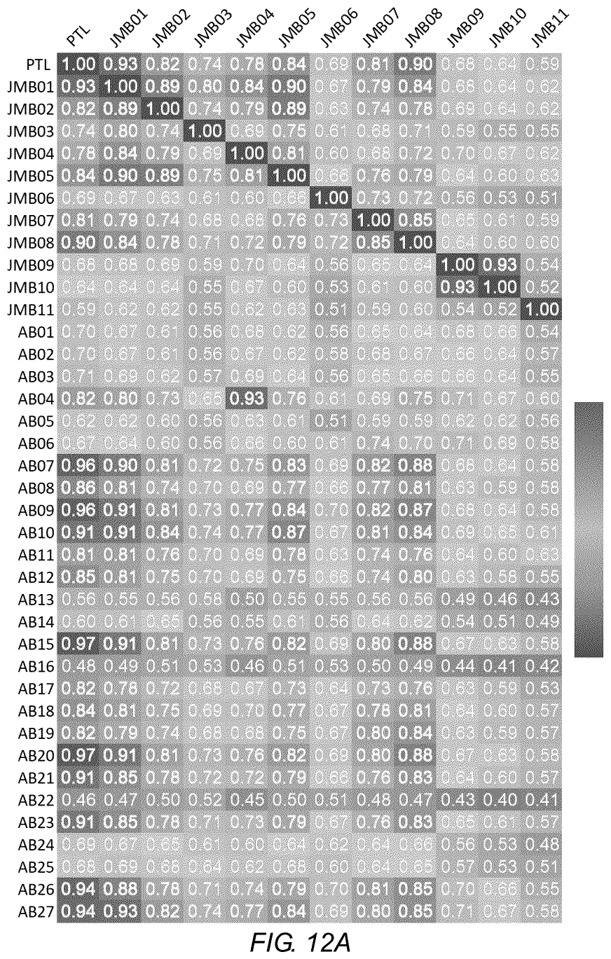

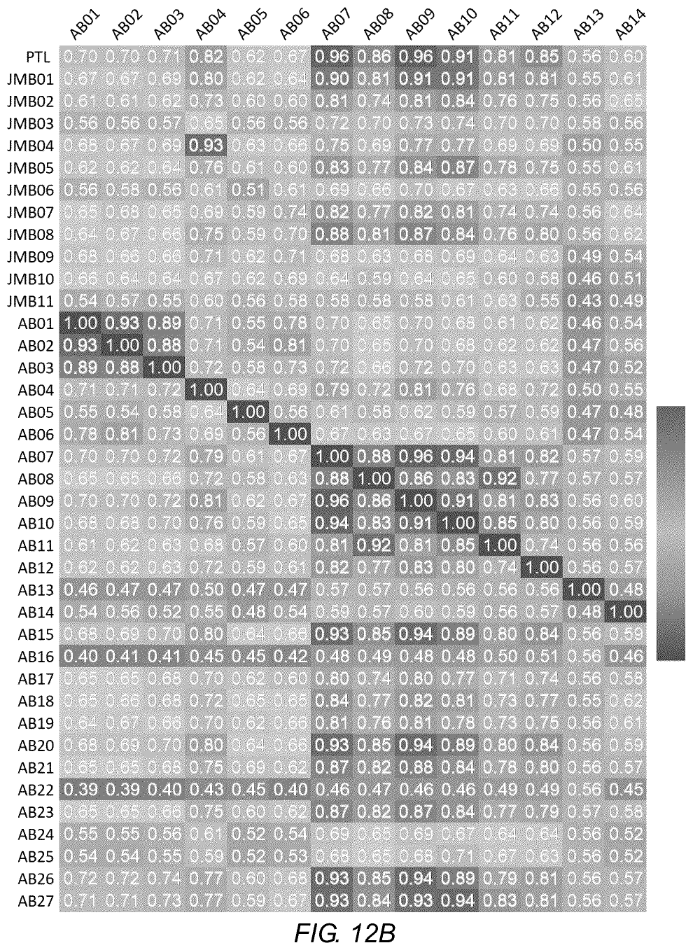

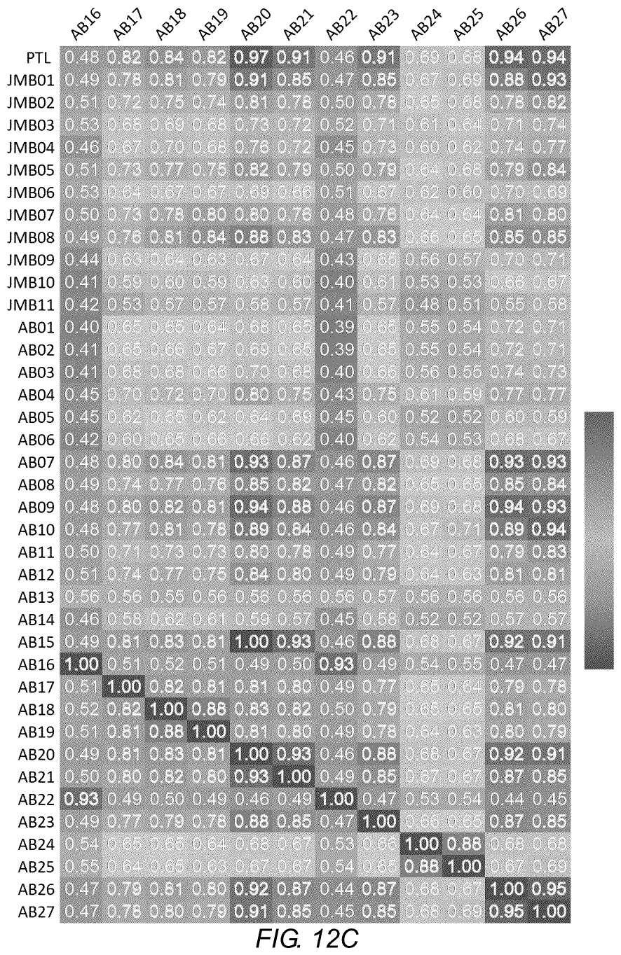

Chemoinformatic tools can be used to quantify the large amount of chemical and structural diversity encompassed by the compound collection in a more systematic manner The structural similarity between each compound vs PTL and any other member of the collection was measured using the Tanimoto coefficient, a measure of skeletal and structural similarity. As illustrated by the Tanimoto matrix (FIG. 12), this analysis indicated a significant degree of structural diversity across the collection, with most dissimilar compounds featuring Tanimoto coefficient as low as 0.45-0.50. As expected, compounds generated via similar synthetic strategies show greater degrees of similarity with one another vs. other members of the library (e.g., AB17-AB19 or AB01-AB03).

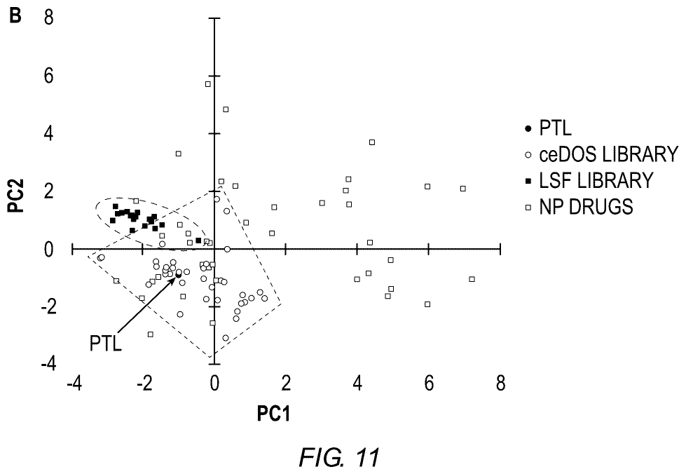

The chemical diversity encompassed by the compounds was also analyzed via principal component analysis (PCA) using a set of 13 different physicochemical descriptors, including H-bond donor/acceptor density, LogP, rotatable bonds, and ring/stereochemical complexity (FIG. 11). For comparison, the same analysis was conducted on (a) a diverse panel of 42 FDA-approved natural product-derived drugs, and (b) a panel of parthenolide analogs we previously generated via chemoenzymatic late-stage C—H functionalization (Kolev et al., ACS Chem Biol. (2014); 9(1):164-73). As shown in FIG. 11, this analysis showed that, similar to the NP drug library, this library encompasses a wide region of chemical space, which is particularly remarkable considering that the entire collection was derived from a single parent molecule, i.e. parthenolide (PTL). In addition, the library encompasses both a distinct and significantly larger area of the chemical space when compared to that covered by parthenolide (PTL) analogs obtained by ‘peripheral editing’ through late-stage C—H functionalization (Kolev et al., ACS Chem Biol. (2014); 9(1):164-73), which highlights the advantage of the present method toward generating new chemical diversity.

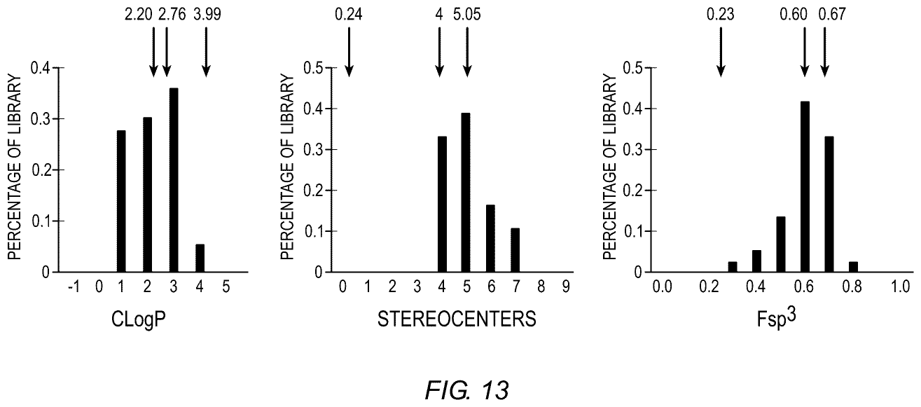

We further analyzed selected physiochemical properties of the compounds that are relevant for medicinal chemistry (e.g., n-octanol/water partition coefficient (ClogP), number of stereocenters, and fraction of sp3-hybridized carbons (Fsp3)). As shown in FIG. 13, 95% of the compounds possess a CLogP value between 1 and 3 (avg: 2.20), which falls within the optimal range (ClogP: 0-3) for good oral and intestinal absorption. High degrees of chirality and three-dimensionality (estimated by their Fsp³ values) are highly sought after in small-molecule libraries for drug discovery as these features are typically associated with higher target specificity and lower potential for off-target effects compared to achiral and ‘flat’ (sp²-rich) molecules. Reflecting its high degrees of three-dimensionality and stereochemical richness, the average number of stereocenters of the library is >20-fold greater than that of a traditional combinatorial chemistry library (5.05 vs. 0.24), whereas its average Fsp³ value is nearly threefold higher (0.60 vs. 0.23). Altogether, these data support the effectiveness of the present approach toward generating structurally diverse compounds that also possess desirable drug-like properties.

Experimental Procedures: Tanimoto similarity coefficients and physiochemical properties were calculated using the RDKit module rdkit.Chem. Molecules were imported as SMILES codes and fingerprint similarities were calculated using the default FingerprintSimilarity function. This process was repeated for all compounds and the resulting Tanimoto coefficients were arranged in a similarity matrix as shown in the manuscript and supporting information. The dataset for principal component analyses (PCA) was obtained using the software Instant JChem (ChemAxon) and the online Virtual Computational Chemistry Laboratory (VCCLAB, http://www.vcclab.org) calculators. Principal component analysis was performed using the prcomp function in the opensource software R. Principal components were then transferred into Excel to generate the graph shown in FIG. 11. The physiochemical properties on the graph are: H-bond donor density, H-bond acceptor density, rotatable bond density, number of rings, number of ring systems, largest ring size, fraction of sp3-hybridized atoms, relative polar surface area, distribution coefficient (logD), partition coefficient (logP), permeability surface area-product (logPS), stereochemical complexity, and ring complexity. The datapoints on the graph correspond to parthenolide (PTL), a representative selection of the natural product-like compounds described herein (CeDOS Library), a group of parthenolide analogs obtained via peripheral editing (LSF Library), and a group of FDA-approved natural product-based drugs (NP Drugs.

### REFERENCES

Each reference listed below is hereby incorporated by reference herein in its entirety.
Anderson, K. N. and B. E. Bejcek (2008). J Pharmacol Sci 106(2): 318-320.
Baeuerle, P. A. and T. Henkel (1994). Annual Review of Immunology 12: 141-179.
Barnes, P. J. and I. M. Adcock (1997). Trends in Pharmacological Sciences 18(2): 46-50.
Barnes, P. J. and M. Larin (1997). New England Journal of Medicine 336(15): 1066-1071.
Bessho, Y., D. R. Hodgson, et al. (2002). Nat Biotechnol 20(7): 723-728.
Bonnet, D. and J. E. Dick (1997). Nat Med 3(7): 730-737.
Castaneda-Acosta, J., N. H. Fischer, et al. (1993). J Nat Prod 56(1): 90-98.
Cheng, G. and L Xie (2011). Molecules 16(8): 6758-6768.
Cirino, P. C. and F. H. Arnold (2003). Angew. Chem. Int. Ed. Engl. 42(28): 3299-3301.
Costello, R. T., F. Mallet, et al. (2000). Cancer Research 60(16): 4403-4411.
Cox, C. V., R. S. Evely, et al. (2004). Blood 104(9): 2919-2925.
Cox, G. G., J. J. Kulagowski, et al. (1992). Synlett (12): 975-976.
Dedkova, L. M., N. E. Fahmi, et al. (2003). Journal of the American Chemical Society 125(22): 6616-6617.
Estabrook, N. C., H. Chin-Sinex, et al. (2011). Free Radic Biol Med 51(12): 2249-2258.
Galal, A. M., A. R. S. Ibrahim, et al. (1999). Phytochemistry 51(6): 761-765.
Garcia-Pineres, A. J., V. Castro, et al. (2001). Journal of Biological Chemistry 276(43): 39713-39720.
Garcia-Pineres, A. J., M. T. Lindenmeyer, et al. (2004). Life Sci 75(7): 841-856.
Ghantous, A., H. Gali-Muhtasib, et al. (2010). Drug Discov Today 15(15-16): 668-678.
Gopal, Y. N., T. S. Arora, et al. (2007). Chem Biol 14(7): 813-823.
Gopal, Y. N., E. Chanchorn, et al. (2009). Mol Cancer Ther 8(3): 552-562.
Graham, S. M., H. G. Jorgensen, et al. (2002). Blood 99(1): 319-325.
Guan, Y. H., B. Gerhard, et al. (2003). Blood 101(8): 3142-3149.
Guzman, M. L., L Karnischky, et al. (2004). Blood 104(11): 697a-697a.
Guzman, M. L., S. J. Neering, et al. (2001). Blood 98(8): 2301-2307.
Guzman, M. L., R. M. Rossi, et al. (2005). Blood 105(11): 4163-4169.
Guzman, M. L., R. M. Rossi, et al. (2006). Blood 108(11): 74a-74a.
Guzman, M. L., C. F. Swiderski, et al. (2002). Proc Nati Acad Sci USA 99(25): 16220-16225.
Han, C., F. J. Barrios, et al. (2009). J. Org. Chem. 74(18): 7176-7179.
Hehner, S. P., M. Heinrich, et al. (1998). J Biol Chem 273(3): 1288-1297.
Heptinstall, S., A. White, et al. (1985). Lancet 1(8437): 1071-1074.
Hirakawa, H. and T. Nagamune (2010). Chembiochem 11(11): 1517-1520.
Holcomb, B. K., M. T. Yip-Schneider, et al. (2012). J Gastrointest Surg 16(7): 1333-1340.
Holyoake, T., X. Y. Jiang, et al. (1999). Blood 94(6): 2056-2064.
Hwang, D., N. H. Fischer, et al. (1996). Biochem Biophys Res Commun 226(3): 810-818.
Hwang, D. R., C. W. Chang, et al. (2006). Bioorganic & Medicinal Chemistry 14(1): 83-91.
Idris, A. I., H. Libouban, et al. (2009). Mol Cancer Ther 8(8): 2339-2347.
Janecka, A., A. Wyrebska, et al. (2012). Drug Discov Today 17(11-12): 561-572.
Juliana, C., T. Fernandes-Alnemri, et al. (2010). J Biol Chem 285(13): 9792-9802.
Kawasaki, B. T., E. M. Hurt, et al. (2009). Prostate 69(8): 827-837.
Killmann, S. A. (1991). Baillieres Clin Haematol 4(3): 577-598.
Kim, I. H., S. W. Kim, et al. (2012). Exp Mol Med 44(7): 448-456.
Kim, J. H., L Liu, et al. (2005). Cancer Res 65(14): 6312-6320.
Knight, D. W. (1995). Nat Prod Rep 12(3): 271-276.
Kourouklis, D., H. Murakami, et al. (2005). Methods 36(3): 239-244.
Kreuger, M. R., S. Grootjans, et al. (2012). Anticancer Drugs 23(9): 883-896.
Kwok, B. H., B. Koh, et al. (2001). Chem Biol 8(8): 759-766.
Landwehr, M., M. Carbone, et al. (2007). Chem. Biol. 14(3): 269-278.
Lapidot, T., C. Sirard, et al. (1994). Nature 367(6464): 645-648.
Lesiak, K., K. Koprowska, et al. (2010). Melanoma Res 20(1): 21-34.
U, S. Y., L M. Podust, et al. (2007). J. Am. Chem. Soc. 129(43): 12940-12941.
U, Y., Y. Zhang, et al. (2012). Mol Med Rep 6(3): 477-482.
Uu, C. C. and P. G. Schultz (2010). Annu. Rev. Biochem. 79: 413-444.
Uu, Y., W. L Lu, et al. (2008). J Control Release 129(1): 18-25.
Uu, Z., S. Uu, et al. (2009). J Pharmacol Exp Ther 329(2): 505-514.
Merfort, I. (2011). Current Drug Targets 12(11): 1560-1573.
Murakami, H., A. Ohta, et al. (2006). Nat Methods 3(5): 357-359.
Nakshatri, H., S. E. Rice, et al. (2004). Oncogene 23(44): 7330-7344.
Nasim, S. and P. A. Crooks (2008). Bioorg Med Chem Lett 18(14): 3870-3873.
Neelakantan, S., S. Nasim, et al. (2009). Bioorg. Med. Chem. Lett. 19(15): 4346-4349.
Neukirch, H., N. C. Kaneider, et al. (2003). Bioorg Med Chem 11(7): 1503-1510.
Neumann, H., K. Wang, et al. (2010). Nature 464(7287): 441-444.
Oka, D., K. Nishimura, et al. (2007). International Journal of Cancer 120(12): 2576-2581.
Otey, C. R., M. Landwehr, et al. (2006). PLoS Biol. 4(5): e112.
Park, J. H., L Uu, et al. (2005). Cancer Res 65(7): 2804-2814.
Patel, N. M., S. Nozaki, et al. (2000). Oncogene 19(36): 4159-4169.
Peddibhotla, S., Y. J. Dang, et al. (2007). Journal of the American Chemical Society 129(40): 12222-12231.
Ralstin, M. C., E. A. Gage, et al. (2006). Mol Cancer Res 4(6): 387-399.
Rodriguez, E. A., H. A. Lester, et al. (2006). Proc Natl Acad Sci USA 103(23): 8650-8655.
Shanmugam, R., P. Kusumanchi, et al. (2011). Int J Cancer 128(10): 2481-2494.
Shanmugam, R., P. Kusumanchi, et al. (2010). Prostate 70(10): 1074-1086.
Siedle, B., A. J. Garcia-Pineres, et al. (2004). J Med Chem 47(24): 6042-6054.
Skalska, J., P. S. Brookes, et al. (2009). PLoS One 4(12): e8115.
Steele, A. J., D. T. Jones, et al. (2006). Leukemia 20(6): 1073-1079.
Sun, C. M., W. J. Syu, et al. (2003). J Nat Prod 66(9): 1175-1180.
Sun, Y., D. K. St Clair, et al. (2007). Mol Cancer Ther 6(9): 2477-2486.
Sun, Y., D. K. St Clair, et al. (2010). Cancer Res 70(7): 2880-2890.
Suvannasankha, A., C. D. Crean, et al. (2008). Clin Cancer Res 14(6): 1814-1822.
Sweeney, C. J., S. Mehrotra, et al. (2005). Mol Cancer Ther 4(6): 1004-1012.
Tiuman, T. S., T. Ueda-Nakamura, et al. (2005). Antimicrobial Agents and Chemotherapy 49(1): 176-182.
Tran, N. H., N. Huynh, et al. (2011). Chem Commun (Camb) 47(43): 11936-11938.
van der Donk, W. A. and H. Zhao (2003). Curr Opin Biotechnol 14(4): 421-426.
van Rhenen, A., N. Feller, et al. (2005). Clin Cancer Res 11(18): 6520-6527.
Wang, W., M. Adachi, et al. (2006). Apoptosis 11(12): 2225-2235.
Wang, W., M. Adachi, et al. (2009). Pancreas 38(4): e114-123.
Wen, J., K. R. You, et al. (2002). J Biol Chem 277(41): 38954-38964.
Won, Y. K., C. N. Ong, et al. (2005). Carcinogenesis 26(12): 2149-2156.
Won, Y. K., C. N. Ong, et al. (2004). Carcinogenesis 25(8): 1449-1458.
Woods, J. R., H. Mo, et al. (2011). J Med Chem 54(22): 7934-7941.
Wyrebska, A., K. Gach, et al. (2012). Chem Biol Drug Des 79(1): 112-120.
Yip-Schneider, M. T., H. Nakshatri, et al. (2005). Mol Cancer Ther 4(4): 587-594.
Yip-Schneider, M. T., H. Wu, et al. (2008). Pancreas 37(3): e45-53.
Zanotto-Filho, A., E. Braganhol, et al. (2011). Biochemical Pharmacology 81(3): 412-424.
Zhang, D., L. Qju, et al. (2009). Mol Cancer Res 7(7): 1139-1149.
Zhang, K., S. El Damaty, et al. (2011). J. Am. Chem. Soc. 133(10): 3242-3245.
Zhang, K., B. M. Shafer, et al. (2012). J Am Chem Soc 134(45): 18695-18704.
Zhang, S., C. N. Ong, et al. (2004). Cancer Lett 208(2): 143-153.
Zhang, S., C. N. Ong, et al. (2004). Cancer Lett 211(2): 175-188.
Zhao, H. and W. A. van der Donk (2003). Curr Opin Biotechnol 14(6): 583-589

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

While embodiments of the present disclosure have been particularly shown and described with reference to certain examples and features, it will be understood by one skilled in the art that various changes in detail may be made therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

1. A compound of the general formula selected from the group consisting of A1 through A45: 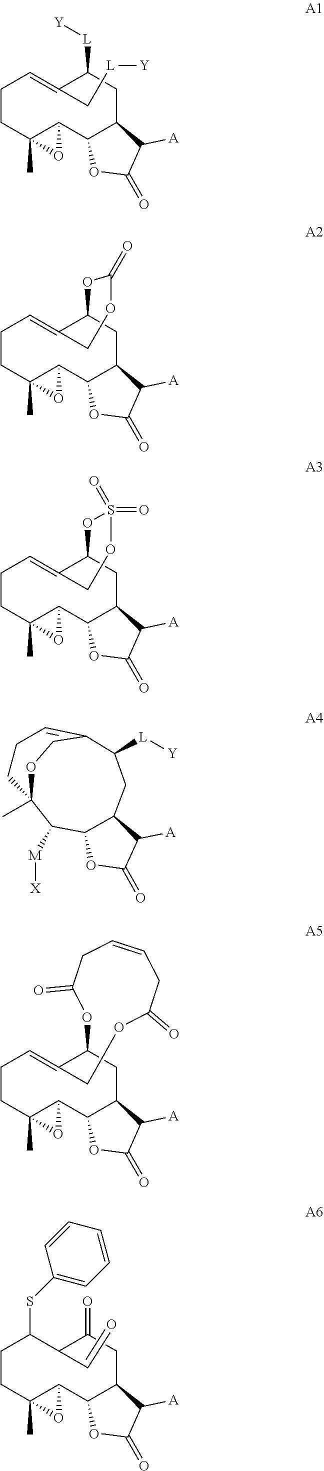 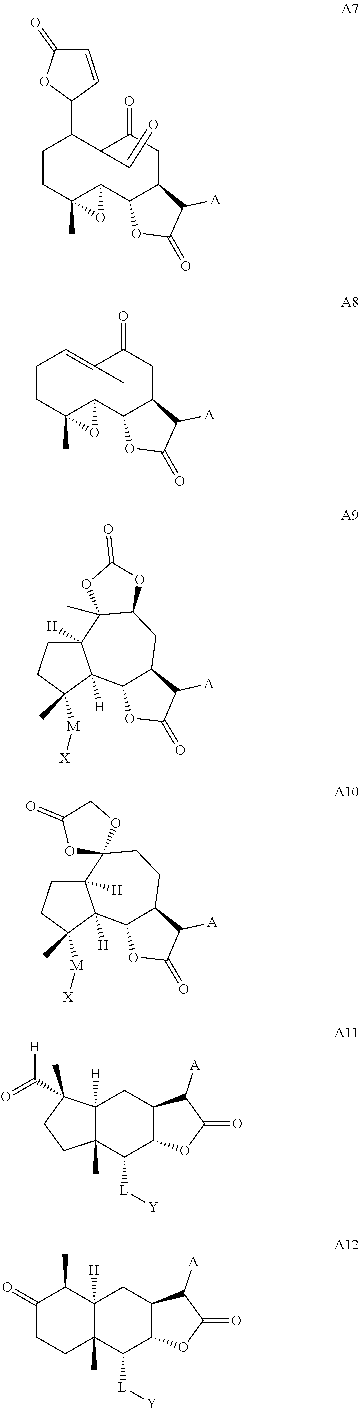 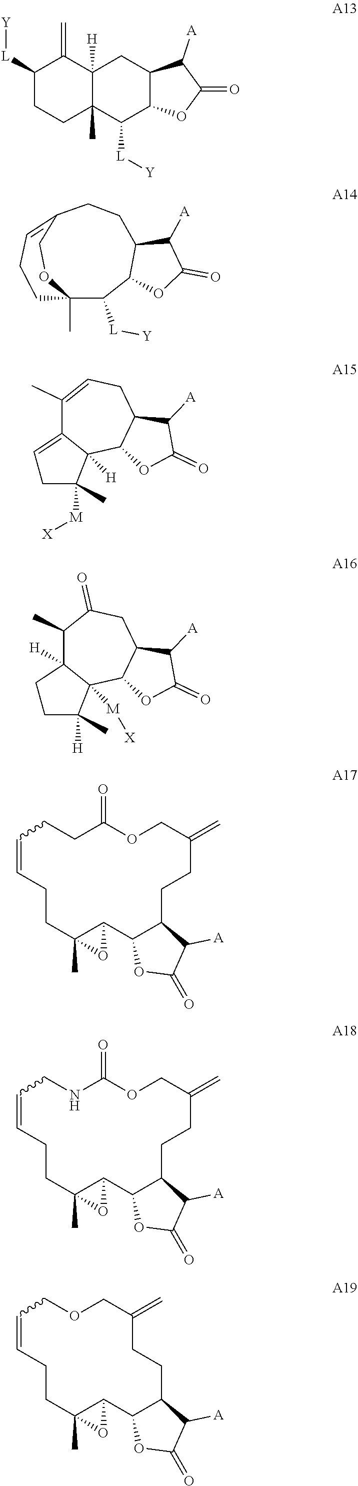 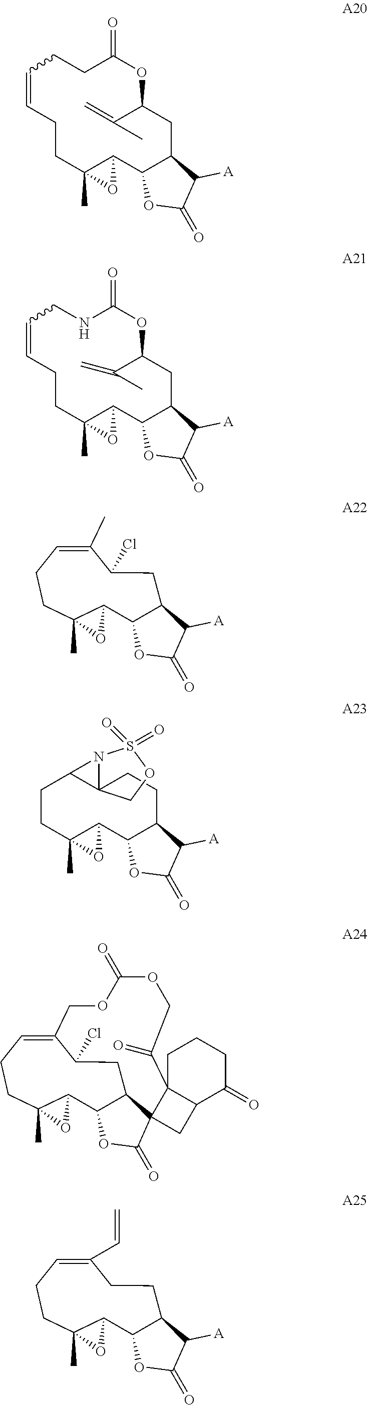 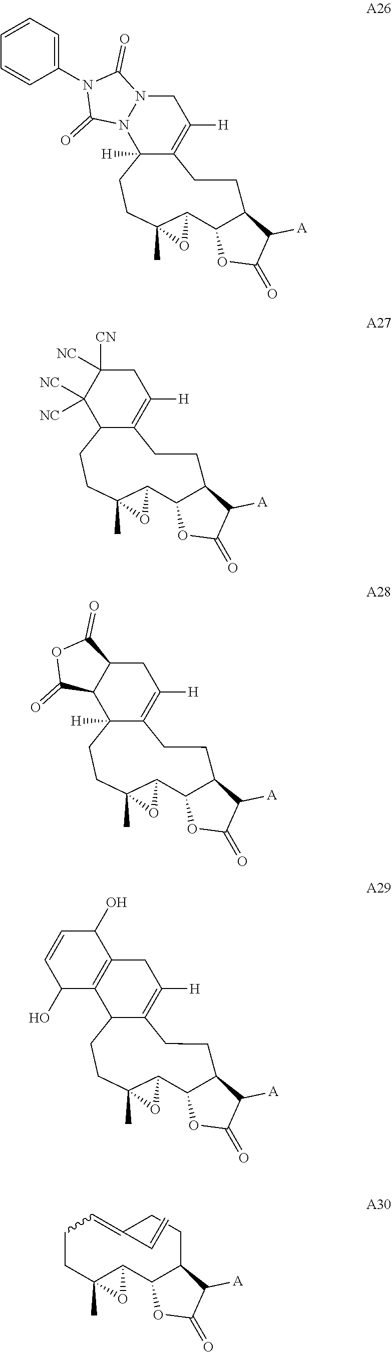 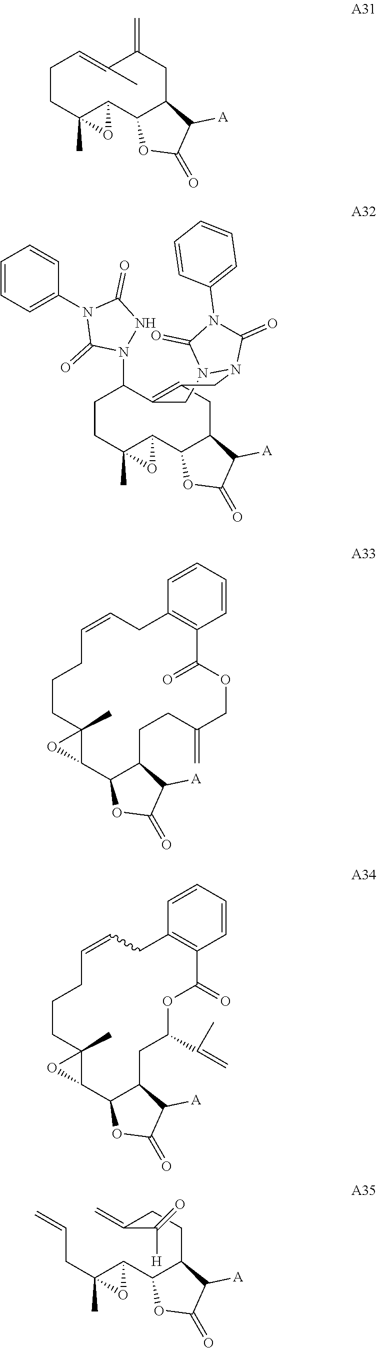 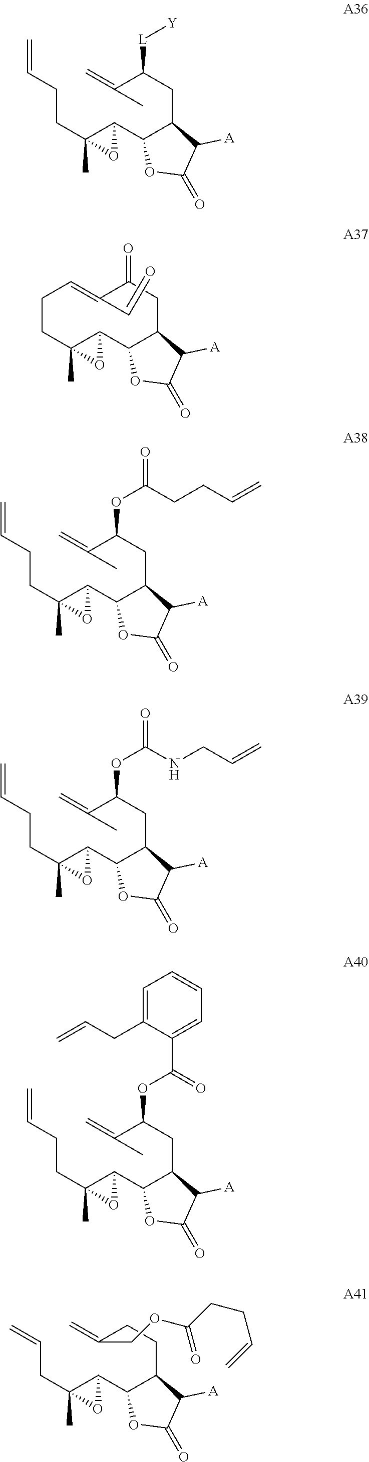 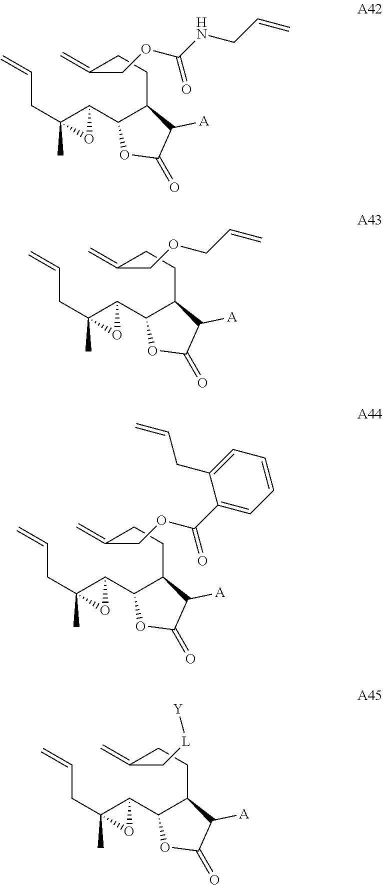
wherein
A is ═CH₂ or —CH₂R* wherein R* is an amino acid residue banded to the A methylene via a nitrogen or sulfur atom; or R* is —NR¹R², —NR¹C(O)R², —NR¹CO₂R², or —SR¹, wherein R¹ and R² are independently selected from the group consisting of H and an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group; or R* is —NR¹R², wherein R₁ and R₂ optionally together with the nitrogen atom form a an optionally substituted 5-12 membered ring, the ring optionally comprising at least one heteroatom or group selected from the group consisting of —CO—, —SO—, —SO₂—, and —PO—; L is —O—, —NH—, —NHC(O)—, —OC(O)—, —OC(O)NH—, —S—, —SO—, —SO₂—, —PO—, —OCH₂—, or a chemical bond connecting the carbon atom to Y;
Y represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group with the proviso that when -L-Y is hydrogen, -M-X is not hydroxyl, nor —C(O)CH₂CH₃ and A is not —CH₂—N(CH₃)₂, wherein -L-Y together with the carbon atom at position C2 optionally forms carbonyl; or
Y is absent and L represents a halogen atom, an azido group (—N₃), an optionally substituted triazole group, or a group —NR³R⁴, where R³ represents a hydrogen atom or an optionally substituted alkyl, alkenyl, or alkynyl group; R⁴ represents an optionally substituted alkyl, alkenyl, alkynyl, aryl, or heteroaryl group; or where R³ and R⁴ are connected together to form an optionally substituted heterocyclic group; and
M is —O— or —OC(O)— and X represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group.

2. The compound of claim 1, wherein L and M are independently —O— or —OC(O)—; Y and X are independently hydrogen, —(C₆-C₁₀) aryl, -(5-14 membered) heteroaryl, —(C₁-C₆) alkyl, —CH₂—(C₆-C₁₀) aryl, or —NH—(C₆-C₁₀) aryl; A is CH₂R*, where R* is selected from the group consisting of methylamino (—NH(CH₃)), dimethylamino (—N(CH₃)₂), methylethylamino (—N(CH₃)(CH₂CH₃)), methylpropylamino (—N(CH₃)(CH₂CH₂CH₃)), methylisopropylamino (—(CH₃)(CH₂(CH₃)₂), (—N(CH₃)(CH₂CH₂OH), pyrrolidine, piperidine, 4-methylpiperidine, 1-phenylmethanamine (—NCH₂Ph), and 2-phenylethanamine (—NCH₂CHPh).

3. The compound of claim 1, wherein A is ═CH₂, L and M are —O—; and X and Y are hydrogen.

4. The compound of claim 1, wherein the compound is selected from the group consisting of: 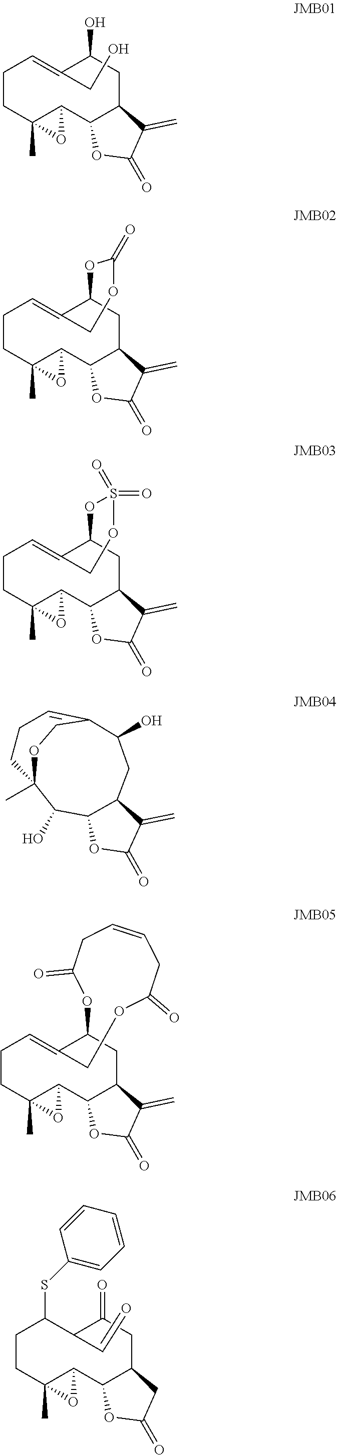 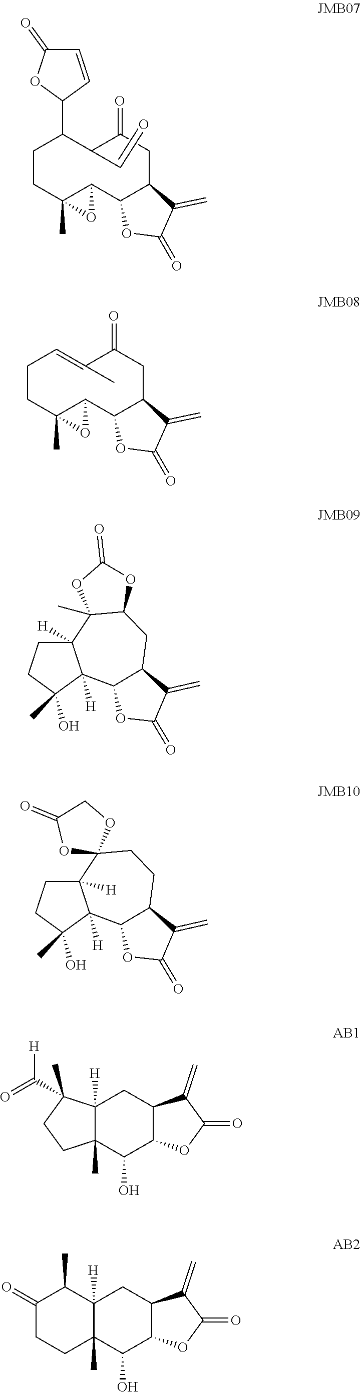 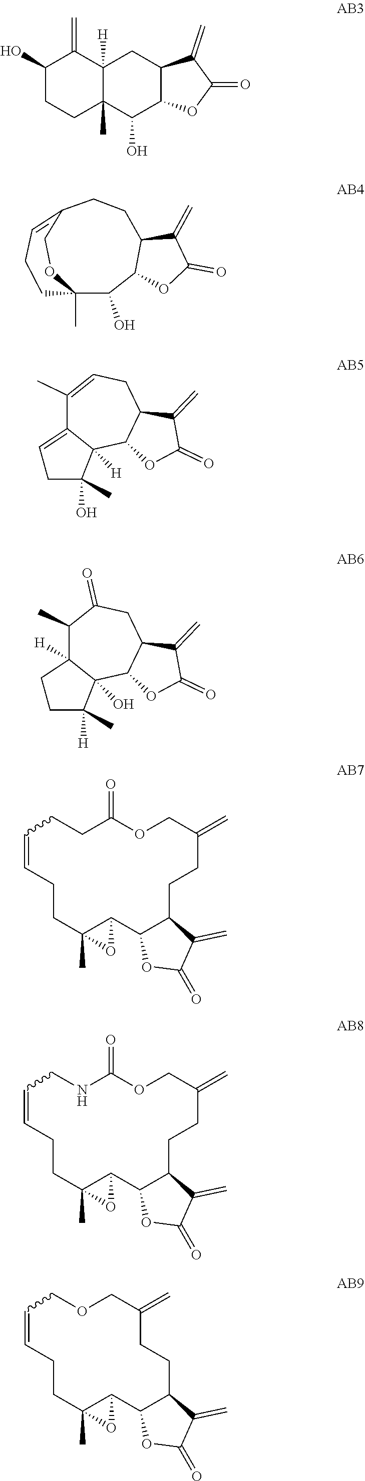 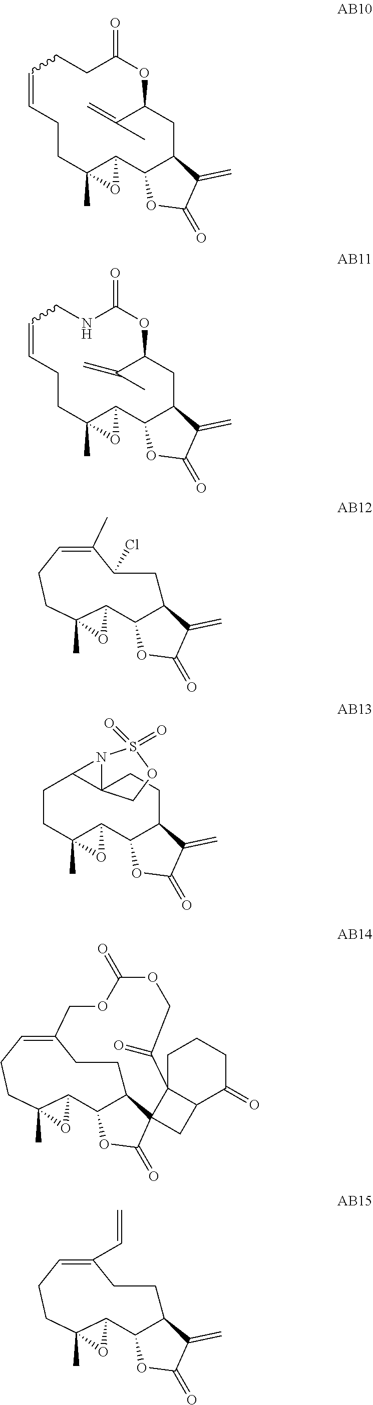 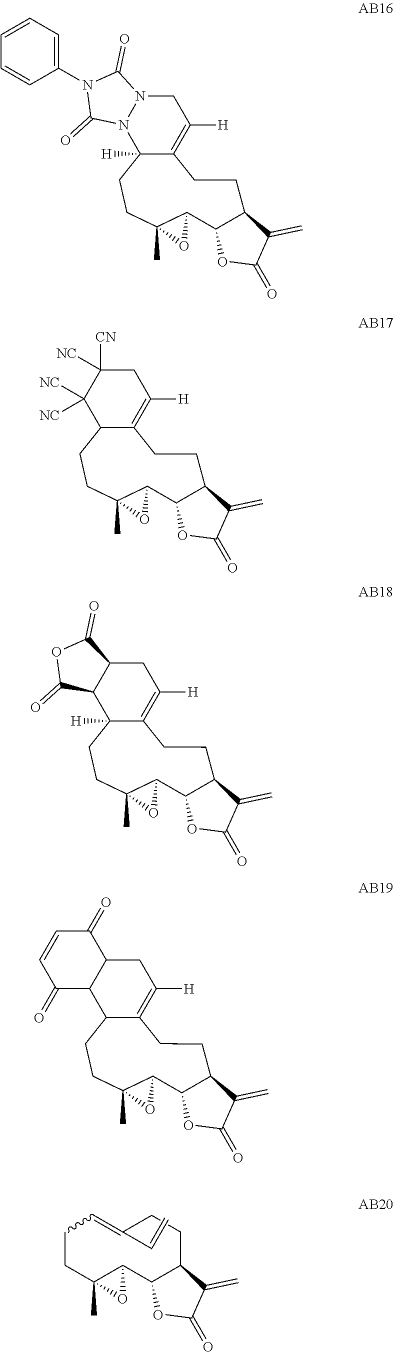 [SMILES: C=C1C(=O)O[C@@H]2[C@H]3O[C@]3(C)CC[C@@H]3C(=CCn4c(=O)n(-c5ccccc5)c(=O)n43)CC[C@@H]12 ; C=C1C(=O)O[C@@H]2[C@H]3O[C@]3(C)CCC3C(=CCC(C#N)(C#N)C3(C#N)C#N)CC[C@@H]12 ; C=C1C(=O)O[C@@H]2[C@H]3O[C@]3(C)CC[C@@H]3C(=CC[C@@H]4C(=O)OC(=O)[C@@H]43)CC[C@@H]12 ; C=C1C(=O)O[C@@H]2[C@H]3O[C@]3(C)CCC3C(=CCC4C(=O)C=CC(=O)C43)CC[C@@H]12 ; C=CC1=CCC[C@@]2(C)O[C@@H]2[C@H]2OC(=O)C(=C)[C@@H]2CC1] 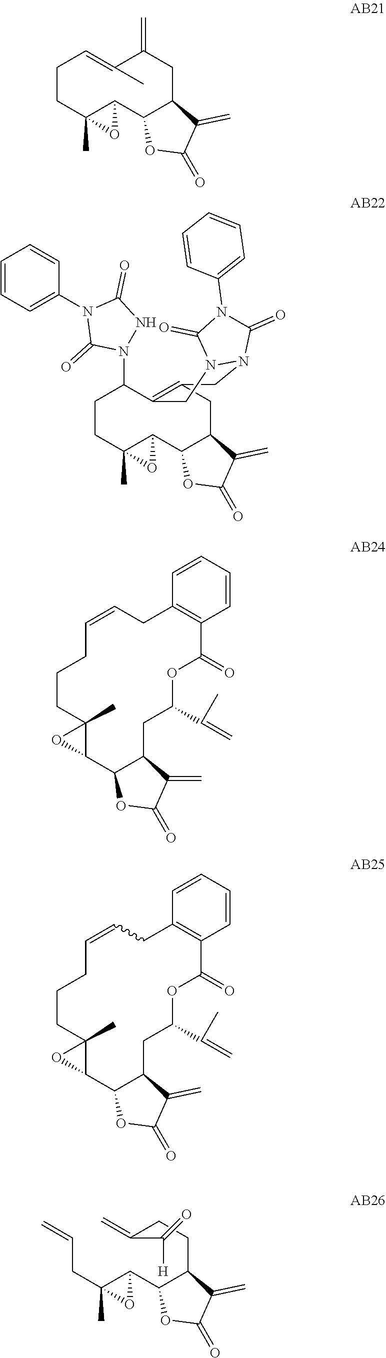 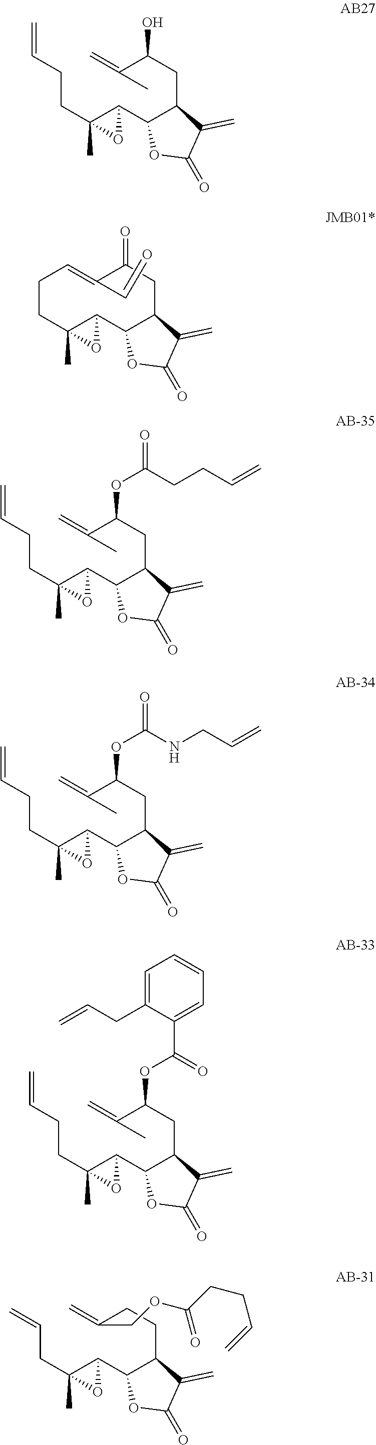 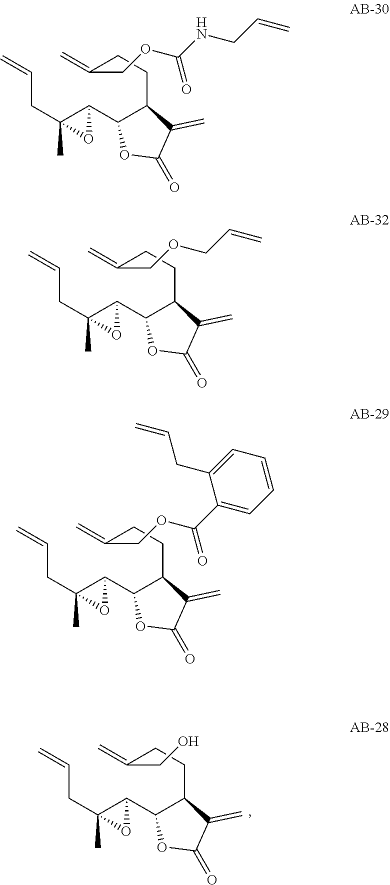
or a salt of any of the above compounds.

5. A pharmaceutical composition comprising a compound of claim 1 or a pharmaceutically acceptable salt thereof.

6. A pharmaceutical composition comprising a compound of claim 2 or a pharmaceutically acceptable salt thereof.

7. A pharmaceutical composition comprising the compound of claim 3 or a pharmaceutically acceptable salt thereof.

8. A pharmaceutical composition comprising a compound of claim 4 or a pharmaceutically acceptable salt thereof.

9. A pharmaceutical composition comprising a therapeutically effective amount of a compound of claim 1 or a pharmaceutically acceptable salt thereof.

10. A pharmaceutical composition comprising a therapeutically effective amount of a compound of claim 2 or a pharmaceutically acceptable salt.

11. A pharmaceutical composition comprising a therapeutically effective amount of the compound of claim 3 or a pharmaceutically acceptable salt thereof.

12. A pharmaceutical composition comprising a therapeutically effective amount of a compound of claim 4 or a pharmaceutically acceptable salt thereof.

13. (canceled)

14. A method for inhibiting cancer cell growth in a human, the method comprising the step of administering to the mammal afflicted with cancer an amount of a compound of claim 1 effective to inhibit the growth of the cancer cells.

15. The method of claim 14, wherein the cancer is acute myeloid leukemia (AML), chronic myeloid leukemia (CML), acute lymphoblastic leukemia (ALL), mantle cell lymphoma (MCL), or large B-cell lymphoma.

16. The method of claim 14, wherein the cancer is a prostate cancer, a brain cancer, a neuroblastoma, a lung cancer, a breast cancer, a skin cancer, a cervical cancer, a colon cancer, an ovary cancer, osteosarcoma or a pancreatic cancer.

17. The method of claim 14, wherein the compound is selected from the group consisting of:
AB24, AB25, AB10, JMB08, AB16, AB19, JMB03, AB20, AB26, AB08, AB24, JMB02 AB16, AB19, AB17, AB18, AB25, AB07, JMB06 and JMB07.

18. A method for treating an inflammatory condition in a human, the method comprising the step of administering to the human in need thereof, an amount of a compound of claim 1 effective to reduce, prevent, or control the condition.

19. (canceled)

20. (canceled)

21. A method for treating a patient with a cancerous tumor, comprising the step of administering to the patient an effective amount of a compound of claim 1.

22. (canceled)

23. The method of claim 21, wherein the tumor is acute myeloid leukemia (AML), chronic myeloid leukemia (CML), acute lymphoblastic leukemia (ALL), mantle cell lymphoma (MCL), or large B-cell lymphoma.

24. The method of claim 21, wherein the tumor is a prostate cancer, a brain cancer, a neuroblastoma, a lung cancer, a breast cancer, a skin cancer, a cervical cancer, a colon cancer, an ovary cancer, or a pancreatic cancer.

25. The method of claim 21, wherein the compound is selected from the group consisting of:
AB24, AB25, AB10, JMB08, AB16, AB19, JMB03, AB20, AB26, AB08, AB24, JMB02 AB16, AB19, AB17, AB18, AB25, AB07, JMB06 and JMB07.
